SEQUENCE LISTING (256 sequences)

SEQ ID NO: 1 - protein, 5 aa, Artificial Sequence
  Description of Artificial Sequence Synthetic peptide
DYYMS

SEQ ID NO: 2 - protein, 17 aa, Artificial Sequence
  Description of Artificial Sequence Synthetic peptide
YISSSGSTIYYADSVKG

SEQ ID NO: 3 - protein, 18 aa, Artificial Sequence
  Description of Artificial Sequence Synthetic peptide
IGRYSYGLRSYYYYGMDV

SEQ ID NO: 4 - protein, 30 aa, Artificial Sequence
  Description of Artificial Sequence Synthetic polypeptide
QVQLVESGGGLVKPGGSLRLSCAASGFTFS

SEQ ID NO: 5 - protein, 14 aa, Artificial Sequence
  Description of Artificial Sequence Synthetic peptide
WIRQAPGKGLEWVS

SEQ ID NO: 6 - protein, 32 aa, Artificial Sequence
  Description of Artificial Sequence Synthetic polypeptide
RFTISRDNAKNSLYLQMNSLRAEDTAVYYCAR

SEQ ID NO: 7 - protein, 11 aa, Artificial Sequence
  Description of Artificial Sequence Synthetic peptide
WGQGTTVTVSS

SEQ ID NO: 8 - protein, 127 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSS

SEQ ID NO: 9 - protein, 11 aa, Artificial Sequence
  Description of Artificial Sequence Synthetic peptide
RASQSISSYLN

SEQ ID NO: 10 - protein, 7 aa, Artificial Sequence
  Description of Artificial Sequence Synthetic peptide
AASSLQS

SEQ ID NO: 11 - protein, 9 aa, Artificial Sequence
  Description of Artificial Sequence Synthetic peptide
MQALQTPVT

SEQ ID NO: 12 - protein, 23 aa, Artificial Sequence
  Description of Artificial Sequence Synthetic peptide
DIQMTQSPSSLSASVGDRVTITC

SEQ ID NO: 13 - protein, 15 aa, Artificial Sequence
  Description of Artificial Sequence Synthetic peptide
WYQQKPGKAPKLLIY

SEQ ID NO: 14 - protein, 32 aa, Artificial Sequence
  Description of Artificial Sequence Synthetic polypeptide
GVPSRFSGSGSGTDFTLTISSLQPEDFGVYYC

SEQ ID NO: 15 - protein, 11 aa, Artificial Sequence
  Description of Artificial Sequence Synthetic peptide
FGQGTRLEIKR

SEQ ID NO: 16 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKR

SEQ ID NO: 17 - protein, 5 aa, Artificial Sequence
  Description of Artificial Sequence Synthetic peptide
DYYMS

SEQ ID NO: 18 - protein, 17 aa, Artificial Sequence
  Description of Artificial Sequence Synthetic peptide
YISSSGSTIYYADSVKG

SEQ ID NO: 19 - protein, 16 aa, Artificial Sequence
  Description of Artificial Sequence Synthetic peptide
VARQYRKSYYYYGMDV

SEQ ID NO: 20 - protein, 30 aa, Artificial Sequence; identical to SEQ ID NO: 4
  Description of Artificial Sequence Synthetic polypeptide

SEQ ID NO: 21 - protein, 14 aa, Artificial Sequence
  Description of Artificial Sequence Synthetic peptide
WIRQAPGKGLEWVS

SEQ ID NO: 22 - protein, 32 aa, Artificial Sequence
  Description of Artificial Sequence Synthetic polypeptide
RFTISRDNAKNSLYLQMNSLRAEDTAMYYCAR

SEQ ID NO: 23 - protein, 11 aa, Artificial Sequence
  Description of Artificial Sequence Synthetic peptide
WGQGTTVTVSS

SEQ ID NO: 24 - protein, 125 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAMYYCARVARQYRKSYYYYGMDVWGQGTTVTVSS

SEQ ID NO: 25 - protein, 11 aa, Artificial Sequence
  Description of Artificial Sequence Synthetic peptide
RASQSISSYLN

SEQ ID NO: 26 - protein, 7 aa, Artificial Sequence
  Description of Artificial Sequence Synthetic peptide
AASSLQS

SEQ ID NO: 27 - protein, 9 aa, Artificial Sequence
  Description of Artificial Sequence Synthetic peptide
MQALQTPLT

SEQ ID NO: 28 - protein, 23 aa, Artificial Sequence
  Description of Artificial Sequence Synthetic peptide
DIQMTQSPSSLSASVGDRVTITC

SEQ ID NO: 29 - protein, 15 aa, Artificial Sequence
  Description of Artificial Sequence Synthetic peptide
WYQQKPGKAPKLLIY

SEQ ID NO: 30 - protein, 32 aa, Artificial Sequence
  Description of Artificial Sequence Synthetic polypeptide
GVPSRFSGSGSGTDFTLTISSLQPEDVGVYYC

SEQ ID NO: 31 - protein, 11 aa, Artificial Sequence
  Description of Artificial Sequence Synthetic peptide
FGGGTKLEIKR

SEQ ID NO: 32 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDVGVYYCMQALQTPLTFGGGTKLEIKR

SEQ ID NO: 33 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 381 bp, Artificial Sequence
  Description of Artificial Sequence Synthetic polynucleotide
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctca

SEQ ID NO: 36 - DNA, 324 bp, Artificial Sequence
  Description of Artificial Sequence Synthetic polynucleotide
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgt

SEQ ID NO: 37 - DNA, 375 bp, Artificial Sequence
  Description of Artificial Sequence Synthetic polynucleotide
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggccatgtattactgtgcgagagtggcacggcaatatcggaaaagctactactactacggtatggacgtctggggccaagggaccacggtcaccgtctcctca

SEQ ID NO: 38 - DNA, 324 bp, Artificial Sequence
  Description of Artificial Sequence Synthetic polynucleotide
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggatgttggggtttattactgcatgcaagctctacaaactccgctcactttcggcggagggaccaagctggagatcaaacgt

SEQ ID NO: 39 - protein, 15 aa, Homo sapiens
EPKSCDKTHTCPPCP

SEQ ID NO: 40 - DNA, 45 bp, Homo sapiens
gagcccaaatcttgtgacaaaactcacacatgcccaccgtgccca

SEQ ID NO: 41 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 651 bp, Homo sapiens
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - protein, 87 aa, Artificial Sequence
  Description of Artificial Sequence Synthetic polypeptide
  NON_CONS (30)..(31): Residues at these positions can be separated by a CDR1 sequence of Variable Heavy Region of unknown length
  NON_CONS (44)..(45): Residues at these positions can be separated by a CDR2 sequence of Variable Heavy Region of unknown length
  NON_CONS (76)..(77): Residues at these positions can be separated by a CDR3 sequence of Variable Heavy Region of unknown length
QVQLVESGGGLVKPGGSLRLSCAASGFTFSWIRQAPGKGLEWVSRFTISRDNAKNSLYLQMNSLRAEDTAVYYCARWGQGTTVTVSS

SEQ ID NO: 44 - protein, 81 aa, Artificial Sequence
  Description of Artificial Sequence Synthetic polypeptide
  NON_CONS (23)..(24): Residues at these positions can be separated by a CDR1 sequence of Variable Light Region of unknown length
  NON_CONS (38)..(39): Residues at these positions can be separated by a CDR2 sequence of Variable Light Region of unknown length
  NON_CONS (70)..(71): Residues at these positions can be separated by a CDR3 sequence of Variable Light Region of unknown length
DIQMTQSPSSLSASVGDRVTITCWYQQKPGKAPKLLIYGVPSRFSGSGSGTDFTLTISSLQPEDFGVYYCFGQGTRLEIKR

SEQ ID NO: 45 - protein, 260 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKRGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSS

SEQ ID NO: 46 - DNA, 780 bp, Artificial Sequence
  Description of Artificial Sequence Synthetic polynucleotide
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgtggaggcggtgggagcgggggcggaggtagtggcggtggagggagcggtggcgggggaagtggagggggtggcagccaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctca

SEQ ID NO: 47 - protein, 250 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSS

SEQ ID NO: 48 - DNA, 750 bp, Artificial Sequence
  Description of Artificial Sequence Synthetic polynucleotide
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgtggcggtggagggagcggtggcgggggaagtggagggggtggcagccaagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctca

SEQ ID NO: 49 - protein, 260 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSSGGGGSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKR

SEQ ID NO: 50 - DNA, 780 bp, Artificial Sequence
  Description of Artificial Sequence Synthetic polynucleotide
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctcaggaggcggtgggagcgggggcggaggtagtggcggtggagggagcggtggcgggggaagtggagggggtggcagcgatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgt

SEQ ID NO: 51 - protein, 250 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKR

SEQ ID NO: 52 - DNA, 750 bp, Artificial Sequence
  Description of Artificial Sequence Synthetic polynucleotide
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcacggattgggcggtacagctatggtctccggtcctactactactacggtatggacgtctggggccaagggaccacggtcaccgtctcctcaggcggtggagggagcggtggcgggggaagtggagggggtggcagcgatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggattttggggtttattactgcatgcaagctctacaaactccggtcaccttcggccaagggacacgactggagattaaacgt

SEQ ID NO: 53 - protein, 87 aa, Artificial Sequence
  Description of Artificial Sequence Synthetic polypeptide
  NON_CONS (30)..(31): Residues at these positions can be separated by a CDR1 sequence of Variable Heavy Region of unknown length
  NON_CONS (44)..(45): Residues at these positions can be separated by a CDR2 sequence of Variable Heavy Region of unknown length
  NON_CONS (76)..(77): Residues at these positions can be separated by a CDR3 sequence of Variable Heavy Region of unknown length
QVQLVESGGGLVKPGGSLRLSCAASGFTFSWIRQAPGKGLEWVSRFTISRDNAKNSLYLQMNSLRAEDTAMYYCARWGQGTTVTVSS

SEQ ID NO: 54 - protein, 81 aa, Artificial Sequence
  Description of Artificial Sequence Synthetic polypeptide
  NON_CONS (23)..(24): Residues at these positions can be separated by a CDR1 sequence of Variable Light Region of unknown length
  NON_CONS (38)..(39): Residues at these positions can be separated by a CDR2 sequence of Variable Light Region of unknown length
  NON_CONS (70)..(71): Residues at these positions can be separated by a CDR3 sequence of Variable Light Region of unknown length
DIQMTQSPSSLSASVGDRVTITCWYQQKPGKAPKLLIYGVPSRFSGSGSGTDFTLTISSLQPEDVGVYYCFGGGTKLEIKR

SEQ ID NO: 55 - protein, 248 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKRGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAMYYCARVARQYRKSYYYYGMDVWGQGTTVTVSS

SEQ ID NO: 56 - protein, 248 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAMYYCARVARQYRKSYYYYGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKR

SEQ ID NO: 57 - protein, 91 aa, Artificial Sequence
  Description of Artificial Sequence Synthetic polypeptide
  NON_CONS (25)..(26): Residues at these positions can be separated by a CDR1 sequence of Variable Heavy Region of unknown length
  NON_CONS (42)..(43): Residues at these positions can be separated by a CDR2 sequence of Variable Heavy Region of unknown length
  NON_CONS (80)..(81): Residues at these positions can be separated by a CDR3 sequence of Variable Heavy Region of unknown length
EVQLVQSGGGLVQPGGSLRLSCSASMHWVRQAPGKGLEYVSAYYADSVKGRFTISRDNSKNTLYLQMNSLRAEDTAVYYCWGQGTMVTVSS

SEQ ID NO: 58 - protein, 25 aa, Artificial Sequence
  Description of Artificial Sequence Synthetic peptide
EVQLVQSGGGLVQPGGSLRLSCSAS

SEQ ID NO: 59 - protein, 17 aa, Artificial Sequence
  Description of Artificial Sequence Synthetic peptide
MHWVRQAPGKGLEYVSA

SEQ ID NO: 60 - protein, 38 aa, Artificial Sequence
  Description of Artificial Sequence Synthetic polypeptide
YYADSVKGRFTISRDNSKNTLYLQMNSLRAEDTAVYYC

SEQ ID NO: 61 - protein, 11 aa, Artificial Sequence
  Description of Artificial Sequence Synthetic peptide
WGQGTMVTVSS

SEQ ID NO: 62 - protein, 90 aa, Artificial Sequence
  Description of Artificial Sequence Synthetic polypeptide
  NON_CONS (26)..(27): Residues at these positions can be separated by a CDR1 sequence of Variable Light Region of unknown length
  NON_CONS (43)..(44): Residues at these positions can be separated by a CDR2 sequence of Variable Light Region of unknown length
  NON_CONS (79)..(80): Residues at these positions can be separated by a CDR3 sequence of Variable Light Region of unknown length
EATLTQSPSSLSASVGDRVTITCRASLNWYQQKPGRAPKLLIYSLQSGVPSRFSGSGSGTDFTLTISSLQPEDFATYYCFGQGTRLEIKR

SEQ ID NO: 63 - protein, 26 aa, Artificial Sequence
  Description of Artificial Sequence Synthetic peptide
EATLTQSPSSLSASVGDRVTITCRAS

SEQ ID NO: 64 - protein, 17 aa, Artificial Sequence
  Description of Artificial Sequence Synthetic peptide
LNWYQQKPGRAPKLLIY

SEQ ID NO: 65 - protein, 36 aa, Artificial Sequence
  Description of Artificial Sequence Synthetic polypeptide
SLQSGVPSRFSGSGSGTDFTLTISSLQPEDFATYYC

SEQ ID NO: 66 - protein, 91 aa, Artificial Sequence
  Description of Artificial Sequence Synthetic polypeptide
  NON_CONS (25)..(26): Residues at these positions can be separated by a CDR1 sequence of Variable Heavy Region of unknown length
  NON_CONS (42)..(43): Residues at these positions can be separated by a CDR2 sequence of Variable Heavy Region of unknown length
  NON_CONS (80)..(81): Residues at these positions can be separated by a CDR3 sequence of Variable Heavy Region of unknown length
EVQLVQSGGGLVQPGGSLRLSCAASMTWVRQAPGKGLEWVSTYYADSVKGRFTISRDNDKNTLYLQMNSLRADDTAVYYCWGQGTLVTVSS

SEQ ID NO: 67 - protein, 25 aa, Artificial Sequence
  Description of Artificial Sequence Synthetic peptide
EVQLVQSGGGLVQPGGSLRLSCAAS

SEQ ID NO: 68 - protein, 17 aa, Artificial Sequence
  Description of Artificial Sequence Synthetic peptide
MTWVRQAPGKGLEWVST

SEQ ID NO: 69 - protein, 38 aa, Artificial Sequence
  Description of Artificial Sequence Synthetic polypeptide
YYADSVKGRFTISRDNDKNTLYLQMNSLRADDTAVYYC

SEQ ID NO: 70 - protein, 11 aa, Artificial Sequence
  Description of Artificial Sequence Synthetic peptide
WGQGTLVTVSS

SEQ ID NO: 71 - protein, 89 aa, Artificial Sequence
  Description of Artificial Sequence Synthetic polypeptide
  NON_CONS (25)..(26): Residues at these positions can be separated by a CDR1 sequence of Variable Light Region of unknown length
  NON_CONS (42)..(43): Residues at these positions can be separated by a CDR2 sequence of Variable Light Region of unknown length
  NON_CONS (78)..(79): Residues at these positions can be separated by a CDR3 sequence of Variable Light Region of unknown length
QSVVTQPPSASGSPGQSVTISCTGTVSWYQQHPGKAPKLLIYERPSGVPDRFSGSKSDNKASLTIIGLQTEDEADYYCFGTGTKLTVLR

SEQ ID NO: 72 - protein, 25 aa, Artificial Sequence
  Description of Artificial Sequence Synthetic peptide
QSVVTQPPSASGSPGQSVTISCTGT

SEQ ID NO: 73 - protein, 17 aa, Artificial Sequence
  Description of Artificial Sequence Synthetic peptide
VSWYQQHPGKAPKLLIY

SEQ ID NO: 74 - protein, 36 aa, Artificial Sequence
  Description of Artificial Sequence Synthetic polypeptide
ERPSGVPDRFSGSKSDNKASLTIIGLQTEDEADYYC

SEQ ID NO: 75 - protein, 11 aa, Artificial Sequence
  Description of Artificial Sequence Synthetic peptide
FGTGTKLTVLR

SEQ ID NO: 76 - protein, 15 aa, Artificial Sequence
  Description of Artificial Sequence Synthetic peptide
GGGGSGGSGSGGGGS

SEQ ID NO: 77 - protein, 15 aa, Artificial Sequence
  Description of Artificial Sequence Synthetic peptide
GGGGSGGSGSSGGGS

SEQ ID NO: 78 - protein, 15 aa, Artificial Sequence
  Description of Artificial Sequence Synthetic peptide
GGGGSGGGGSGGGGS

SEQ ID NO: 79 - protein, 15 aa, Artificial Sequence
  Description of Artificial Sequence Synthetic peptide
GGGGSGSGASSGGGS

SEQ ID NO: 80 - protein, 15 aa, Artificial Sequence
  Description of Artificial Sequence Synthetic peptide
GGGGSGSGASGGGGS

SEQ ID NO: 81 - protein, 15 aa, Artificial Sequence
  Description of Artificial Sequence Synthetic peptide
SGGGSGGGGSGGGGS

SEQ ID NO: 82 - protein, 18 aa, Artificial Sequence
  Description of Artificial Sequence Synthetic peptide
GSTSGSGKPGSGEGSTKG

SEQ ID NO: 83 - protein, 19 aa, Artificial Sequence
  Description of Artificial Sequence Synthetic peptide
PNGASQSSSASHTGSAPGS

SEQ ID NO: 84 - protein, 19 aa, Artificial Sequence
  Description of Artificial Sequence Synthetic peptide
PNGASNSGSAPDTSSAPGS

SEQ ID NO: 85 - protein, 19 aa, Artificial Sequence
  Description of Artificial Sequence Synthetic peptide
PNGASHSGSAPNTSSAPGS

SEQ ID NO: 86 - protein, 19 aa, Artificial Sequence
  Description of Artificial Sequence Synthetic peptide
PNGASESGSASKTSSASGS

SEQ ID NO: 87 - protein, 19 aa, Artificial Sequence
  Description of Artificial Sequence Synthetic peptide
PNGASNSGSAPKTGSASGS

SEQ ID NO: 88 - protein, 19 aa, Artificial Sequence
  Description of Artificial Sequence Synthetic peptide
PNGASKSGSASQTSSAPGS

SEQ ID NO: 89 - protein, 19 aa, Artificial Sequence
  Description of Artificial Sequence Synthetic peptide
PNGASHSSSASQTGSAPGS

SEQ ID NO: 90 - protein, 12 aa, Artificial Sequence
  Description of Artificial Sequence Synthetic peptide
PNGASKRSAPGS

SEQ ID NO: 91 - protein, 19 aa, Artificial Sequence
  Description of Artificial Sequence Synthetic peptide
PNGASHSGSAPHTSSASGS

SEQ ID NO: 92 - protein, 15 aa, Artificial Sequence
  Description of Artificial Sequence Synthetic peptide
RGRGRGRGRSRGGGS

SEQ ID NO: 93 - protein, 15 aa, Artificial Sequence
  Description of Artificial Sequence Synthetic peptide
SHGGSHGGGSGGGGS

SEQ ID NO: 94 - protein, 5 aa, Artificial Sequence
  Description of Artificial Sequence Synthetic peptide
GQAGR

SEQ ID NO: 95 - protein, 8 aa, Artificial Sequence
  Description of Artificial Sequence Synthetic peptide
GGGSGGGG

SEQ ID NO: 96 - protein, 5 aa, Artificial Sequence
  Description of Artificial Sequence Synthetic peptide
SGGGG

SEQ ID NO: 97 - protein, 5 aa, Artificial Sequence
  Description of Artificial Sequence Synthetic peptide
GGGGS

SEQ ID NO: 98 - protein, 30 aa, Artificial Sequence
  Description of Artificial Sequence Synthetic polypeptide
GGGGSGGGGSGGGGSGGGGSGGGGSGGGGS

SEQ ID NO: 99 - protein, 16 aa, Artificial Sequence
  Description of Artificial Sequence Synthetic peptide
GGGGGSGGGGSGGGGS

SEQ ID NO: 100 - protein, 26 aa, Artificial Sequence
  Description of Artificial Sequence Synthetic peptide
GGGGGSGGGGSGGGGSGGGGSGGGGS

SEQ ID NO: 101 - protein, 25 aa, Artificial Sequence
  Description of Artificial Sequence Synthetic peptide
GGGGSGGGGSGGGGSGGGGSGGGGS

SEQ ID NO: 102 - protein, 3 aa, Artificial Sequence
  Description of Artificial Sequence Synthetic peptide
AAA

SEQ ID NO: 103 - protein, 22 aa, Homo sapiens
MDMRVPAQLLGLLLLWLRGARC

SEQ ID NO: 104 - DNA, 66 bp, Homo sapiens
atggatatgagggtccccgcacaattgctcggtctccttctgctttggctccggggcgcgcggtgc

SEQ ID NO: 105 - protein, 15 aa, Homo sapiens
EPKSCDKTHTCPPCP

SEQ ID NO: 106 - protein, 15 aa, Homo sapiens
DKTHTCPPCPAPPVA

SEQ ID NO: 107 - protein, 12 aa, Homo sapiens
ESKYGPPCPPCP

SEQ ID NO: 108 - protein, 12 aa, Homo sapiens
ESKYGPPCPSCP

SEQ ID NO: 109 - protein, 49 aa, Homo sapiens
KPTTTPAPRPPTPAPTIASQPLSLRPEACRPAAGGAVHTRGLDFACDIY

SEQ ID NO: 110 - protein, 39 aa, Homo sapiens
IEVMYPPPYLDNERSNGTIIHVKGKHLCPSPLFPGPSKP

SEQ ID NO: 111 - protein, 47 aa, Homo sapiens
TTTPAPRPPTPAPTIASQPLSLRPEACRPAAGGAVHTRGLDFACDIY

SEQ ID NO: 112 - protein, 45 aa, Homo sapiens
TTTPAPRPPTPAPTIASQPLSLRPEACRPAAGGAVHTRGLDFACD

SEQ ID NO: 113 - protein, 21 aa, Homo sapiens
LCYLLDGILFIYGVILTALFL

SEQ ID NO: 114 - protein, 22 aa, Homo sapiens
MALIVLGGVAGLLLFIGLGIFF

SEQ ID NO: 115 - protein, 21 aa, Homo sapiens
IYIWAPLAGTCGVLLLSLVIT

SEQ ID NO: 116 - protein, 24 aa, Homo sapiens
IYIWAPLAGTCGVLLLSLVITLYC

SEQ ID NO: 117 - protein, 22 aa, Homo sapiens
IWAPLAGTCGVLLLSLVITLYC

SEQ ID NO: 118 - protein, 19 aa, Homo sapiens
IWAPLAGTCGVLLLSLVIT

SEQ ID NO: 119 - protein, 27 aa, Homo sapiens
FWVLVVVGGVLACYSLLVTVAFIIFWV

SEQ ID NO: 120 - protein, 21 aa, Homo sapiens
FWLPIGCAAFVVVCILGCILI

SEQ ID NO: 121 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 122 - protein, 42 aa, Homo sapiens
KRGRKKLLYIFKQPFMRPVQTTQEEDGCSCRFPEEEEGGCEL

SEQ ID NO: 123 - protein, 37 aa, Homo sapiens
KRGRKKLLYIFKQPFMRPVQTTQEEDGCSCRFPEEEE

SEQ ID NO: 124 - protein, 41 aa, Homo sapiens
RSKRSRLLHSDYMNMTPRRPGPTRKHYQPYAPPRDFAAYRS

SEQ ID NO: 125 - protein, 42 aa, Homo sapiens
ALYLLRRDQRLPPDAHKPPGGGSFRTPIQEEQADAHSTLAKI

SEQ ID NO: 126 - protein, 38 aa, Homo sapiens
CWLTKKKYSSSVHDPNGEYMFMRAVNTAKKSRLTDVTL

SEQ ID NO: 127 - protein, 24 aa, Homo sapiens
LCARPRRSPAQEDGKVYINMPGRG

SEQ ID NO: 128 - protein, 52 aa, Homo sapiens
YFLGRLVPRGRGAAEAATRKQRITETESPYQELQGQRSDVYSDLNTQRPYYK

SEQ ID NO: 129 - protein, 48 aa, Homo sapiens
QRRKYRSNKGESPVEPAEPCHYSCPREEEGSTIPIQEDYRKPEPACSP

SEQ ID NO: 130 - protein, 520 aa, Artificial Sequence
  Description of Artificial Sequence Synthetic polypeptide
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKGGGGGSGGGGSGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 131 - DNA, 1560 bp, Artificial Sequence
  Description of Artificial Sequence Synthetic polynucleotide
atggatatgagggtccccgcacaattgctcggtctccttctgctttggctccggggcgcgcggtgcgatattcagatgacccagagcccgtcctcactttcagctagcgtcggtgaccgggtgaccattacatgcagggcaagtcaaagtatcagctcatatttgaattggtaccagcagaaaccaggtaaagcccccaagctccttatttacgcagcttcatcactgcaatccggagtaccgtctagattctctggcagtggcagcgggaccgactttacgttgactatttcttcccttcaacctgaggactttggtgtttactactgcatgcaagcgctccagactcctgttacatttggccagggcacccggcttgaaattaagggcgggggcggtggaagtgggggaggaggatcagggggtggtgggtctggagggggaggtagtggcggtggaggaagtcaagtgcaattggtagagagcggcggcggactagtaaaacctgggggaagcctgcggctgtcctgcgcagcaagcggattcactttcagtgattattacatgagctggattcgccaggcgcctggtaaaggtttggagtgggtgtcctacatcagttcatccgggtccacaatctactatgcggatagtgtgaaaggtcggttcacaataagtagggataatgcaaagaactcactgtatctccagatgaatagtcttagagccgaagataccgctgtctattattgtgcgcgaatcggtcgatattcatacgggttgagaagctattactattatggcatggacgtttggggccagggtactacagtcactgtatctagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 132 - protein, 119 aa, Artificial Sequence [sequence omitted]

SEQ ID NO: 133 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 134 - protein, 122 aa, Artificial Sequence
  Description of Artificial Sequence Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 135 - protein, 108 aa, Artificial Sequence
  Description of Artificial Sequence Synthetic polypeptide
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 136 - protein, 117 aa, Artificial Sequence
  Description of Artificial Sequence Synthetic polypeptide
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 137 - protein, 108 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 138 - protein, 119 aa, Artificial Sequence
  Description of Artificial Sequence Synthetic polypeptide
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 139 - protein, 107 aa, Artificial Sequence
  Description of Artificial Sequence Synthetic polypeptide
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 140 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 141 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 142 - protein, 114 aa, Artificial Sequence
  Description of Artificial Sequence Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 143 - protein, 114 aa, Artificial Sequence
  Description of Artificial Sequence Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 144 - protein, 107 aa, Artificial Sequence [sequence omitted]

SEQ ID NO: 145 - protein, 114 aa, Artificial Sequence
  Description of Artificial Sequence Synthetic polypeptide
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 146 - protein, 112 aa, Artificial Sequence
  Description of Artificial Sequence Synthetic polypeptide
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 147 - protein, 120 aa, Artificial Sequence
  Description of Artificial Sequence Synthetic polypeptide
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 148 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 149 - protein, 113 aa, Artificial Sequence
  Description of Artificial Sequence Synthetic polypeptide
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 150 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 151 - protein, 182 aa, Homo sapiens
PKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPG

SEQ ID NO: 152 - protein, 8 aa, Homo sapiens
EPKSCDKT

SEQ ID NO: 153 - protein, 271 aa, Artificial Sequence
  Description of Artificial Sequence Synthetic polypeptide
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 154 - DNA, 813 bp, Artificial Sequence
  Description of Artificial Sequence Synthetic polynucleotide
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 155 - protein, 22 aa, Homo sapiens
MDMRVLAQLLGLLLLCFPGARC

SEQ ID NO: 156 - protein, 20 aa, Homo sapiens
MEAPAQLLFLLLLWLPDTTG

SEQ ID NO: 157 - protein, 20 aa, Homo sapiens
MVLQTQVFISLLLWISGAYG

SEQ ID NO: 158 - protein, 19 aa, Homo sapiens
MLPSQLIGFLLLWVPASRG

SEQ ID NO: 159 - protein, 22 aa, Homo sapiens
MDMRVPAQLLGLLLLWLSGARC

SEQ ID NO: 160 - protein, 125 aa, Artificial Sequence
  Description of Artificial Sequence Synthetic polypeptide
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 161 - protein, 107 aa, Artificial Sequence
  Description of Artificial Sequence Synthetic polypeptide
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 162 - protein, 15 aa, Artificial Sequence
  Description of Artificial Sequence Synthetic peptide
GGGGSGGGGSGGGGS

SEQ ID NO: 163 - DNA, 45 bp, Artificial Sequence
  Description of Artificial Sequence Synthetic oligonucleotide
ggaggtggcgggtctggtggaggcggaagcggtggtggcggatcc

SEQ ID NO: 164 - protein, 510 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFGVYYCMQALQTPVTFGQGTRLEIKGGGGSGGGGSGGGGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGRYSYGLRSYYYYGMDVWGQGTTVTVSSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKHHHHHH

SEQ ID NO: 165 - DNA, 1530 bp, Artificial Sequence
  Description of Artificial Sequence Synthetic polynucleotide
gacatccaaatgacgcagagcccgagctcactttcagcctctgtgggagaccgcgtcacgatcacttgccgcgcaagtcagtccattagctcatatcttaattggtaccaacagaaacccggtaaagcaccgaaactgttaatctatgctgccagctcccttcagagcggagtcccttcccgttttagtgggtccggaagtggtacagacttcacactgactatcagttccttacaacctgaagacttcggtgtctattattgtatgcaagcgctgcaaaccccggtgactttcggccaaggaacacgtttggaaatcaagggcggcggaggctcgggaggcgggggcagtggtggtggaggctctcaagtgcagcttgtagaaagtggggggggtctggtgaagccgggggggtcacttcgcctgtcctgtgcagcttcaggatttacattttcggattattatatgagctggattcgccaagctccaggcaagggactggagtgggtctcgtacatctcatcaagcgggagcactatctattatgcagactcggtcaaaggacgtttcaccatttctcgcgataatgccaagaacagcctttacttacaaatgaattcacttcgcgccgaagacacagcagtatattattgtgctcgcatcggtcgttatagttacgggttgcgctcttactattattacgggatggacgtatggggtcagggcactacggtcaccgtcagctcggggggtggtgggagccaggttcagctggtgcaatccggagctgaagtcaaaaagcctggggcaagtgttaaggtttcttgtaaggccagtggttatacatttacgtcatattggatgaactgggtgcgtcaagcgccaggacagggtcttgagtggatgggccgtattgatccttatgattctgagacacattacgcacagaaattacaaggtcgtgtaacgatgacgacagacacatccacctcgaccgcctatatggaattgcgtagcttacgttccgacgatactgcggtgtattattgcgcgcgtggaggttacgatttcgacgttggcactttgtattggttcttcgacgtttggggacaaggaacgacggtaacagtgtcctccgtggagggaggatcagggggtagtggcggctctggcgggagcgggggggtggatgacatccagatgactcagtctcccagtagtttaagtgcttcggtgggagatcgcgtgaccattacctgccgcgcctcggagaatatctactcatatttggcctggtatcaacaaaaaccggggaaagcccccaaattactgatttataacgctaaaacactggccgaaggagtgccgtcacgcttttcagggagcggctccggaactgacttcacattaacaattagttctctgcaaccggaagacttcgccacttactattgtcagcatcattacggtactccacgtaccttcggggggggtactaaggtggagattaagcatcaccaccaccaccat

SEQ ID NO: 166 - protein, 5 aa, Artificial Sequence
  Description of Artificial Sequence Synthetic peptide
SYYMS

SEQ ID NO: 167 - protein, 5 aa, Artificial Sequence
  Description of Artificial Sequence Synthetic peptide
DYAMS

SEQ ID NO: 168 - protein, 5 aa, Artificial Sequence
  Description of Artificial Sequence Synthetic peptide
DYYTS

SEQ ID NO: 169 - protein, 5 aa, Artificial Sequence
  Description of Artificial Sequence Synthetic peptide
DYYMH

SEQ ID NO: 170 - protein, 5 aa, Artificial Sequence
  Description of Artificial Sequence Synthetic peptide
NYYMS

SEQ ID NO: 171 - protein, 5 aa, Artificial Sequence
  Description of Artificial Sequence Synthetic peptide
DNYMS

SEQ ID NO: 172 - protein, 5 aa, Artificial Sequence
  Description of Artificial Sequence Synthetic peptide
DYGMS

SEQ ID NO: 173 - protein, 5 aa, Artificial Sequence
  Description of Artificial Sequence Synthetic peptide
DYYMN

SEQ ID NO: 174 - protein, 5 aa, Artificial Sequence
  Description of Artificial Sequence Synthetic peptide
GYYMS

SEQ ID NO: 175 - protein, 5 aa, Artificial Sequence
  Description of Artificial Sequence Synthetic peptide
DAYMS

SEQ ID NO: 176 - protein, 17 aa, Artificial Sequence
  Description of Artificial Sequence Synthetic peptide
VISSSGSTIYYADSVKG

SEQ ID NO: 177 - protein, 16 aa, Artificial Sequence
  Description of Artificial Sequence Synthetic peptide
YVSSGSTIYYADSVKG

SEQ ID NO: 178 - protein, 17 aa, Artificial Sequence
  Description of Artificial Sequence Synthetic peptide
YIGSSGSTIYYADSVKG

SEQ ID NO: 179 - protein, 17 aa, Artificial Sequence
  Description of Artificial Sequence Synthetic peptide
YISYSGSTIYYADSVKG

SEQ ID NO: 180 - protein, 17 aa, Artificial Sequence
  Description of Artificial Sequence Synthetic peptide
YISSDGSTIYYADSVKG

SEQ ID NO: 181 - protein, 17 aa, Artificial Sequence
  Description of Artificial Sequence Synthetic peptide
YISSSSSTIYYADSVKG

SEQ ID NO: 182 - protein, 17 aa, Artificial Sequence
  Description of Artificial Sequence Synthetic peptide
YISSSGGTIYYADSVKG

SEQ ID NO: 183 - protein, 17 aa, Artificial Sequence
  Description of Artificial Sequence Synthetic peptide
YISSSGSSIYYADSVKG

SEQ ID NO: 184 - protein, 17 aa, Artificial Sequence
  Description of Artificial Sequence Synthetic peptide
YISSSGSTTYYADSVKG

SEQ ID NO: 185 - protein, 17 aa, Artificial Sequence
  Description of Artificial Sequence Synthetic peptide
YISSSGSTIHYADSVKG

SEQ ID NO: 186 - protein, 18 aa, Artificial Sequence
  Description of Artificial Sequence Synthetic peptide
LGRYSYGLRSYYYYGMDV

SEQ ID NO: 187 - protein, 18 aa, Artificial Sequence
  Description of Artificial Sequence Synthetic peptide
ISRYSYGLRSYYYYGMDV

SEQ ID NO: 188 - protein, 18 aa, Artificial Sequence
  Description of Artificial Sequence Synthetic peptide
IGKYSYGLRSYYYYGMDV

SEQ ID NO: 189 - protein, 18 aa, Artificial Sequence
  Description of Artificial Sequence Synthetic peptide
IGRFSYGLRSYYYYGMDV

SEQ ID NO: 190 - protein, 18 aa, Artificial Sequence
  Description of Artificial Sequence Synthetic peptide
IGRYTYGLRSYYYYGMDV

SEQ ID NO: 191 - protein, 18 aa, Artificial Sequence
  Description of Artificial Sequence Synthetic peptide
IGRYSFGLRSYYYYGMDV

SEQ ID NO: 192 - protein, 18 aa, Artificial Sequence
  Description of Artificial Sequence Synthetic peptide
IGRYSYSLRSYYYYGMDV

SEQ ID NO: 193 - protein, 18 aa, Artificial Sequence
  Description of Artificial Sequence Synthetic peptide
IGRYSYGIRSYYYYGMDV

SEQ ID NO: 194 - protein, 18 aa, Artificial Sequence
  Description of Artificial Sequence Synthetic peptide
IGRYSYGLKSYYYYGMDV

SEQ ID NO: 195 - protein, 18 aa, Artificial Sequence
  Description of Artificial Sequence Synthetic peptide
IGRYSYGLRTYYYYGMDV

SEQ ID NO: 196 - protein, 11 aa, Artificial Sequence
  Description of Artificial Sequence Synthetic peptide
QASQSISSYLN

SEQ ID NO: 197 - protein, 11 aa, Artificial Sequence
  Description of Artificial Sequence Synthetic peptide
RMSQSISSYLN

SEQ ID NO: 198 - protein, 11 aa, Artificial Sequence
  Description of Artificial Sequence Synthetic peptide
RARQSISSYLN

SEQ ID NO: 199 - protein, 11 aa, Artificial Sequence
  Description of Artificial Sequence Synthetic peptide
RASESISSYLN

SEQ ID NO: 200 - protein, 11 aa, Artificial Sequence
  Description of Artificial Sequence Synthetic peptide
RASQGISSYLN

SEQ ID NO: 201 - protein, 11 aa, Artificial Sequence
  Description of Artificial Sequence Synthetic peptide
RASQSIRSYLN

SEQ ID NO: 202 - protein, 11 aa, Artificial Sequence
  Description of Artificial Sequence Synthetic peptide
RASQSISNYLN

SEQ ID NO: 203 - protein, 11 aa, Artificial Sequence
  Description of Artificial Sequence Synthetic peptide
RASQSISSWLN

SEQ ID NO: 204 - protein, 11 aa, Artificial Sequence
  Description of Artificial Sequence Synthetic peptide
RASQSISSYLA

SEQ ID NO: 205 - protein, 11 aa, Artificial Sequence
  Description of Artificial Sequence Synthetic peptide
WASQSISSYLN

SEQ ID NO: 206 - protein, 7 aa, Artificial Sequence
  Description of Artificial Sequence Synthetic peptide
DASSLQS

SEQ ID NO: 207 - protein, 7 aa, Artificial Sequence
  Description of Artificial Sequence Synthetic peptide
AAKSLQS

SEQ ID NO: 208 - protein, 7 aa, Artificial Sequence
  Description of Artificial Sequence Synthetic peptide
AASTLQS

SEQ ID NO: 209 - protein, 7 aa, Artificial Sequence
  Description of Artificial Sequence Synthetic peptide
AASSLES

SEQ ID NO: 210 - protein, 7 aa, Artificial Sequence
  Description of Artificial Sequence Synthetic peptide
AASSLQT

SEQ ID NO: 211 - protein, 7 aa, Artificial Sequence
  Description of Artificial Sequence Synthetic peptide
SASSLQS

SEQ ID NO: 212 - protein, 7 aa, Artificial Sequence
  Description of Artificial Sequence Synthetic peptide
AAPSLQS

SEQ ID NO: 213 - protein, 7 aa, Artificial Sequence
  Description of Artificial Sequence Synthetic peptide
AASNLQS

SEQ ID NO: 214 - protein, 7 aa, Artificial Sequence
  Description of Artificial Sequence Synthetic peptide
AASSLHS

SEQ ID NO: 215 - protein, 7 aa, Artificial Sequence
  Description of Artificial Sequence Synthetic peptide
AASSLQP

SEQ ID NO: 216 - protein, 9 aa, Artificial Sequence
  Description of Artificial Sequence Synthetic peptide
QQALQTPVT

SEQ ID NO: 217 - protein, 9 aa, Artificial Sequence
  Description of Artificial Sequence Synthetic peptide
MRALQTPVT

SEQ ID NO: 218 - protein, 9 aa, Artificial Sequence
  Description of Artificial Sequence Synthetic peptide
MQYLQTPVT

SEQ ID NO: 219 - protein, 9 aa, Artificial Sequence
  Description of Artificial Sequence Synthetic peptide
MQANQTPVT

SEQ ID NO: 220 - protein, 9 aa, Artificial Sequence
  Description of Artificial Sequence Synthetic peptide
MQALSTPVT

SEQ ID NO: 221 - protein, 9 aa, Artificial Sequence
  Description of Artificial Sequence Synthetic peptide
MQALQYPVT

SEQ ID NO: 222 - protein, 9 aa, Artificial Sequence
  Description of Artificial Sequence Synthetic peptide
MQALQTPAT

SEQ ID NO: 223 - protein, 9 aa, Artificial Sequence
  Description of Artificial Sequence Synthetic peptide
MQALQTPIT

SEQ ID NO: 224 - protein, 9 aa, Artificial Sequence
  Description of Artificial Sequence Synthetic peptide
MQALQTPLT

SEQ ID NO: 225 - protein, 9 aa, Artificial Sequence
  Description of Artificial Sequence Synthetic peptide
KQALQTPVT

SEQ ID NO: 226 - protein, 16 aa, Artificial Sequence
  Description of Artificial Sequence Synthetic peptide
IARQYRKSYYYYGMDV

SEQ ID NO: 227 - protein, 16 aa, Artificial Sequence
  Description of Artificial Sequence Synthetic peptide
VVRQYRKSYYYYGMDV

SEQ ID NO: 228 - protein, 16 aa, Artificial Sequence
  Description of Artificial Sequence Synthetic peptide
VAKQYRKSYYYYGMDV

SEQ ID NO: 229 - protein, 16 aa, Artificial Sequence
  Description of Artificial Sequence Synthetic peptide
VARNYRKSYYYYGMDV

SEQ ID NO: 230 - protein, 16 aa, Artificial Sequence
  Description of Artificial Sequence Synthetic peptide
VARQFRKSYYYYGMDV

SEQ ID NO: 231 - protein, 16 aa, Artificial Sequence
  Description of Artificial Sequence Synthetic peptide
VARQYKKSYYYYGMDV

SEQ ID NO: 232 - protein, 16 aa, Artificial Sequence
  Description of Artificial Sequence Synthetic peptide
VARQYRRSYYYYGMDV

SEQ ID NO: 233 - protein, 16 aa, Artificial Sequence
  Description of Artificial Sequence Synthetic peptide
VARQYRKTYYYYGMDV

SEQ ID NO: 234 - protein, 16 aa, Artificial Sequence
  Description of Artificial Sequence Synthetic peptide
VARQYRKSFYYYGMDV

SEQ ID NO: 235 - protein, 16 aa, Artificial Sequence
  Description of Artificial Sequence Synthetic peptide
VARQYRKSYFYYGMDV

SEQ ID NO: 236 - protein, 9 aa, Artificial Sequence
  Description of Artificial Sequence Synthetic peptide
QQALQTPLT

SEQ ID NO: 237 - protein, 9 aa, Artificial Sequence
  Description of Artificial Sequence Synthetic peptide
MRALQTPLT

SEQ ID NO: 238 - protein, 9 aa, Artificial Sequence
  Description of Artificial Sequence Synthetic peptide
MQYLQTPLT

SEQ ID NO: 239 - protein, 9 aa, Artificial Sequence
  Description of Artificial Sequence Synthetic peptide
MQANQTPLT

SEQ ID NO: 240 - protein, 9 aa, Artificial Sequence
  Description of Artificial Sequence Synthetic peptide
MQALSTPLT

SEQ ID NO: 241 - protein, 9 aa, Artificial Sequence
  Description of Artificial Sequence Synthetic peptide
MQALQYPLT

SEQ ID NO: 242 - protein, 9 aa, Artificial Sequence
  Description of Artificial Sequence Synthetic peptide
MQALQTSLT

SEQ ID NO: 243 - protein, 9 aa, Artificial Sequence
  Description of Artificial Sequence Synthetic peptide
MQALQTPIT

SEQ ID NO: 244 - protein, 9 aa, Artificial Sequence
  Description of Artificial Sequence Synthetic peptide
KQALQTPLT

SEQ ID NO: 245 - protein, 9 aa, Artificial Sequence
  Description of Artificial Sequence Synthetic peptide
MKALQTPLT

SEQ ID NO: 246 - protein, 6 aa, Artificial Sequence
  Description of Artificial Sequence Synthetic 6xHis tag
HHHHHH

SEQ ID NO: 247 - protein, 18 aa, Artificial Sequence
  Description of Artificial Sequence Synthetic peptide
LGRYSYGLRSYYYYGMDV

SEQ ID NO: 248 - protein, 18 aa, Artificial Sequence
  Description of Artificial Sequence Synthetic peptide
ISRYSYGLRSYYYYGMDV

SEQ ID NO: 249 - protein, 18 aa, Artificial Sequence
  Description of Artificial Sequence Synthetic peptide
IGKYSYGLRSYYYYGMDV

SEQ ID NO: 250 - protein, 18 aa, Artificial Sequence
  Description of Artificial Sequence Synthetic peptide
IGRFSYGLRSYYYYGMDV

SEQ ID NO: 251 - protein, 18 aa, Artificial Sequence
  Description of Artificial Sequence Synthetic peptide
IGRYTYGLRSYYYYGMDV

SEQ ID NO: 252 - protein, 18 aa, Artificial Sequence
  Description of Artificial Sequence Synthetic peptide
IGRYSFGLRSYYYYGMDV

SEQ ID NO: 253 - protein, 18 aa, Artificial Sequence
  Description of Artificial Sequence Synthetic peptide
IGRYSYSLRSYYYYGMDV

SEQ ID NO: 254 - protein, 18 aa, Artificial Sequence
  Description of Artificial Sequence Synthetic peptide
IGRYSYGIRSYYYYGMDV

SEQ ID NO: 255 - protein, 18 aa, Artificial Sequence
  Description of Artificial Sequence Synthetic peptide
IGRYSYGLKSYYYYGMDV

SEQ ID NO: 256 - protein, 18 aa, Artificial Sequence
  Description of Artificial Sequence Synthetic peptide
IGRYSYGLRTYYYYGMDV

(19) United States
(12) Patent Application Publication
(10) Pub. No.: US 2024/0166760 A1
(43) Pub. Date: May 23, 2024

(54) MOLECULES THAT BIND TO CD66e POLYPEPTIDES

(71) Applicant: UNIVERSITY OF PITTSBURGH - OF THE COMMONWEALTH SYSTEM OF HIGHER EDUCATION, Pittsburgh, PA (US)

(72) Inventors: Dimiter Stanchev Dimitrov, Pittsburgh, PA (US); John W. Mellors, Pittsburgh, PA (US); Dusan Baek, Pittsburgh, PA (US)

(21) Appl. No.: 18/281,157

(22) PCT Filed: Mar. 8, 2022
(86) PCT No.: PCT/US2022/019375

§ 371 Date: Sep. 8, 2023

Related U.S. Application Data
(60) Provisional application No. 63/158,205, filed on Mar. 8, 2021.

(51) Int. Cl. C07K 16/30; A61K 39/00; A61K 39/395; A61K 47/68; A61P 35/00; C07K 14/725; C07K 16/28; C12N 5/0783

(52) U.S. Cl. CPC C07K 16/3007; A61K 39/3955; A61K 39/4631; A61K 39/464482; A61K 47/68031; A61K 47/68033; A61K 47/68035; A61K 47/6853; A61P 35/00; C07K 14/7051; C07K 16/2803; C07K 16/2809; C07K 16/283; C07K 16/2851; C12N 5/0636; C07K 2317/622; C07K 2319/02; C07K 2319/03; C07K 2319/30

(57) ABSTRACT

This document provides methods and materials involved in binding a binder (e.g., an antibody, antigen binding fragment, antibody domain, CAR, cell engager, and/or ADC) to a CD66e polypeptide. For example, binders (e.g., antibodies, antigen binding fragments, antibody domains, CARs, cell engagers, and/or ADCs) that bind to a CD66e polypeptide and methods and materials for using one or more such binding molecules to treat a mammal (e.g., a human) having cancer are provided.

### CROSS-REFERENCE TO RELATED APPLICATIONS

This application claims the benefit of U.S. Provisional Application Ser. No. 63/158,205, filed Mar. 8, 2021. The disclosure of the prior application is considered part of (and is incorporated by reference in) the disclosure of this application.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a CD66e polypeptide. For example, this document provides binders (e.g., antibodies, antigen binding fragments, antibody domains, CARs, cell engagers, or ADCs) that bind to a CD66e polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a CD66e polypeptide and methods and materials for using such cells to treat cancer.

### 2. Background Information

CD66e, also known as carcinoembryonic antigen-related cell adhesion molecule 5 (CEACAM5), is a member of the carcinoembryonic antigen (CEA) gene family. CD66e is highly expressed in neuroendocrine prostate cancer, and it contains an N-terminal Ig variable-region-like (IgV) domain and six Ig constant region-type 2-like (IgC2-like) domains, called A1-B1-A2-B2-A3-B3. The A3-B3 domains are regarded as membrane-proximal regions, and these two domains are present in splice variants of CD66e in numerous cancers.

### SUMMARY

This document provides methods and materials involved in binding a molecule (e.g., an antibody, an antigen binding fragment, an antibody domain, a CAR, a cell engager, or an ADC) to a CD66e polypeptide. For example, this document provides binders (e.g., antibodies, antigen binding fragments, antibody domains, CARs, cell engagers, or ADCs) that bind to a CD66e polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a CD66e polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a CD66e polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human CD66e polypeptide as set forth in SEQ ID NO:150 or SEQ ID NO:151 (see, e.g., FIG. 1).

In some cases, two sets of three CDRs of an antigen binding fragment provided herein (e.g., SEQ ID NOs:1-3 and 9-11 or SEQ ID NOs:17-19 and 25-27) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target CD66e⁺ cells (e.g., CD66e⁺ tumor cells and/or CD66e⁺ tumor vasculature), can be engineered into an antibody structure that includes an Fc region to create antibodies having the ability to target CD66e⁺ cells (e.g., CD66e⁺ tumor cells and/or CD66e⁺ tumor vasculature) and induce antibody-dependent cell-mediated cytotoxicity (ADCC) against the target CD66e⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target CD66e⁺ cells (e.g., CD66e⁺ tumor cells and/or CD66e⁺ tumor vasculature) and induce one or more immune responses (e.g., T cell immune responses and/or ADCC using a cell engager in the absence of an Fc-containing antibody) against the target CD66e⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., CD66e⁺ cancer cells) or cancer vasculature (e.g., CD66e⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a CD66e⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to a CD66e polypeptide. For example, cells such as T cells (e.g., CTLs), stem cells (e.g., induced pluripotent stem cells), or NK cells can be engineered to express one or more CARs having the ability to bind to a CD66e polypeptide. Such cells (e.g., CD66e-specific CAR⁺ T cells or NK cells) can be used to treat cancer.

In some cases, a binder (e.g., an antibody, antigen binding fragment, and/or antibody domain) provided herein can be used to detect the presence or absence of a CD66e polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains CD66e⁺ cells (e.g., CD66e⁺ cancer cells). Having the ability to detect the presence or absence of a CD66e polypeptide (e.g., CD66e⁺ cancer cells) can allow clinicians, health professionals, and patients to make better decisions about possible treatment options. For example, detection of CD66e⁺ cancer cells within a mammal can allow clinicians, health professionals, and patients to select an appropriate anti-cancer treatment that targets the CD66e⁺ cancer cells. Such treatments that targets the CD66e⁺ cancer cells can include administration of an anti-CD66e antibody such as SAR408701 and/or one or more of the binders described herein having the ability to bind to a CD66e polypeptide and/or administration of one or more cells (e.g., CD66e-specific CAR⁺ T cells or NK cells) designed to express a binder described herein.

In general, one aspect of this document features an antibody comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody.

In another aspect, this document features an antigen binding fragment comprising (or consisting essentially of or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab.

In another aspect, this document features a chimeric antigen receptor comprising (or consisting essentially of or consisting of) an antigen binding domain, a hinge, a transmembrane domain, and one or more signaling domains, wherein the antigen binding domain comprises an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The antigen binding domain can comprise a scFv having the ability to bind to a CD66e polypeptide. The hinge can comprise a hinge set forth in FIG. 13. The transmembrane domain can comprise a transmembrane domain set forth in FIG. 14. The chimeric antigen receptor can comprise one or more signaling domains set forth in FIG. 15.

In another aspect, this document features a cell comprising a chimeric antigen receptor of the preceding paragraph. The cell can be a T cell, a stem cell, or an NK cell. For example, one aspect of this document features an isolated population of cells, wherein at least one cell of the population comprises a chimeric antigen receptor of the preceding paragraph. The cell can be a T cell, a stem cell, or an NK cell. In some embodiments, at least 50 percent, at least 75 percent, at least 95 percent, at least 99 percent, or 100 percent of the cells of the population can comprise the chimeric antigen receptor.

In another aspect, this document features a cell engager comprising (or consisting essentially of or consisting of) a first antigen binding domain, a linker, and a second antigen binding domain, wherein the first antigen binding domain comprises an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The first antigen binding domain can comprise a scFv having the ability to bind to a CD66e polypeptide. The first antigen binding domain can be an IgG having the ability to bind to a CD66e polypeptide. The linker can comprise a linker set forth in FIG. 10 or FIG. 13. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 18. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 19. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 19.

In another aspect, this document features a nucleic acid (e.g., an isolated nucleic acid) comprising (or consisting essentially of or consisting of) a nucleic acid sequence encoding at least part of an antibody or an antigen-binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The nucleic acid sequence can encode the heavy chain variable domain or region of any one of the (i)-(ii). The nucleic acid sequence can encode the light chain variable domain or region of any one of the (i)-(ii). The nucleic acid can be a viral vector. The nucleic acid can be a phagemid.

In another aspect, this document features a nucleic acid (e.g., an isolated nucleic acid) comprising (or consisting essentially of or consisting of) a nucleic acid sequence encoding a chimeric antigen receptor described above or a cell engager described above. The nucleic acid can be a viral vector. The nucleic acid can be a phagemid.

In another aspect, this document features a host cell comprising a nucleic acid of either of the two preceding paragraphs. For example, one aspect of this document features an isolated population of host cells, wherein at least one host cell of the population comprises a nucleic acid of either of the two preceding paragraphs. In some embodiments, at least 50 percent, at least 75 percent, at least 95 percent, at least 99 percent, or 100 percent of the cells of the population can comprise a nucleic acid of either of the two preceding paragraphs.

In another aspect, this document features a host cell that expresses a chimeric antigen receptor described above or a cell engager described above. The host cell can be a T cell, stem cell, or NK cell. For example, one aspect of this document features an isolated population of host cells, wherein at least one host cell of the population expresses a chimeric antigen receptor described above or a cell engager described above. In some embodiments, at least 50 percent, at least 75 percent, at least 95 percent, at least 99 percent, or 100 percent of the cells of the population can express a chimeric antigen receptor described above or a cell engager described above.

In another aspect, this document features an antibody-drug conjugate (ADC) comprising (or consisting essentially of or consisting of) an antigen binding domain covalently linked to a drug, wherein the antigen binding domain comprises an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The antigen binding domain can comprise a scFv having the ability to bind to a CD66e polypeptide. The antigen binding domain can be an IgG having the ability to bind to a CD66e polypeptide. The drug can be selected from the group consisting of auristatins, mertansine, or pyrrolobenzodiazepine (PBD) dimers.

In another aspect, this document features a composition comprising (or consisting essentially of or consisting of) an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The composition can comprise the antibody. The composition can comprise the antigen binding fragment. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) a cell engager described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) a cell described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of or consisting of) an ADC described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of or consists of) administering, to the mammal, a composition of any of the four preceding paragraphs. The mammal can be a human. The cancer can be a CD66e⁺ cancer. The CD66e⁺ cancer can be selected from the group consisting of CD66e⁺ lung cancer, CD66e⁺ prostate cancer, CD66e⁺ esophageal cancer, CD66e⁺ stomach cancer, CD66e⁺ colorectal cancer, CD66e⁺ liver cancer, CD66e⁺ vaginal cancer, or CD66e⁺ cervical cancer. The number of cancer cells within the mammal can be reduced following the administering step.

In another aspect, this document features a method of treating a mammal having cancer. The method comprises (or consists essentially of or consists of) (a) administering, to the mammal, the composition of any of those same four preceding paragraphs referenced in the preceding paragraph, and (b) administering, to the mammal, a composition comprising a checkpoint inhibitor. The mammal can be a human. The cancer can be a CD66e⁺ cancer. The CD66e⁺ cancer can be selected from the group consisting of CD66e⁺ lung cancer, CD66e⁺ prostate cancer, CD66e⁺ esophageal cancer, CD66e⁺ stomach cancer, CD66e⁺ colorectal cancer, CD66e⁺ liver cancer, CD66e⁺ vaginal cancer, or CD66e⁺ cervical cancer. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells within the mammal can be reduced following the administering steps (a) and (b).

In another aspect, this document features a method for binding a binding molecule to a CD66e polypeptide. The method comprises (or consists essentially of or consists of) contacting the CD66e polypeptide with an antibody or an antigen binding fragment. The antibody can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antibody can comprise the ability to bind to SEQ ID NO:150 or SEQ ID NO:151. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can be a monoclonal antibody. The antibody can be an scFv antibody. The antigen binding fragment can comprise (or consist essentially of or consist of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions). The antigen binding fragment can comprise the ability to bind to SEQ ID NO:150 or SEQ ID NO:151. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can be monoclonal. The antigen binding fragment can be an Fab. The contacting can be performed in vitro. The contacting can be performed in vivo. The contacting can be performed within a mammal by administering the antibody or the antigen binding fragment to the mammal. The mammal can be a human.

In another aspect, this document features a method for binding a binding molecule to a CD66e polypeptide. The method comprises (or consists essentially of or consists of) contacting the CD66e polypeptide with a chimeric antigen receptor described above, a cell engager described above, or an ADC described above. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues 1 to 702 of a human CD66e polypeptide (SEQ ID NO:150). The underlined and bolded amino acid sequence (residues 501 to 682) of this human CD66e polypeptide depicts the A3-B3 domains (SEQ ID NO:151) and was used to identify CD66e binders.

FIGS. 2A and 2B depict the amino acid sequences of the heavy chain variable domain (FIG. 2A) and the light chain variable domain (FIG. 2B) of an Fab designated Clone #1 (ab1). The CDRs, framework sequences, and constant domains of each also are provided and delineated.

FIGS. 3A and 3B depict the amino acid sequences of the heavy chain variable domain (FIG. 3A) and the light chain variable domain (FIG. 3B) of an Fab designated Clone #2 (ab2). The CDRs, framework sequences, and constant domains of each also are provided and delineated.

FIG. 4 depicts the nucleic acid sequences encoding the indicated chains/domains of Clones #1-#2.

FIG. 5 depicts the structure of an exemplary Ig and provides the amino acid and nucleic acid sequences of an exemplary hinge, CH2, and CH3 regions/domains.

FIG. 6A depicts the structure of exemplary scFv's. FIGS. 6B and 6C depict the amino acid sequences of an exemplary heavy chain variable domain (FIG. 6B) and an exemplary light chain variable domain (FIG. 6C) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv. FIGS. 6D-6G depict the structures of exemplary scFv's and provide the amino acid and nucleic acid sequences with the linker, CDRs, and framework sequences delineated.

FIGS. 7A and 7B depict the amino acid sequences of an exemplary heavy chain variable domain (FIG. 7A) and an exemplary light chain variable domain (FIG. 7B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv. FIG. 7C depicts the structures of exemplary scFv's and provides the amino acid and nucleic acid sequences with the linkers, CDRs, and framework sequences delineated.

FIGS. 8A and 8B depict the amino acid sequences of an exemplary heavy chain variable domain (FIG. 8A) and an exemplary light chain variable domain (FIG. 8B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIGS. 9A and 9B depict the amino acid sequences of an exemplary heavy chain variable domain (FIG. 9A) and an exemplary light chain variable domain (FIG. 9B) of an exemplary scFv. The CDRs and framework sequences of each also are delineated. An exemplary linker amino acid sequence such as a linker amino acid sequence set forth in FIG. 10 can be used to link the heavy chain variable domain and the light chain variable domain together to form a scFv.

FIG. 10 depicts exemplary linker amino acid sequences that can be used to link a heavy chain variable domain and a light chain variable domain together to form a scFv. These linker sequences also can be used to create CARs and cell engagers.

FIG. 11A depicts the structure of an exemplary CARs. FIG. 11B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein (e.g., a scFv designed to include two sets of three CDRs such as CDR1, CDR2, and CDR3 of a heavy chain and CDR1, CDR2, and CDR3 of a light chain (in either order) of an antigen binding fragment provided herein, for example, SEQ ID NOs:1-3 and 9-11 or SEQ ID NOs:17-19 and 25-27), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 12 depicts the amino acid sequences of exemplary signal peptides that can be used to design a CAR.

FIG. 13 depicts the amino acid sequences of exemplary hinges that can be used to design a CAR.

FIG. 14 depicts the amino acid sequences of exemplary transmembrane domains that can be used to design a CAR.

FIG. 15 depicts the amino acid sequences of exemplary intracellular signaling domains that can be used to design a CAR.

FIG. 16 depicts an amino acid sequence of a CAR (CAR #1) designed to include a scFv created using the CDRs of the Clone #1 Fab and a nucleic acid sequence encoding that CAR. The various components of this CAR (e.g., domains and linkers) are provided and delineated.

FIG. 17A is a schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G₄S)₃ linker). FIG. 17B depicts an amino acid sequence of a linker sequence (SEQ ID NO:162; nucleic acid sequence of the linker is SEQ ID NO:163) followed by an gOKT3-7 scFv sequence, which can be attached to a light chain as shown in FIG. 17A. FIG. 17B also depicts a nucleic acid sequence encoding that linker and gOKT3-7 scFv.

FIG. 18 depicts the amino acid sequences of exemplary antigen binding domains that can be used to design cell engagers that bind to T cells.

FIG. 19 depicts the amino acid sequences of exemplary antigen binding domains that can be used to design cell engagers that bind to NK cells and the amino acid and nucleic acid sequence for an exemplary BiKE.

FIG. 20A is a schematic depicting the binding of exemplary antibodies provided herein to a CEACAM5 A3B3 domain. FIG. 20B is a schematic depicting one of the rationales of the use of the A3B3 domain as a major antigen for antibody discovery.

FIG. 21. Design of antigen and competitors for panning.

FIG. 22. SDS-PAGE analysis for purified Fc-fused CEACAM5 and CEACAM6 domains.

FIG. 23. Binding of 1G9 Fab to different domains of CEACAM5 in an indirect ELISA.

FIG. 24. Binding of 1G9 Fab and IgG1 to different CEACAM family members and cynomolgus CEACAM5 in indirect ELISA.

FIG. 25. Binding of 1G9 Fab to different domains of CEACAM family members in flow-cytometry analysis.

FIG. 26. Specificity of 1G9 IgG1 in the membrane proteome array (MPA) against >5,800 human membrane associated proteins.

FIG. 27. An equilibrium dissociation constant measurement in surface plasmon resonance (SPR) analysis with Biacore.

FIG. 28. N-linked glycosylation sites on A3B3 domain of CEACAM5 and SDS-PAGE analysis for purified Fc-fused A3B3 domain with Asn to Qln mutations.

FIG. 29. Binding of 1G9 Fab to mutants of A3B3 domain.

FIG. 30. Binding of 1G9 and 1C1 hIgG1 to A3B3 domain of CEACAM5 and AB domain of CEACAM6.

FIG. 31. Binding of 1G9 and 1C1 hIgG1 to mutants of A3B3 domain.

FIG. 32. Competitive ELISA of 1G9 Fab with or without 1C1 IgG1 for binding to CEACAM5.

FIG. 33. Electron-Microscopic (EM) images for the complex of 1G9 or 1C1 hIgG1 with CEACAM5 A3B3 domain.

FIG. 34. Binding of 1G9 and 1C1 hIgG1 to Fc gamma receptors (CD16a, CD65, and CD32).

FIG. 35. Binding of 1G9 and 1C1 hIgG1 to CEACAM5-positive NEPC cell line (NCI-H660), stably CEACAM5-expressing PrAd Du145 cell line (Du145-CEACAM5), and CEACAM5-negative PrAd cell line (Du145).

FIG. 36. ADCC activity with 1G9 and 1C1 hIgG1 against CEACAM5-positive cell lines (NCI-H660 and CEACAM5-positive Du145 cells) and CEACAM5-negative cell line (Du145) in the presence ofNK cells (E:T ratio=5:1).

FIG. 37. ADCC activity with 1G9 and 1C1 hIgG1 against CEACAM5-positive cell line (NCI-H660) in the presence of PBMCs.

FIG. 38. Cell viability assay after treatment of 1G9 hIgG1 in the absence of immune cells in CEACAM5-positive cell line (NCI-H660) and CEACAM5-negative cell line (Du145) for 72 hours.

FIG. 39. Transwell migration assay after treatment of 1G9 hIgG1 in CEACAM5-positive cell line (NCI-H660). Representative images and quantification of migrated cells through the transwell are shown.

FIG. 40. Fluorescence intensity histogram of the remained antibody levels (1G9 and 1C1 hIgG1) on NCI-H660 cell surface after the indicated incubation times at 37° C. and the quantification of mean fluorescence intensity (MFI) for 1G9 and 1C1 hIgG1.

FIG. 41. Design of scFv of 1G9, and the binding of scFv of 1G9 in different VH/VL orientations and in use of linkers to A3B3 domain of CEACAM5.

FIG. 42. Genetic components for exemplary CARs (e.g., 2^{nd} generation and 3^{rd} generation CARs).

FIG. 43. CAR expression on CD4⁺ T and CD8⁺ T population in the control T cells and anti-CEACAM5 CAR-T cells (2^{nd} and 3^{rd} generation CAR-T cells).

FIG. 44. Cytotoxic activity of anti-CEACAM5 CAR-T against CEACAM5-positive cell line (NCI-H660), stably CEACAM5-expressing Du145 cell line (Du145-CEACAM5), and CEACAM5-negative cell line (Du145).

FIG. 45. Structure models for domains of CEACAM5 and CEACAM6 with addition of N-linked glycans.

FIG. 46. Cytokine secretion measured in cell supernatants from the indicated cell cultures.

FIG. 47. Granzyme B, Perforin and IFNγ-positive T cells population (%) in the supernatant at 48 hours (E:T ratio: 2:1) after co-culture of 3^{rd} generation anti-CEACAM5 CAR-T with NCI-H660 cells analyzed by intracellular staining.

FIG. 48. Tumor growth curves and body weight of individual mice for the indicated hIgG1 treatment groups in Du145-CEACAM5 and Du145 tumors.

FIG. 49. Tumor growth curves and body weight of individual mice for the indicated CAR-T treatment groups in Du145-CEACAM5 and Du145 tumors.

FIG. 50. Overall tumor growth inhibition and survival rate after treatment using 1G9 hIgG1 or CAR-T cells.

### DETAILED DESCRIPTION

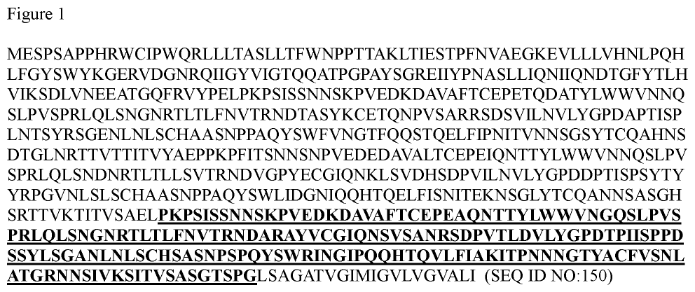

This document provides binders (e.g., antibodies, antigen binding fragments, antibody domains, CARs, cell engagers, and ADCs) that bind (e.g., specifically bind) to a CD66e polypeptide (e.g., a human CD66e polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:150 or SEQ ID NO:151 (see, e.g., FIG. 1). In some cases, a binder (e.g., an antibody, an antigen binding fragment, an antibody domain, a CAR, a cell engager, or an ADC) provided herein can have the ability to bind to a CD66e polypeptide and can lack the ability to bind to a CD66c polypeptide. For example, a binder (e.g., an antibody, an antigen binding fragment, an antibody domain, a CAR, a cell engager, or an ADC) provided herein can have the ability to bind to a human CD66e polypeptide and can lack the ability to bind to a human CD66c polypeptide.

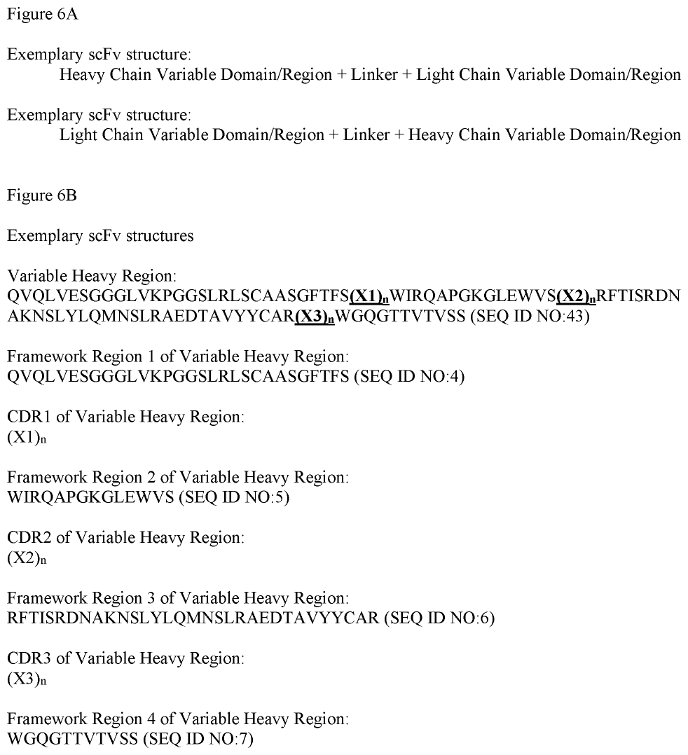

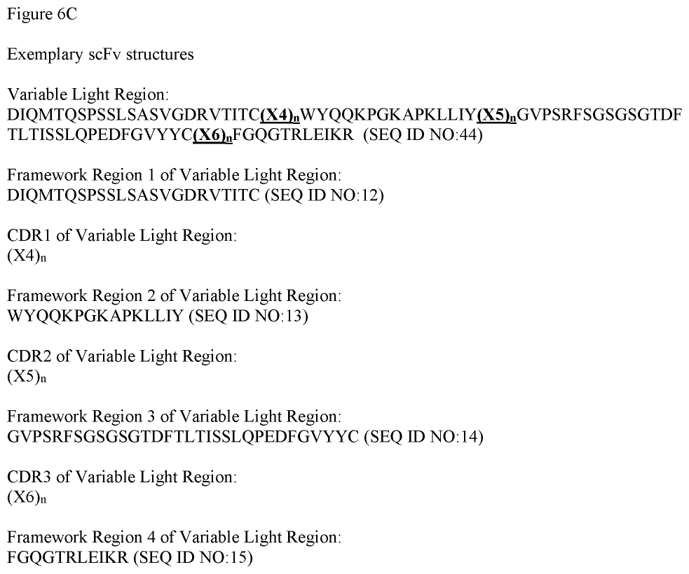

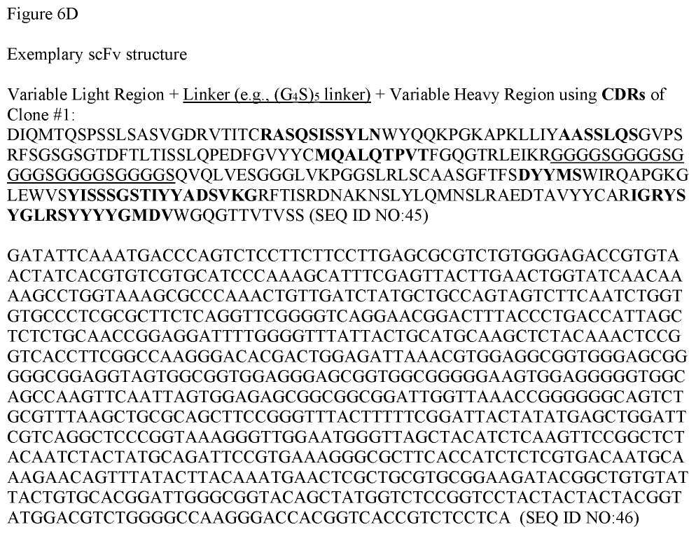

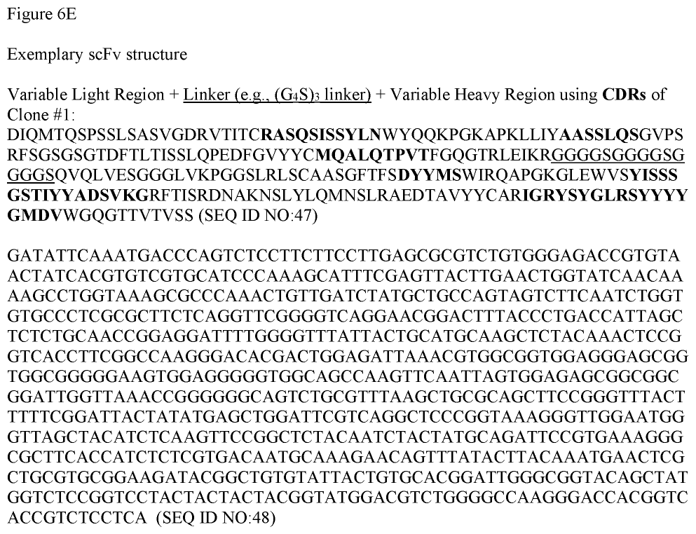

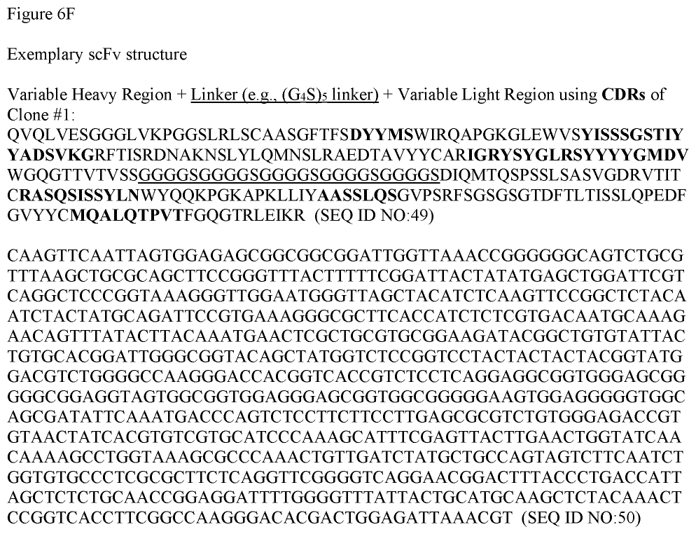

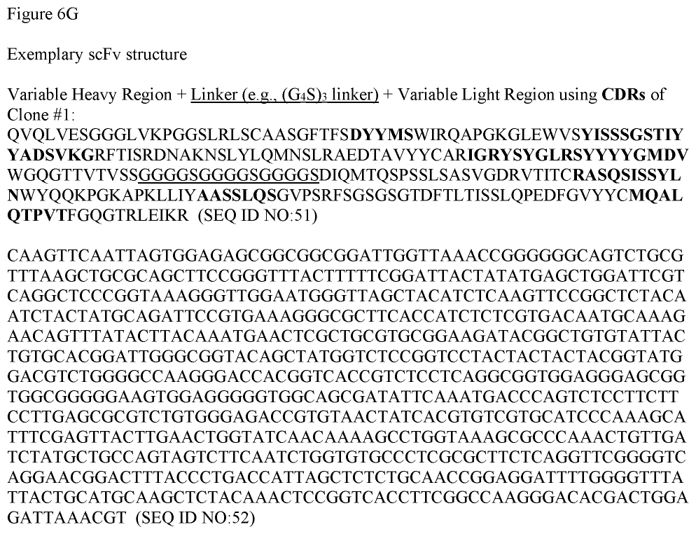

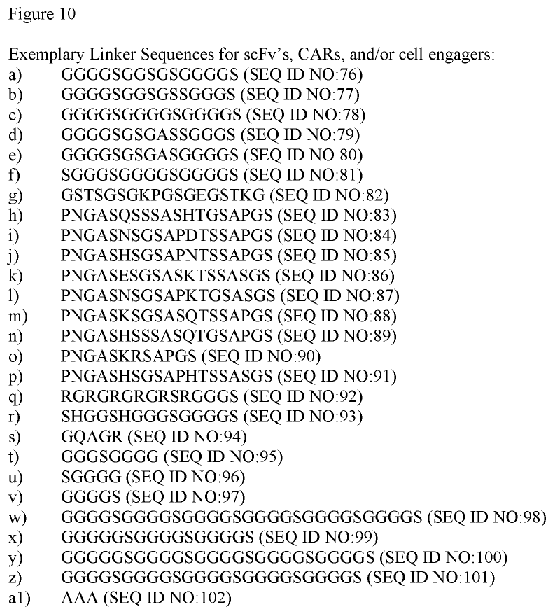

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 10). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 10). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 6A-6Q 7A-7C, 8A-8B, and 9A-9B.

An antibody provided herein can include the CDRs as described herein (e.g., as described in Table 13) and can be configured to be a human antibody, a humanized antibody, or a chimeric antibody. In some cases, an antibody provided herein can include the CDRs as described herein (e.g., as described in Table 13) and can be a monoclonal antibody. In some cases, an antibody provided herein can include the CDRs as described herein (e.g., as described in Table 13) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 13) and can be configured to be a human antigen binding fragment, a humanized antigen binding fragment, or a chimeric antigen binding fragment. In some cases, an antigen binding fragment provided herein can include the CDRs as described herein (e.g., as described in Table 13) and can be a monoclonal antigen binding fragment. In some cases, an antigen binding fragment provided herein can include the CDRs as described herein (e.g., as described in Table 13) and can be configured as an Fab antibody. In some cases, a Fab antibody can include a partial hinge sequence (e.g., SEQ ID NO:152) for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 13) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 13) and can be a monoclonal antibody domain. In some cases, an antibody domain provided herein can include the CDRs as described herein (e.g., as described in Table 13) and can be engineered as a single VH domain or a single VL domain.

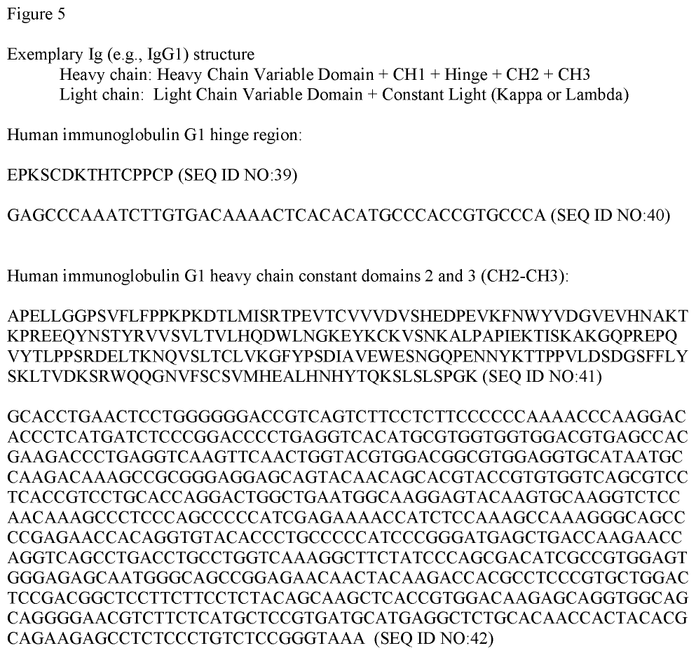

An anti-CD66e antibody, anti-CD66e antigen binding fragment, or anti-CD66e antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-CD66e antibody) can be a scFv antibody. In some cases, an antigen binding fragment provided herein (e.g., an anti-CD66e antibody fragment) can be an Fab. In some cases, an antibody provided herein (e.g., an anti-CD66e antibody) can be a fully intact antibody having the structure set forth in FIG. 5. In some cases, an antibody domain provided herein (e.g., an anti-CD66e antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a CD66e polypeptide (e.g., a human CD66e polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a CD66e polypeptide (e.g., a human CD66e polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a CAR targeting a CD66e polypeptide can be designed to include a VH domain described herein or a scFv antibody described herein.

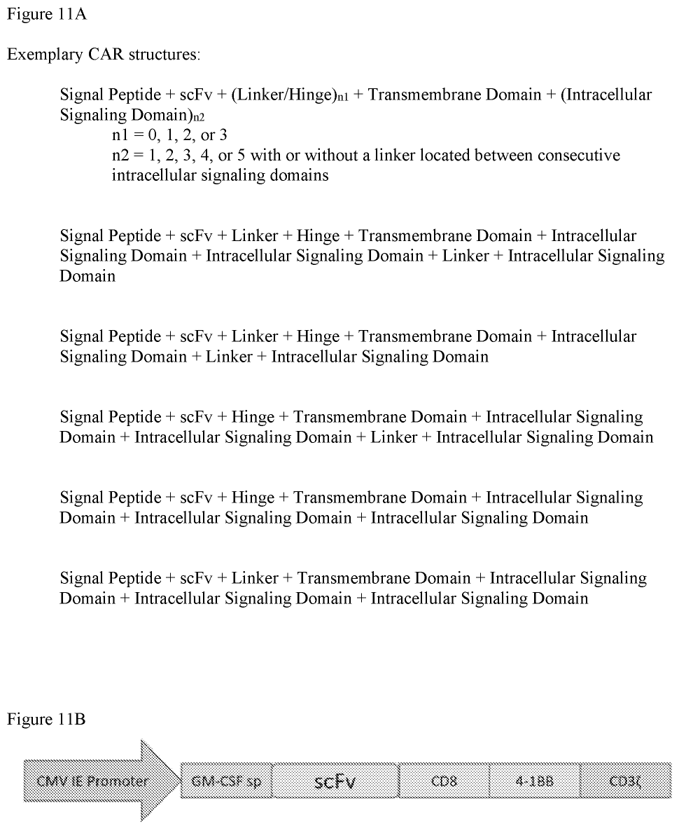

Examples of CAR structures that can be used to make a CAR provided herein include, without limitation, those set forth in FIGS. 11A and 11B.

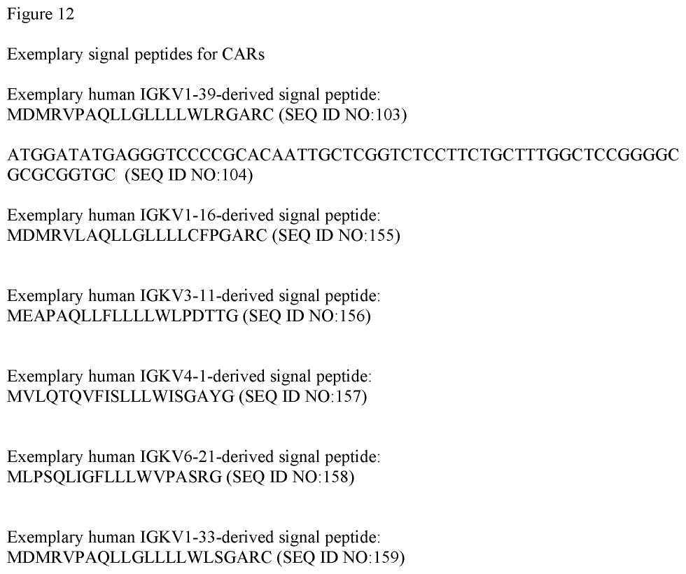

In some cases, a CAR provided herein can be designed to include a signal peptide. Any appropriate signal peptide can be used to design a CAR described herein. Examples of signal peptide that can be used to make a CAR described herein include without limitation, a human IGKV1-39-derived signal peptide, IGKV1-16, IGKV1-33, IGKV3-11, IGKV4-1, or IGKV6-21. In some cases, a CAR provided herein can be designed to include a signal peptide that comprises, consists essentially of, or consists of one of the amino acid sequences set forth in FIG. 12. In some cases, a CAR provided herein can be designed to include a signal peptide that comprises, consists essentially of, or consists of one of the amino acid sequences set forth in FIG. 12 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in FIG. 12 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG₂-derived hinge, or an IgG₄-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in FIG. 10 can be used as a hinge of a CAR described herein.

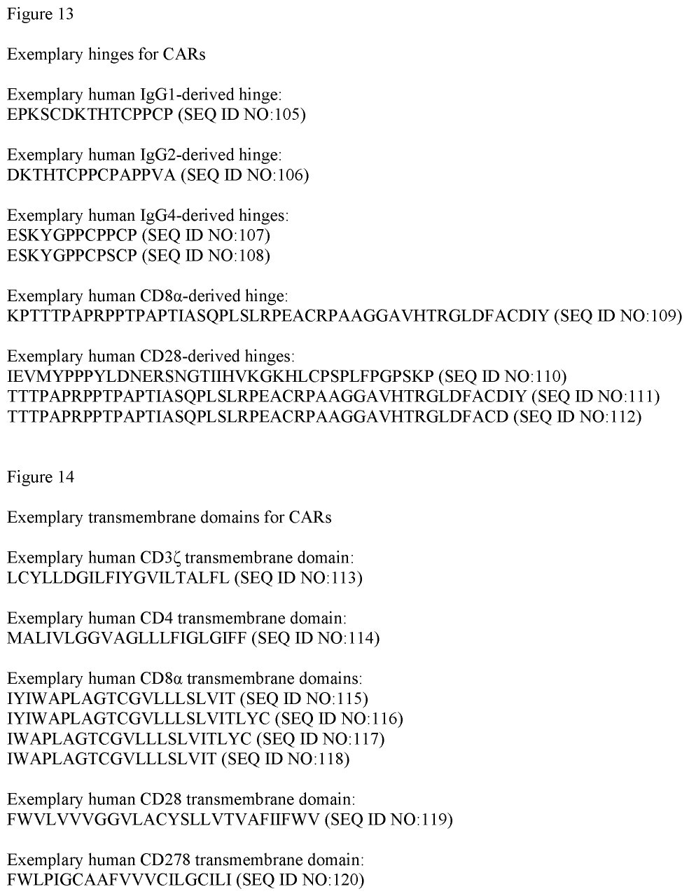

In some cases, a CAR provided herein can be designed to include a hinge that comprises, consists essentially of, or consists of one of the amino acid sequences set forth in FIG. 10 or FIG. 13. In some cases, a CAR provided herein can be designed to include a hinge that comprises, consists essentially of, or consists of one of the amino acid sequences set forth in FIG. 10 or FIG. 13 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 10 or FIG. 13 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. For example, the transmembrane domain of a CAR provided herein can be, without limitation, a CD3ζ transmembrane domain, a CD4 transmembrane domain, a CD8α transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 14. In some cases, a CAR provided herein can be designed to include a transmembrane domain that comprises, consists essentially of, or consists of one of the amino acid sequences set forth in FIG. 14 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 14 with two or less, three or less, four or less, five or less, six or less, seven or less, eight or less, nine or less, or ten or less amino acid deletions, additions, substitutions, or combinations thereof.

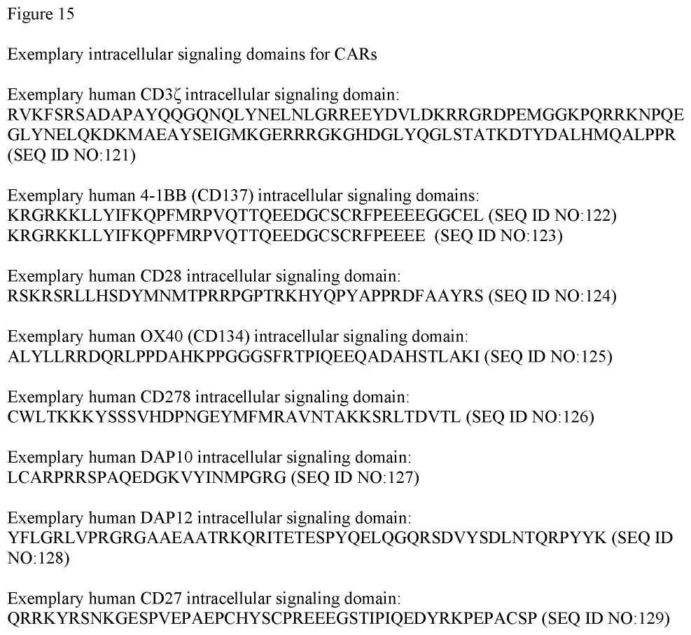

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 15. In some cases, a CAR provided herein can be designed to include at least one intracellular signaling domain that comprises, consists essentially of, or consists of one of the amino acid sequences set forth in FIG. 15 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 15 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a CD66e polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a heavy chain variable domain comprising SEQ ID NO:1, SEQ ID NO:2, and SEQ ID NO:3, followed by SEQ ID NO:100, followed by a light chain variable domain comprising SEQ ID NO:9, SEQ ID NO:10, and SEQ ID NO:11, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

In some cases, a CAR targeting a CD66e polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a heavy chain variable domain comprising SEQ ID NO:8, followed by SEQ ID NO:100, followed by a light chain variable domain comprising SEQ ID NO:16, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

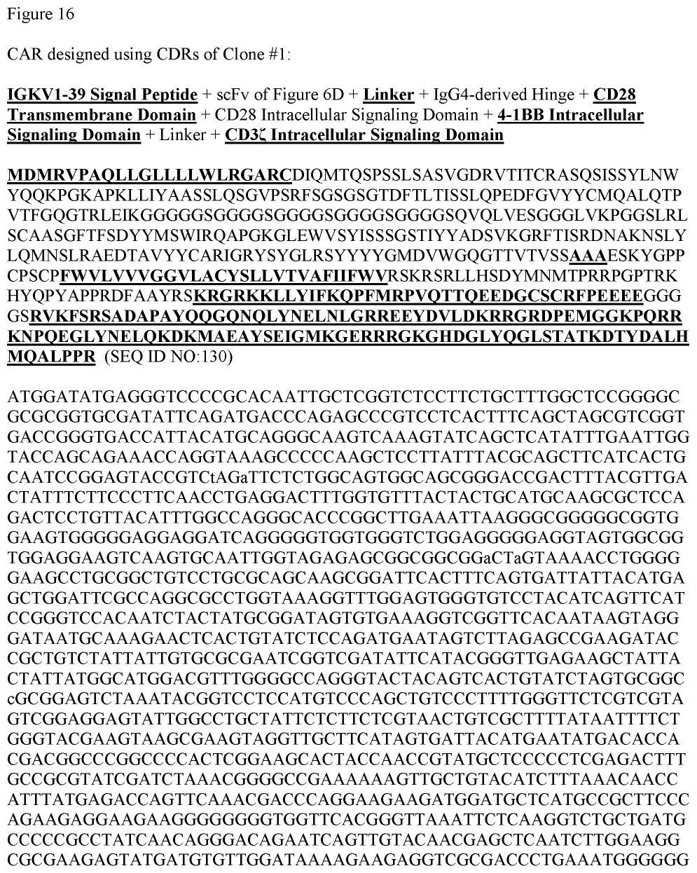

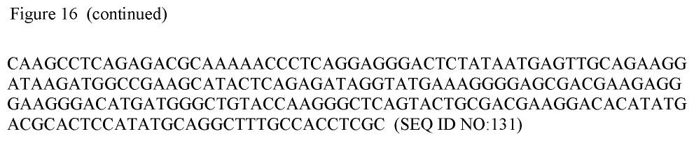

In some cases, a CAR targeting a CD66e polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a light chain variable domain comprising SEQ ID NO:9, SEQ ID NO:10, and SEQ ID NO:11, followed by SEQ ID NO:100, followed by a heavy chain variable domain comprising SEQ ID NO:1, SEQ ID NO:2, and SEQ ID NO:3, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121 (see, e.g., FIG. 16).

In some cases, a CAR targeting a CD66e polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a light chain variable domain comprising SEQ ID NO:16, followed by SEQ ID NO:100, followed by a heavy chain variable domain comprising SEQ ID NO:8, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121 (see, e.g., FIG. 16).

In some cases, a CAR targeting a CD66e polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a heavy chain variable domain comprising SEQ ID NO:17, SEQ ID NO:18, and SEQ ID NO:19, followed by SEQ ID NO:100, followed by a light chain variable domain comprising SEQ ID NO:25, SEQ ID NO:26, and SEQ ID NO:27, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

In some cases, a CAR targeting a CD66e polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a heavy chain variable domain comprising SEQ ID NO:24, followed by SEQ ID NO:100, followed by a light chain variable domain comprising SEQ ID NO:32, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

In some cases, a CAR targeting a CD66e polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a light chain variable domain comprising SEQ ID NO:25, SEQ ID NO:26, and SEQ ID NO:27, followed by SEQ ID NO:100, followed by a heavy chain variable domain comprising SEQ ID NO:17, SEQ ID NO:18, and SEQ ID NO:19, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

In some cases, a CAR targeting a CD66e polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a hinge such as a hinge/linker set forth in FIG. 10 or FIG. 13 (e.g., an IgG₄-derived hinge, a CD8α hinge, or a linker plus IgG₄-derived hinge), followed by a transmembrane domain such as a transmembrane domain set forth in FIG. 14 (e.g., a human CD28 transmembrane domain or a CD8α transmembrane domain), followed by one or more intracellular signaling domains such as one or more intracellular signaling domain set forth in FIG. 15 (e.g., a human 4-1BB intracellular signaling domain followed by a human CD3ζ intracellular signaling domain). For example, a CAR targeting a CD66e polypeptide can be designed to include an scFv having a light chain variable domain comprising SEQ ID NO:32, followed by SEQ ID NO:100, followed by a heavy chain variable domain comprising SEQ ID NO:24, followed by SEQ ID NO:102, followed by SEQ ID NO:108, followed by SEQ ID NO:119, followed by SEQ ID NO:124, followed by SEQ ID NO:123, followed by SEQ ID NO:97, followed by SEQ ID NO:121.

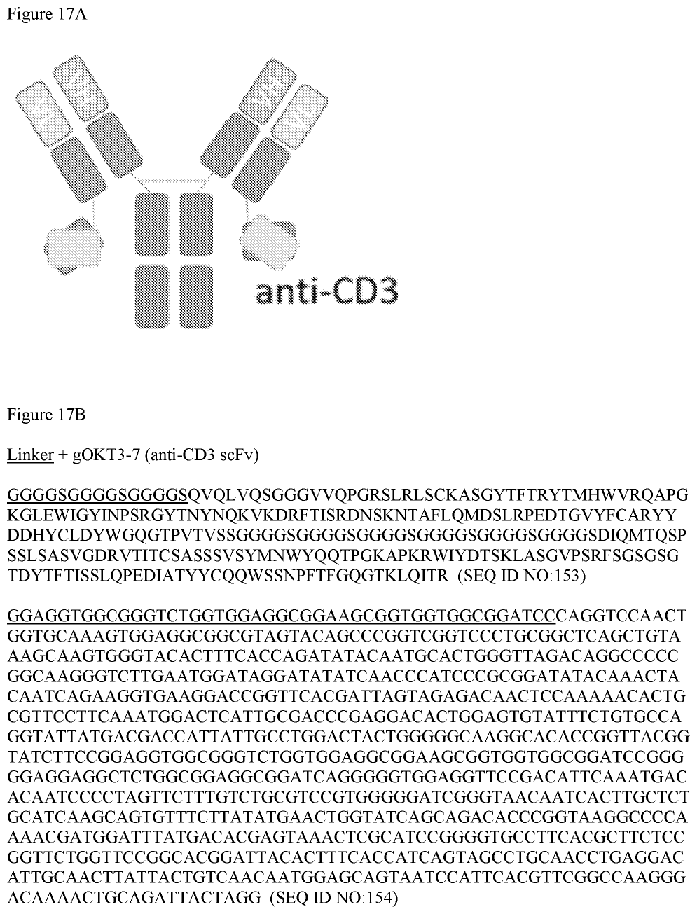

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a CD66e polypeptide (e.g., a human CD66e polypeptide) and at least one antigen binding domain having the ability to bind to an antigen expressed on the surface of a cell (e.g., a T cell or an NK cell). In some cases, a cell engager described herein can link a CD66e⁺ cell (e.g., a CD66e⁺ cancer cell) to another cell (e.g., a T cell or an NK cell) via the two or more antigen binding domains of the cell engager. An example of a cell engager structure of cell engagers provided herein includes, without limitation, the structure set forth in FIG. 17A. In some cases, the anti-CD3 scFv depicted in FIG. 17A can be replace with a different antigen binding domain having the ability to bind to an antigen expressed on the surface of a cell (e.g., a T cell or an NK cell).

When a cell engager includes an antigen binding domain having the ability to bind to a CD66e polypeptide (e.g., a human CD66e polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a CD66e polypeptide (e.g., a human CD66e polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a CD66e polypeptide (e.g., a human CD66e polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a CD66e polypeptide (e.g., a human CD66e polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some cases, an antigen binding domain of a cell engager targeting a CD66e polypeptide can be designed to include a VH domain described herein or a scFv/Fab antibody described herein. In some cases, an antigen binding domain of a CAR described herein that has the ability to bind to a CD66e polypeptide (e.g., a human CD66e polypeptide) can be used as an antigen binding domain of a cell engager that targets CD66e⁺ cells.

As described herein, a cell engager can be designed to include at least one antigen binding domain having the ability to bind to a CD66e polypeptide (e.g., a human CD66e polypeptide) and at least one other antigen binding domain. That at least one other antigen binding domain can have the ability to bind to any appropriate antigen expressed on the surface of a cell. For example, when designing a cell engager such as a BiTE to link a CD66e⁺ cell and a T cell, the cell engager can include an antigen binding domain having the ability to bind to a CD66e polypeptide (e.g., a human CD66e polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

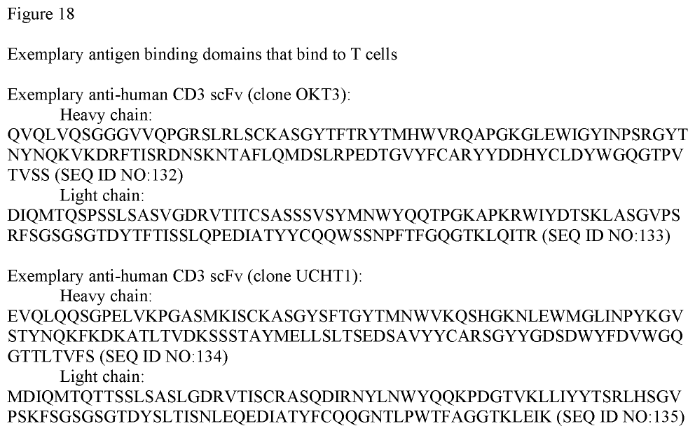

In some cases, a cell engager provided herein can be designed to include an antigen binding domain that comprises, consists essentially of, or consists of one of the amino acid sequences set forth in FIG. 18. In some cases, a cell engager provided herein can be designed to include an antigen binding domain that comprises, consists essentially of, or consists of one of the amino acid sequences set forth in FIG. 18 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 18 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a CD66e⁺ cell and an NK cell, the cell engager can include an antigen binding domain having the ability to bind to a CD66e polypeptide (e.g., a human CD66e polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2Antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

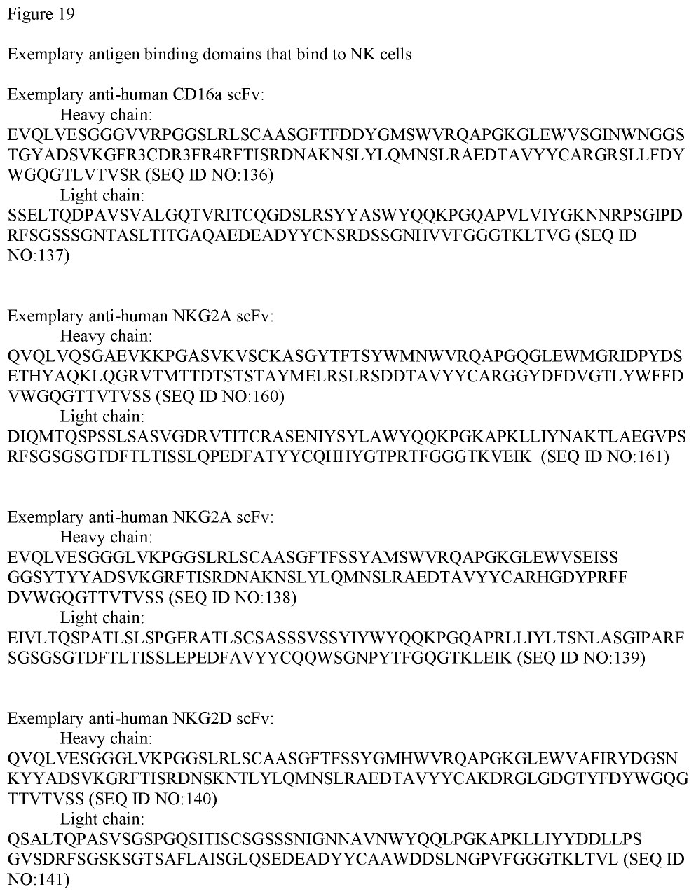

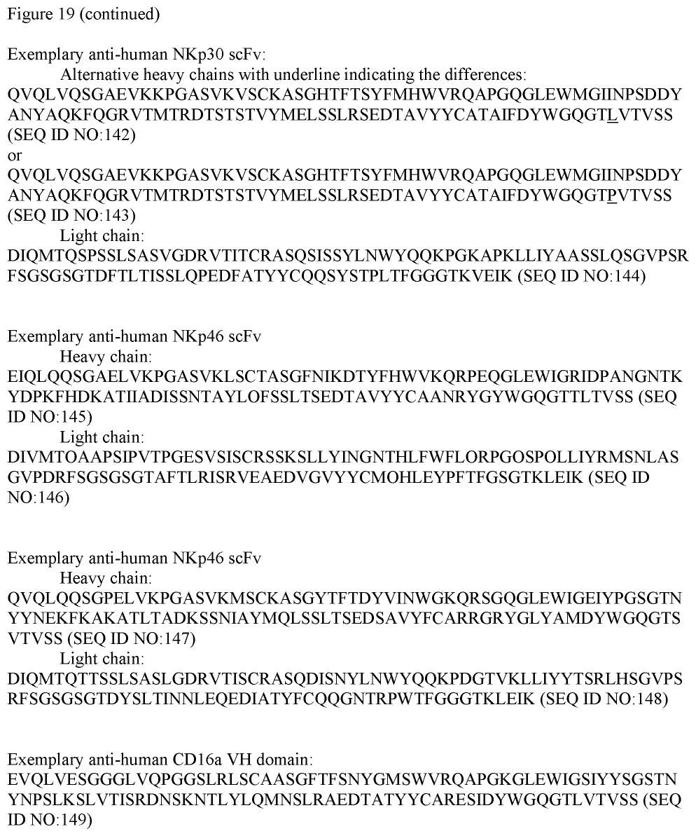

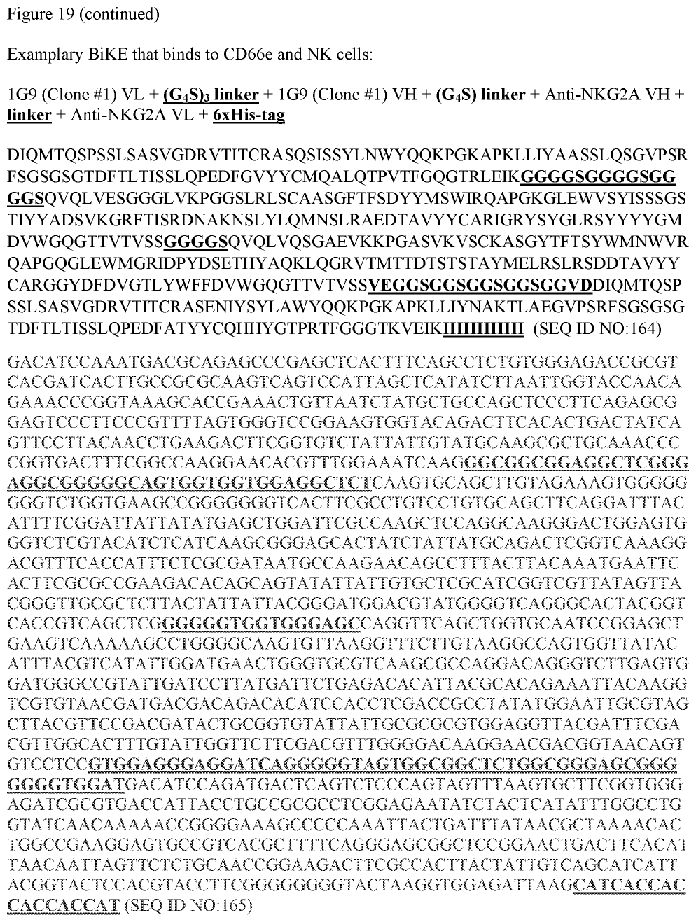

In some cases, a cell engager provided herein can be designed to include an antigen binding domain (e.g., a scFv or VH) that comprises, consists essentially of, or consists of one or more of the amino acid sequences set forth in FIG. 19. In some cases, a cell engager provided herein can be designed to include an antigen binding domain (e.g., a scFv or VH) that comprises, consists essentially of, or consists of one of the amino acid sequences set forth in FIG. 19 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 19 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 10. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:78) linker. In some cases, a hinge of a CAR described herein can be used as a linker to make a cell engager described herein. For example, any one of the sequences set forth in FIG. 13 can be used as a linker of a cell engager described herein.

In some cases, a cell engager provided herein can be designed to include a linker that comprises, consists essentially of, or consists of one of the amino acid sequences set forth in FIG. 10 or FIG. 13. In some cases, a cell engager provided herein can be designed to include a linker that comprises, consists essentially of, or consists of one of the amino acid sequences set forth in FIG. 10 or FIG. 13 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 10 or FIG. 13 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a CD66e polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD66e polypeptide can be designed to include an scFv having a heavy chain variable domain comprising SEQ ID NO:1, SEQ ID NO:2, and SEQ ID NO:3, followed by a linker such as a linker set forth in FIG. 10, followed by a light chain variable domain comprising SEQ ID NO:9, SEQ ID NO:10, and SEQ ID NO:11, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a light chain variable domain comprising SEQ ID NO:9, SEQ ID NO:10, and SEQ ID NO:11, followed by a linker such as a linker set forth in FIG. 10, followed by a heavy chain variable domain comprising SEQ ID NO:1, SEQ ID NO:2, and SEQ ID NO:3, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a heavy chain variable domain comprising SEQ ID NO:8, followed by a linker such as a linker set forth in FIG. 10, followed by a light chain variable domain comprising SEQ ID NO:16, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a light chain variable domain comprising SEQ ID NO:16, followed by a linker such as a linker set forth in FIG. 10, followed by a heavy chain variable domain comprising SEQ ID NO:8, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD66e polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a linker such as a hinge/linker set forth in FIG. 10 or FIG. 13 (e.g., SEQ ID NO:78), followed by an antigen binding domain having the ability to bind to a polypeptide expressed on the surface of a T cell (e.g., an anti-human CD3 scFv).

In some cases, a cell engager (e.g., a BiKE or a TriKE) targeting a CD66e polypeptide can be designed to include an scFv having a heavy chain variable domain comprising SEQ ID NO:17, SEQ ID NO:18, and SEQ ID NO:19, followed by a linker such as a linker set forth in FIG. 10, followed by a light chain variable domain comprising SEQ ID NO:25, SEQ ID NO:26, and SEQ ID NO:27, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a light chain variable domain comprising SEQ ID NO:25, SEQ ID NO:26, and SEQ ID NO:27, followed by a linker such as a linker set forth in FIG. 10, followed by a heavy chain variable domain comprising SEQ ID NO:17, SEQ ID NO:18, and SEQ ID NO:19, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a heavy chain variable domain comprising SEQ ID NO:24, followed by a linker such as a linker set forth in FIG. 10, followed by a light chain variable domain comprising SEQ ID NO:32, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a CD66e polypeptide can be designed to include an scFv having a light chain variable domain comprising SEQ ID NO:32, followed by a linker such as a linker set forth in FIG. 10, followed by a heavy chain variable domain comprising SEQ ID NO:24, followed by a linker such as a hinge/linker set forth in FIG. 10 or FIG. 13 (e.g., SEQ ID NO:78), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a CD66e polypeptide can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a CD66e polypeptide can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a CD66e polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiTE) targeting a CD66e polypeptide can be designed to include an IgG (e.g., IgG1) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a CD66e polypeptide can be designed to include an IgG (e.g., IgG1) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a CD66e polypeptide can be designed to include an IgG (e.g., IgG1) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a CD66e polypeptide can be designed to include an IgG (e.g., IgG1) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

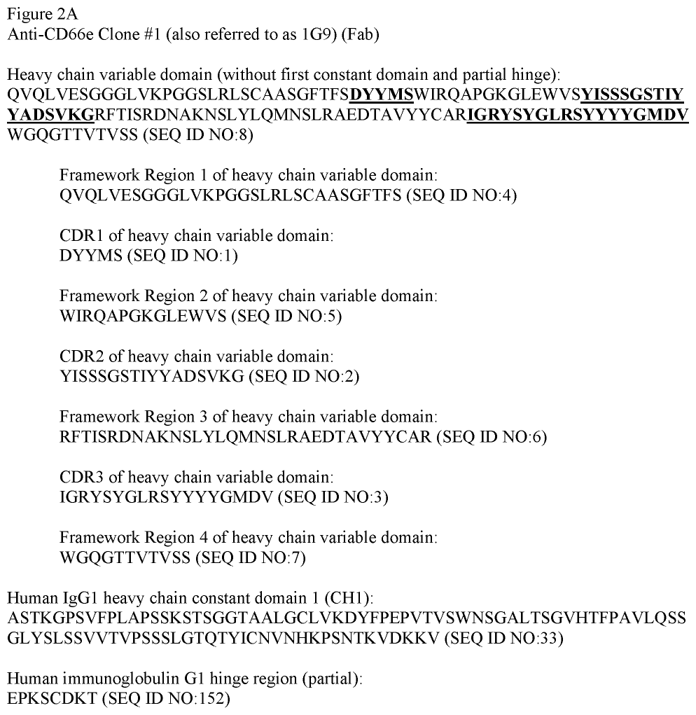

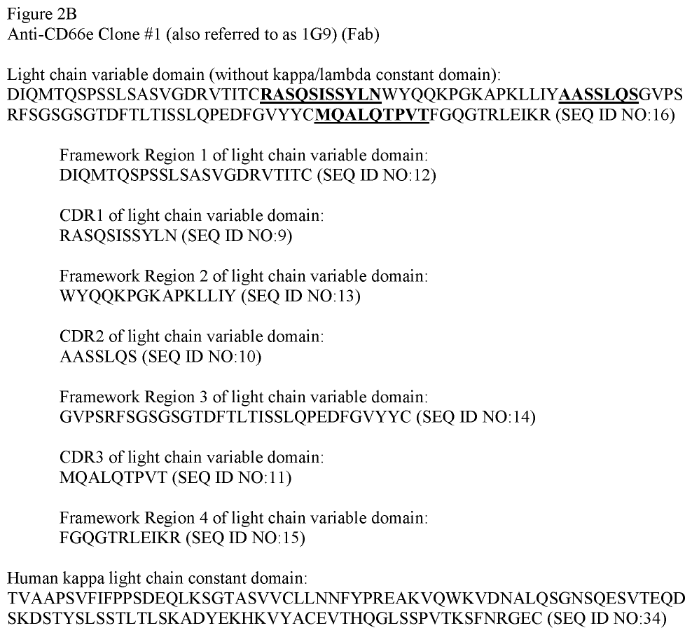

In one embodiment, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications). An example of such an antigen binding fragment having these CDRs and the ability to bind to a CD66e polypeptide (e.g., a human CD66e polypeptide) includes, without limitation, the Fab set forth in FIGS. 2A and 2B.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

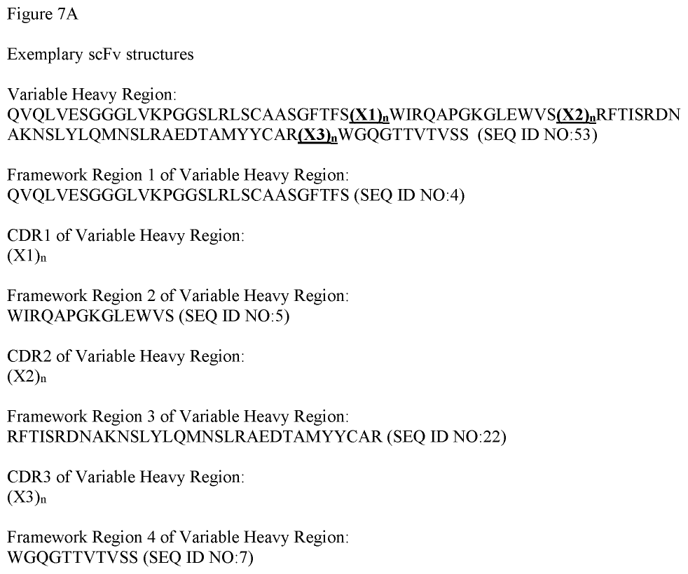

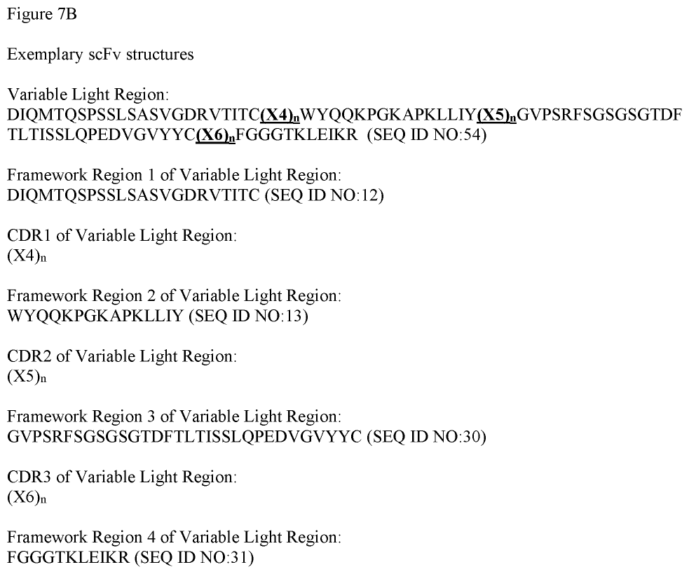

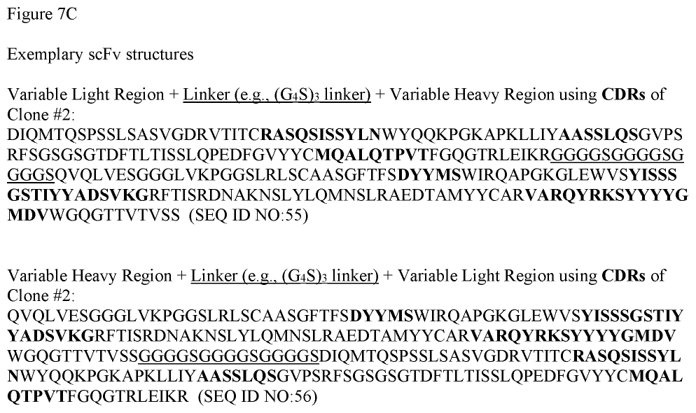

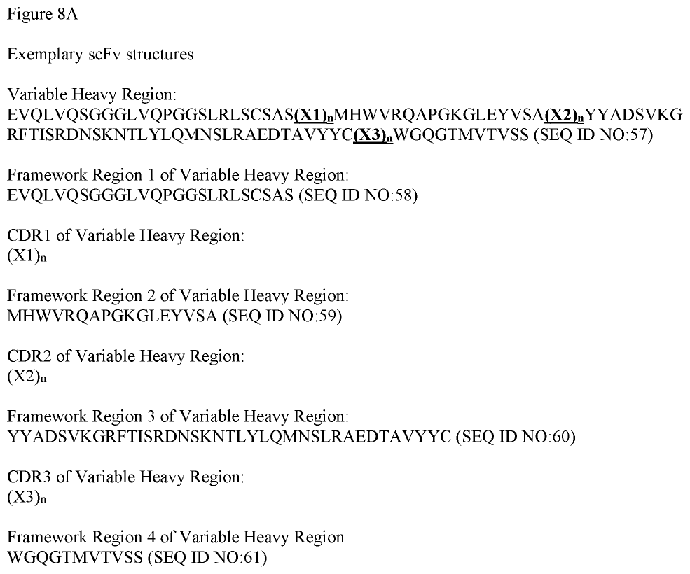

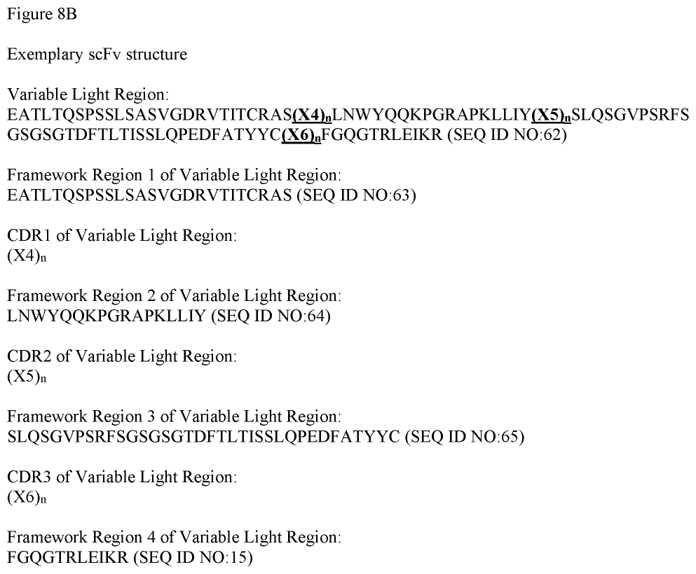

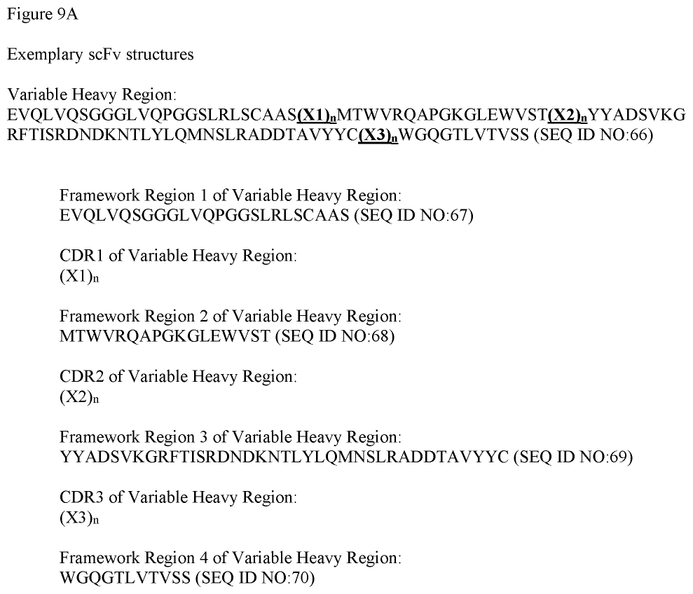

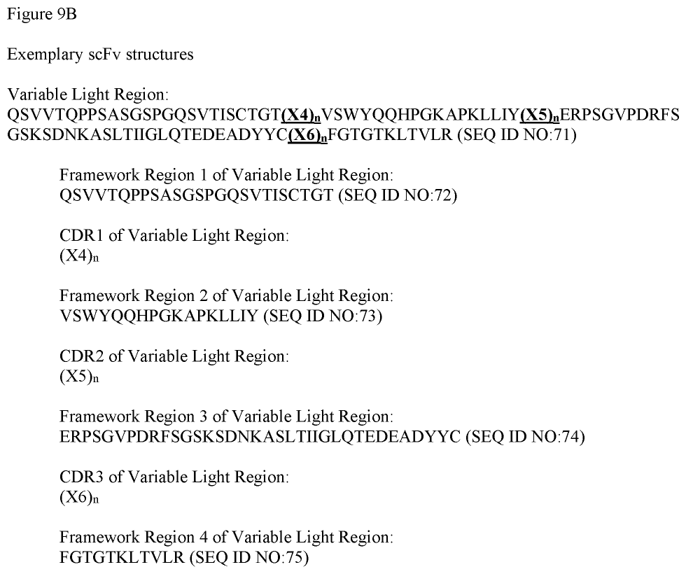

In some cases, a binder (e.g., an antibody, antigen binding fragment, antibody domain, a CAR, a cell engager, and/or an ADC) having any of the CDRs set forth in FIG. 2A or 2B can be designed to include framework regions as set forth in FIGS. 2A and 2B or can be designed to include one or more framework regions from another antibody, antibody fragment, or antibody domain. For example, an Fab can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:58 or a framework region 1 having the amino acid set forth in SEQ ID NO:67 or a framework region 1 having the amino acid set forth in SEQ ID NO:72. In some cases, a scFv can be designed to include the six CDRs set forth in FIGS. 2A and 2B and the framework regions set forth in FIGS. 2A and 2B. In some cases, a scFv can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:58, a framework region 1 having the amino acid set forth in SEQ ID NO:67, or a framework region 1 having the amino acid set forth in SEQ ID NO:72. In another example, a scFv can be designed to include the six CDRs set forth in FIGS. 2A and 2B and the framework regions set forth in FIGS. 6B and 6C, FIGS. 7A and 7B, FIGS. 8A and 8B, or FIGS. 9A and 9B.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD66e polypeptide (e.g., a human CD66e polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR1 that consists essentially of the amino acid sequence set forth in SEQ ID NO:1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| SYYMS | 166 |
| DYAMS | 167 |
| DYYTS | 168 |
| DYYMH | 169 |
| NYYMS | 170 |
| DNYMS | 171 |
| DYGMS | 172 |
| DYYMN | 173 |
| GYYMS | 174 |
| DAYMS | 175 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| VISSSGSTIYYADSVKG | 176 |
| YVSSGSTIYYADSVKG | 177 |
| YIGSSGSTIYYADSVKG | 178 |
| YISYSGSTIYYADSVKG | 179 |
| YISSDGSTIYYADSVKG | 180 |
| YISSSSSTIYYADSVKG | 181 |
| YISSSGGTIYYADSVKG | 182 |
| YISSSGSSIYYADSVKG | 183 |
| YISSSGSTTYYADSVKG | 184 |
| YISSSGSTIHYADSVKG | 185 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| LGRYSYGLRSYYYYGMDV | 186 |
| ISRYSYGLRSYYYYGMDV | 187 |
| IGKYSYGLRSYYYYGMDV | 188 |
| IGRFSYGLRSYYYYGMDV | 189 |
| IGRYTYGLRSYYYYGMDV | 190 |
| IGRYSFGLRSYYYYGMDV | 191 |
| IGRYSYSLRSYYYYGMDV | 192 |
| IGRYSYGIRSYYYYGMDV | 193 |
| IGRYSYGLKSYYYYGMDV | 194 |
| IGRYSYGLRTYYYYGMDV | 195 |

As used herein a “CDR1 that consists essentially of the amino acid sequence set forth in SEQ ID NO:9” is a CDR1 that has zero, one, or two amino acid substitutions within SEQ TD NO:9, that has zero, one, two, three, four, or five amino acid residues directly preceding SEQ ID NO: 9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| QASQSISSYLN | 196 |
| RMSQSISSYLN | 197 |
| RARQSISSYLN | 198 |
| RASESISSYLN | 199 |
| RASQGISSYLN | 200 |
| RASQSIRSYLN | 201 |
| RASQSISNYLN | 202 |
| RASQSISSWLN | 203 |
| RASQSISSYLA | 204 |
| WASQSISSYLN | 205 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

| Sequence | SEQ ID NO: |
|---|---|
| DASSLQS | 206 |
| AAKSLQS | 207 |
| AASTLQS | 208 |
| AASSLES | 209 |
| AASSLQT | 210 |
| SASSLQS | 211 |
| AAPSLQS | 212 |
| AASNLQS | 213 |
| AASSLHS | 214 |
| AASSLQP | 215 |

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| QQALQTPVT | 216 |
| MRALQTPVT | 217 |
| MQYLQTPVT | 218 |
| MQANQTPVT | 219 |
| MQALSTPVT | 220 |
| MQALQYPVT | 221 |
| MQALQTPAT | 222 |
| MQALQTPIT | 223 |
| MQALQTPLT | 224 |
| KQALQTPVT | 225 |

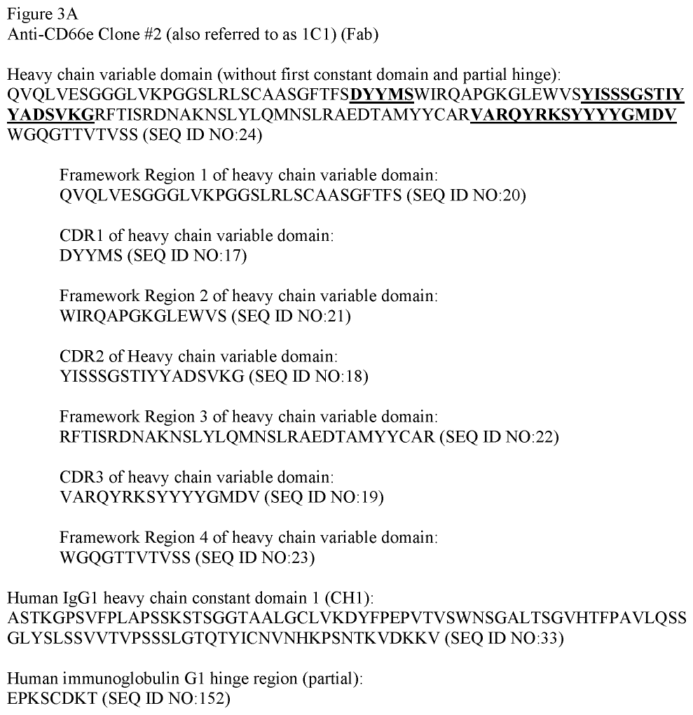

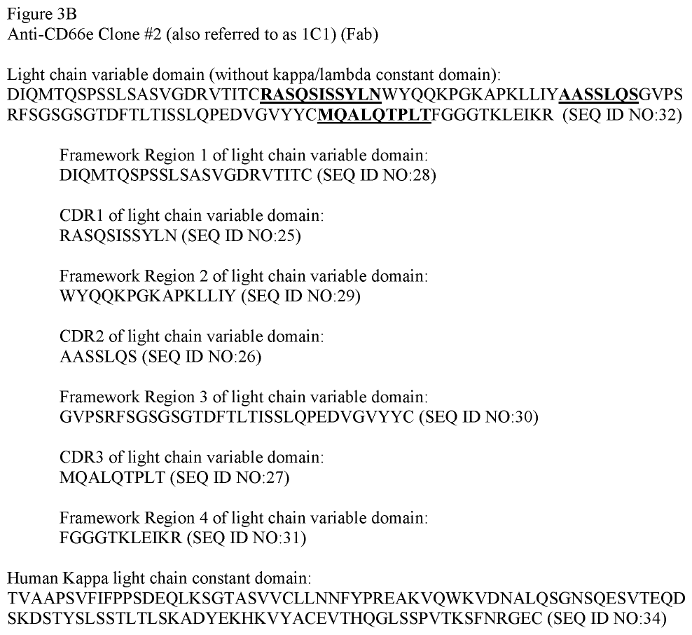

In another embodiment, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (i) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications); and/or (ii) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications). An example of such an antigen binding fragment having these CDRs and the ability to bind to a CD66e polypeptide (e.g., a human CD66e polypeptide) includes, without limitation, the Fab set forth in FIGS. 3A and 3B.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) and having (a) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) and/or (b) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 3A or 3B can be designed to include framework regions as set forth in FIGS. 3A and 3B or can be designed to include one or more framework regions from another antibody or antibody fragment. For example, an Fab can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:58 or a framework region 1 having the amino acid set forth in SEQ ID NO:67 or a framework region 1 having the amino acid set forth in SEQ ID NO:72. In some cases, a scFv can be designed to include the six CDRs set forth in FIGS. 3A and 3B and the framework regions set forth in FIGS. 3A and 3B. In some cases, a scFv can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:58, a framework region 1 having the amino acid set forth in SEQ ID NO:67, or a framework region 1 having the amino acid set forth in SEQ ID NO:72. In another example, a scFv can be designed to include the six CDRs set forth in FIGS. 3A and 3B and the framework regions set forth in FIGS. 6B and 6C, FIGS. 7A and 7B, FIGS. 8A and 8B, or FIGS. 9A and 9B.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a CD66e polypeptide (e.g., a human CD66e polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a CD66e polypeptide (e.g., a human CD66e polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR1 that consists essentially of the amino acid sequence set forth in SEQ ID NO:17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| SYYMS | 166 |
| DYAMS | 167 |
| DYYTS | 168 |
| DYYMH | 169 |
| NYYMS | 170 |
| DNYMS | 171 |
| DYGMS | 172 |
| DYYMN | 173 |
| GYYMS | 174 |
| DAYMS | 175 |

As used herein, a “CDR2 that consists essentially of the amino acid sequence set forth in SEQ ID NO: 18” is a CDR2 that has zero, one, or two amino acid substitutions within SEQ TD NO: 18, that has zero, one, two, three, four, or five amino acid residues directly preceding SEQ ID NO:18, and/or that has zero, one, two, three, four, or five amino acid residues directly following SEQ ID NO: 18, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR2 that consists essentially of the amino acid sequence set forth in SEQ ID NO: 18 include, without limitation, those set forth in Table 8.

TABLE 8
Exemplary CDR2s that consist essentially of the
amino acid sequence set forth in SEQ ID NO: 18.

| Sequence | SEQ ID NO: |
|---|---|
| LGRYSYGLRSYYYYGMDV | 176 |
| ISRYSYGLRSYYYYGMDV | 177 |
| IGKYSYGLRSYYYYGMDV | 178 |
| IGRFSYGLRSYYYYGMDV | 179 |
| IGRYTYGLRSYYYYGMDV | 180 |
| IGRYSFGLRSYYYYGMDV | 181 |
| IGRYSYSLRSYYYYGMDV | 182 |
| IGRYSYGIRSYYYYGMDV | 183 |
| IGRYSYGLKSYYYYGMDV | 184 |
| IGRYSYGLRTYYYYGMDV | 185 |

As used herein, a “CDR3 that consists essentially of the amino acid sequence set forth in SEQ ID NO: 19” is a CDR3 that has zero, one, or two amino acid substitutions within SEQ TD NO: 19, that has zero, one, two, three, four, or five amino acid residues directly preceding SEQ ID NO: 19, and/or that has zero, one, two, three, four, or five amino acid residues directly following SEQ ID NO: 19, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR3 that consists essentially of the amino acid sequence set forth in SEQ ID NO: 19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| IARQYRKSYYYYGMDV | 226 |
| VVRQYRKSYYYYGMDV | 227 |
| VAKQYRKSYYYYGMDV | 228 |
| VARNYRKSYYYYGMDV | 229 |
| VARQFRKSYYYYGMDV | 230 |
| VARQYKKSYYYYGMDV | 231 |
| VARQYRRSYYYYGMDV | 232 |
| VARQYRKTYYYYGMDV | 233 |
| VARQYRKSFYYYGMDV | 234 |
| VARQYRKSYFYYGMDV | 235 |

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR1 that consists essentially of the amino acid sequence set forth in SEQ ID NO:25 include, without limitation, those set forth in Table 10.

TABLE 10
Exemplary CDR1s that consist essentially of the
amino acid sequence set forth in SEQ ID NO: 25.

| Sequence | SEQ ID NO: |
|---|---|
| QASQSISSYLN | 196 |
| RMSQSISSYLN | 197 |
| RARQSISSYLN | 198 |
| RASESISSYLN | 199 |
| RASQGISSYLN | 200 |
| RASQSIRSYLN | 201 |
| RASQSISNYLN | 202 |
| RASQSISSWLN | 203 |
| RASQSISSYLA | 204 |
| WASQSISSYLN | 205 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR2 that consists essentially of the amino acid sequence set forth in SEQ ID NO:26 include, without limitation, those set forth in Table 11.

TABLE 11
Exemplary CDR2s that consist essentially of the
amino acid sequence set forth in SEQ ID NO: 26.

| Sequence | SEQ ID NO: |
|---|---|
| DASSLQS | 206 |
| AAKSLQS | 207 |
| AASTLQS | 208 |
| AASSLES | 209 |
| AASSLQT | 210 |
| SASSLQS | 211 |
| AAPSLQS | 212 |
| AASNLQS | 213 |
| AASSLHS | 214 |
| AASSLQP | 215 |

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a CD66e polypeptide (e.g., a human CD66e polypeptide). Examples of a CDR3 that consists essentially of the amino acid sequence set forth in SEQ ID NO:27 include, without limitation, those set forth in Table 12.

TABLE 12
Exemplary CDR3s that consist essentially of the
amino acid sequence set forth in SEQ ID NO: 27.

| Sequence | SEQ ID NO: |
|---|---|
| QQALQTPLT | 236 |
| MRALQTPLT | 237 |
| MQYLQTPLT | 238 |
| MQANQTPLT | 239 |
| MQALSTPLT | 240 |
| MQALQYPLT | 241 |
| MQALQTSLT | 242 |
| MQALQTPIT | 243 |
| KQALQTPLT | 244 |
| MKALQTPLT | 245 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3 or SEQ ID NOs:17-19 can be directly connected to a light chain variable domain having the CDRs of SEQ ID NOs:9-11 or SEQ ID NOs:25-27, respectively, via a linker sequence. Examples of linker sequences that can be used to connect a heavy chain variable domain and a light chain variable domain to create a scFv include, without limitation, those linkers set forth in FIG. 10.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a CD66e polypeptide (e.g., a human CD66e polypeptide) are further described in Table 13.

TABLE 13
Representative number of binders.

| | SEQ ID | SEQ ID | | SEQ ID | SEQ ID | |
|---|---|---|---|---|---|---|
| | NOs of | NOs of | SEQ ID | NOs of | NOs of | SEQ ID |
| | Heavy | Heavy Chain | NO of | Light | Light Chain | NO of |
| | Chain | Variable | Heavy | Chain | Variable | Light |
| | Variable | Domain/ | Chain | Variable | Domain/ | Chain |
| Clone # | Domain/ | Region | Variable | Domain/ | Region | Variable |
| (Antibody | Region | Framework | Domain/ | Region | Framework | Domain/ |
| type) | CDRs | Regions | Region | CDRs | Regions | Region |
| #1 (Fab) | 1, 2, 3 | 4, 5, 6, 7 | 8 | 9, 10, 11 | 12, 13, 14, 15 | 16 |
| #2 (Fab) | 17, 18, 19 | 20, 21, 22, 23 | 24 | 25, 26, 27 | 28, 29, 30, 31 | 32 |

Table 14 includes an alternative designation that can be used to refer to each of Clones #1-#2.

TABLE 14
Alternative nomenclature for Clones #1-#2.

| Clone # | Alternative names |
|---|---|
| 1 | ab1 |
| 2 | ab2 |

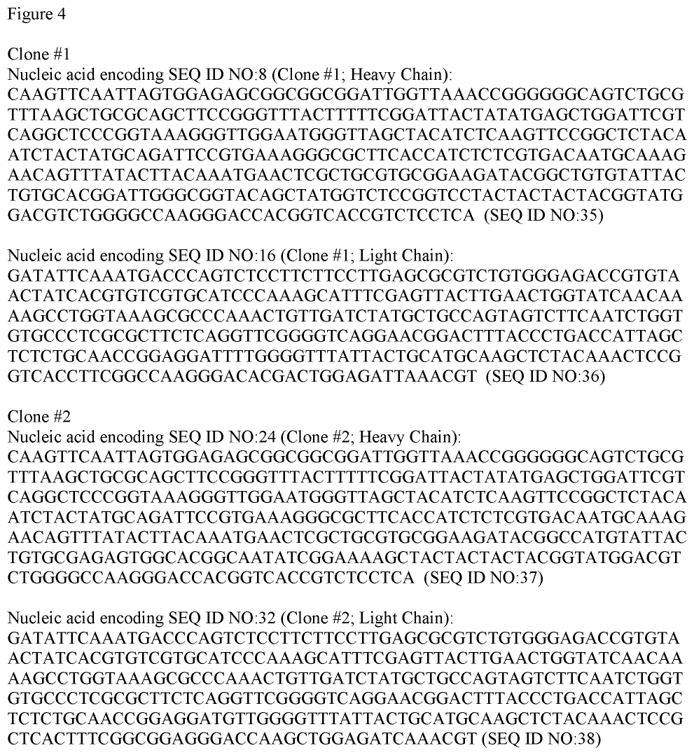

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 4 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a CD66e polypeptide (e.g., a human CD66e polypeptide). In some cases, a bispecific binder provided herein can be designed to bind to two different epitopes of the same CD66e polypeptide (e.g., a human CD66e polypeptide). In some cases, a bispecific binder provided herein can bind to a CD66e polypeptide (e.g., a human CD66e polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a CD66e polypeptide (e.g., a human CD66e polypeptide) and (b) at least one drug covalently linked directly or indirectly to that antigen binding domain. Any appropriate binder (e.g., an antibody, antigen binding fragment, and/or antibody domain) provided herein and having the ability to bind to a CD66e polypeptide (e.g., a human CD66e polypeptide) can be used as an antigen binding domain to make an ADC described herein. For example, any of the binders set forth in Table 13 can be used to make an ADC having the ability to bind to a CD66e polypeptide (e.g., a human CD66e polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a CD66e polypeptide (e.g., a human CD66e polypeptide) to form an ADC provided herein. For example, cleavable or non-cleavable ADC linkers can be used to covalently attach one or more drugs to an antigen binding domain having the ability to bind to a CD66e polypeptide (e.g., a human CD66e polypeptide) to form an ADC provided herein. Examples of ADC linkers can be used to covalently attach one or more drugs to an antigen binding domain having the ability to bind to a CD66e polypeptide (e.g., a human CD66e polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in FIG. 2A or 3A. In another example, an isolated nucleic acid molecule provided herein can include a nucleic acid sequence encoding a light chain variable domain such as a light chain variable domain as set forth in FIG. 2B or 3B. In some cases, an isolated nucleic acid molecule provided herein can include a nucleic acid sequence encoding both (a) a heavy chain variable domain and (b) a light chain variable domain, with or without, encoding a linker polypeptide set forth in FIG. 10. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, N Y (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokayotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukayotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, N Y (1989); Davis et al., Basic Methods in Molecular Biology (1986); and Neumann et al., EMBO J, 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a CD66e polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a CD66e polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a CD66e polypeptide (e.g., a human CD66e polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a CD66e polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a CD66e polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, lung cancer, prostate cancer, esophageal cancer, stomach cancer, colorectal cancer, liver cancer, vaginal cancer, cervical cancer, pancreatic cancer, and breast cancer. In some cases, a mammal (e.g., a human) having a CD66e⁺ cancer (e.g., a CD66e⁺ lung cancer, a CD66e⁺ prostate cancer, a CD66e⁺ esophageal cancer, a CD66e⁺ stomach cancer, a CD66e⁺ colorectal cancer, a CD66e⁺ liver cancer, a CD66e⁺ vaginal cancer, or a CD66e⁺ cervical cancer) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a CD66e polypeptide (e.g., a human CD66e polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a CD66e polypeptide (e.g., a human CD66e polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a CD66e polypeptide (e.g., a human CD66e polypeptide). In some cases, a binder (e.g., an antibody, antigen binding fragment, and/or antibody domain) provided herein can be immobilized, e.g., on a support, and retention of a CD66e polypeptide (e.g., a human CD66e polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a CD66e polypeptide (e.g., a human CD66e polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human CD66e Polypeptide

CD66e contains an N-terminal Ig variable-region-liken (IgV) domain and six Ig constant region-type 2-like (IgC2-like) domains, N-A1-B1-A2-B2-A3-B3 joined to a GPI-anchor to the plasma membrane. Notably, the membrane-proximal A3B3 domains are found in splice variants of CD66e in numerous cancers so they were considered potent therapeutic epitopes. Here, two Fab antibody fragments (Clones: #1 and #2; FIGS. 2 and 3) were identified. To identify these Fabs, Fc-fused soluble CEACAM5 and CEACAM6 domains were designed and applied to a panning process. Isolated Fabs bound A3B3 domains and dominantly bound B3 domain and N-linked glycans at N612 and N650 of CEACAM5 in high affinity and specificity. Moreover, they did not cross-reactive to either other domains of CEACAM5 or CEACAM family members.

The sequences of Clone #1 were used to make an hIgG1 that exhibited CEACAM5-dependent cytotoxic activity in ADCC assays with the NEPC cell line, NCI-H660, and the PrAd cell line, Du145 in the presence of either primary NK cells or PBMCs. Moreover, constructed third generation CARs containing an scFv having the CDRs of Clone #1 were delivered to T cells that were shown to efficiently kill CEACAM5 positive prostate cancer cells while no detectable cytotoxicity was founded in CEACAM5 negative cells. These results demonstrate an immunotherapeutic potential of Clone #1 hIgG1 for NEPC treatment with considerably low off-target toxicity.

### Preparation of Soluble Fc-Fused Recombinant CEACAM5 and CEACAM6 Domains

Human immunoglobulin 1 Fc region (hIgG1 Fc) fused CEACAM5 A3B3 (residues 501-682), CEACAM5 A1B1 (residues 145-322), CEACAM5 A2B2 (residues 323-500), and CEACAM6 AB (residues 145-296) were synthesized and then cloned into pSectag2A plasmid (Invitrogen, V90020). Each plasmid DNA was complexed with PEI-Max (Polysciences, 24765-1) and supplied to culture of the Freestyle human embryonic kidney cell-line (Gibco, R79007) for the transient transfection. Seven days post-transfection, Fc-fused recombinant proteins were purified by affinity chromatography with protein A resin (Captiva, NC0997253). Elution of bound proteins to protein A was eluted by adding 50 mM Glycine buffer pH 3.0, and then storage buffer was changed to phospho-buffered saline pH 7.4 (PBS) by using PD-10 desalting column (GE, 45-000-148). Protein purity was estimated in either SDS-PAGE or size exclusion chromatography packed with Superdex 200 increase 10/300 GL (GE healthcare, 28990944). The concentration of each proteins was determined by Nano Drop spectrophotometer 2000C (Thermo, ND2000C). N-glycosylation mutants of A3B3 domain of CEACAM5 (N508Q, N529Q, N553Q, N560Q, N580Q, N612Q, N650Q, and N665Q) were constructed by site-directed mutagenesis with Q5-site directed mutagenesis kit (NEB, E0554S), and those proteins were expressed and purified in same manner for wild type of CEACAM5.

### Construction of a Fab Library and Panning

A combinatorial phage-displayed human Fab library (1×10¹¹ clones) was constructed by grafting naturally occurring V(D)J recombination regions of heavy chain (HC) and VJ recombination regions of light chain (LC) into the IGHV3-11 and IGKV1-39 germline framework, respectively. In order to amplify complementary determining region 3 (CDR3) and J genes for HC or LC, RNA extracted from PBMCs of 50 healthy blood donors was used for cDNA synthesis with Superscript™ IV first-strand Synthesis System (Invitrogen, 18091050) and random hexamer and oligo dT primers were applied to annealing step. Incorporation of amplified CDR3 and J-genes into the selected human frameworks was performed by subsequent overlapping PCR with Q5 polymerase (NEB, M0491), and prepared insert DNA and pCAT2 plasmid (in-house plasmid, modified pCom3X) were digested with NotI and ApaI restriction enzymes. Digested insert and vector were ligated in 1:1 ratio by using T4 DNA ligase (NEB, M0202). Circularized plasmid DNA was transformed into TG1 E. coli competent cells (Lucigen, 605502), and 100 times electroporation were carried out. M13KO7 helper phage (NEB, N0315S) was amplified in TOP10F′ E. coli and then used for subsequent phage-displayed library production. For panning, phages were pre-blocked with 3% bovine serum albumin (BSA) in PBS (w/v) for 1 hour at room temperature. Blocked phages incubated with 10 nM biotinylated CEACAM5 A3B3-Fc for 1 hour at room temperature in the presence of 300 nM competitors comprising CEACAM5 A1B1-Fc, CEACAM5 A2B2-Fc, and CEACAM6 AB-Fc. Bound phages were separated by streptavidin coated magnetic beads (Invitrogen, 11-205-D) and washed 10 times with 1 mL of PBS pH 7.4 containing 0.1% Tween-20 (w/v). Elution of bound phages was conducted by adding 10 mM tris-HCl pH 8.0 containing 25 mM dithiothreitol (DTT) for 10 minutes. After three rounds of panning, binding of 192 individual clones was analyzed in ELISA and then selected clones were sequenced after plasmid rescue.

Purification of scFv, Fab, and IgG

The pCAT2 plasmid containing scFv or Fab was transformed into HB2151 E. coli competent cells, and then transformed colonies were selected in ampicillin containing LB plate (100 μg/mL final concentration) for overnight in incubator at 37° C. Next day, a colony was inoculated in liquid LB+ampicillin media and cultured in 37° C. shaking incubator. 0.1 mM of isopropyl β-D-1-thiogalactopyranoside (IPTG) as final concentration was added to culture at OD₆₀₀ of between 0.4-0.6 corresponding to around 4×10⁸ cells/mL. The culture was relocated to shaking incubator set as 30° C., 200 rpm. Next day, induced E. coli cells were harvested and resuspended in 1/10 volume of periplasm extraction buffer containing polymyxin B (0.5 mg/mL in PBS pH 7.4) and then incubated on ice for an hour. Supernatant was collected then loaded into pre-packed Ni-NTA resin. Bound scFv or Fab was eluted by adding 300 mM imidazole in PBS pH 7.4 and then imidazole was removed. For IgG preparation, IgG cloned plasmid DNA was transfected to HEK239F cells, and expressed for 5-7 days post-transfection. IgGs were purified as previously described in Fc-fused antigen preparation.

### Binding Assessment in ELISA

Binding and specificity of scFv, Fab, or IgG to CEACAM related proteins were analyzed through indirect ELISA. Briefly, Fc-fused CEACAM5 polypeptides, CEACAM6 polypeptides, extra-cellular domain of CEACAM5 (R&D systems, 4128-CM), CEACAM6 (R&D systems, 3934-CM), or cynomolgus CEACAM5 (Sino biological, 90891-C08H) was coated on a 96 well plate (Corning, 3690) at 200 ng/well (50 μL volume) in PBS for 2 hours at room temperature. Blocking was carried out with 3% BSA in PBS for overnight at 4° C. Next day, various concentration of scFv, Fab, or IgG was treated to antigen coated plates and incubated for 1 hour at room temperature. After washing three times, anti-FLAG mouse antibody (M2 clone)-HRP conjugated (Sigma, A8592, 1:3000 dilution) or anti-human kappa goat antibody-HRP conjugated (Invitrogen, A18853, 1:3000 dilution) was treated for binding detection. Same volume of TMB (Thermo, PI34028) was added as a substrate, and then enzymatic reaction was stopped by adding of 2N sulfonic acid. The monomeric Fc fused-CD16a (Fc γRIIIa), CD64 (Fc γRIa) and CD32 (Fc γRIIa) were obtained from Prof. Wei Li (University of Pittsburgh, PA) and used for Fc gamma receptor (FcγR) binding test.

### Results

Isolation of Clone #1 Fab Specifically targeting A3B3 Domains of CEACAM5 with High Specificity

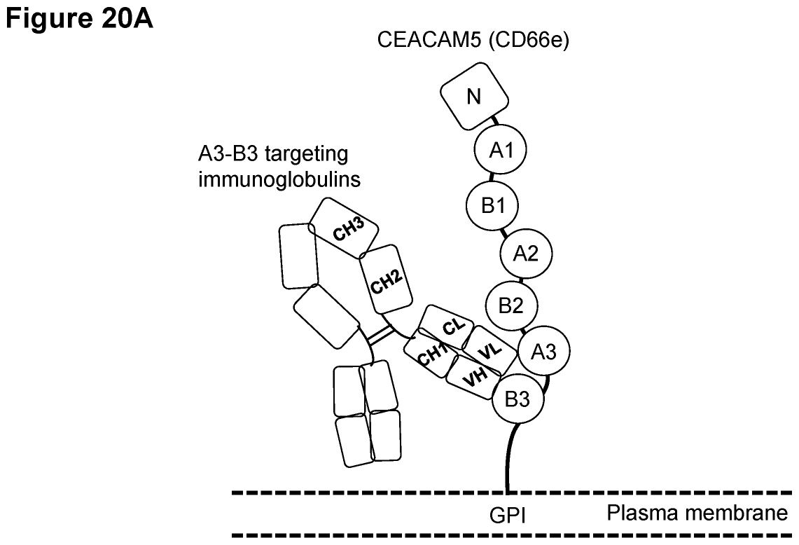

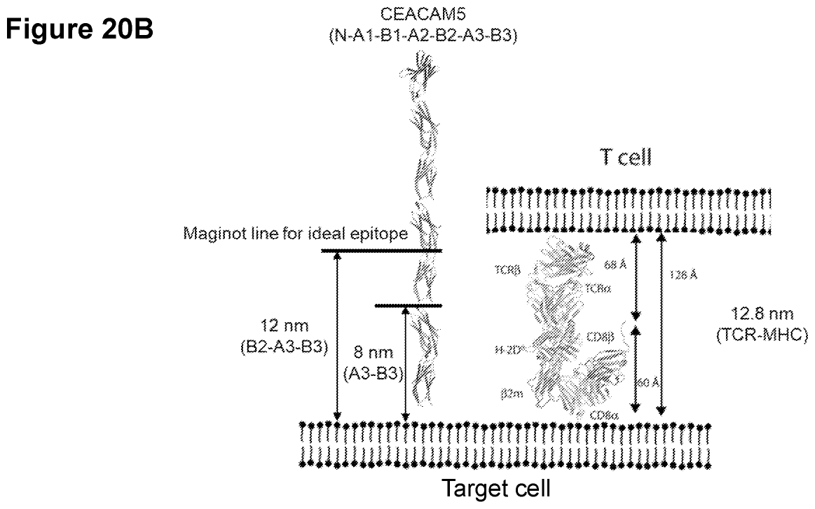

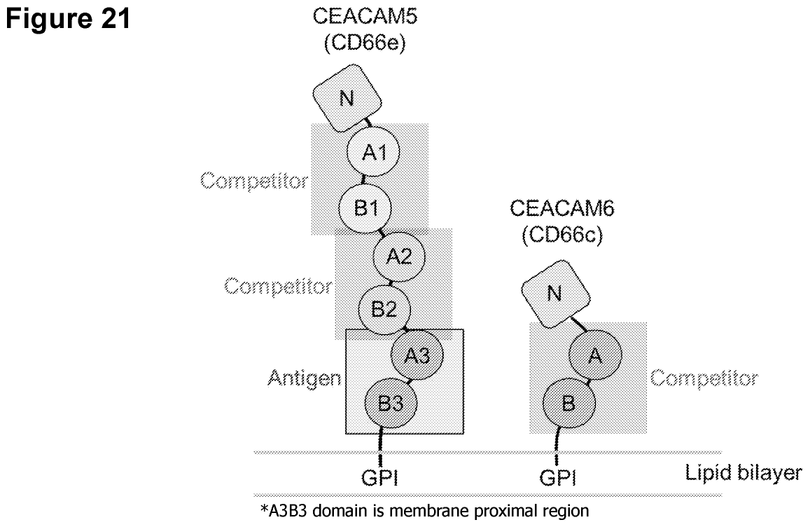

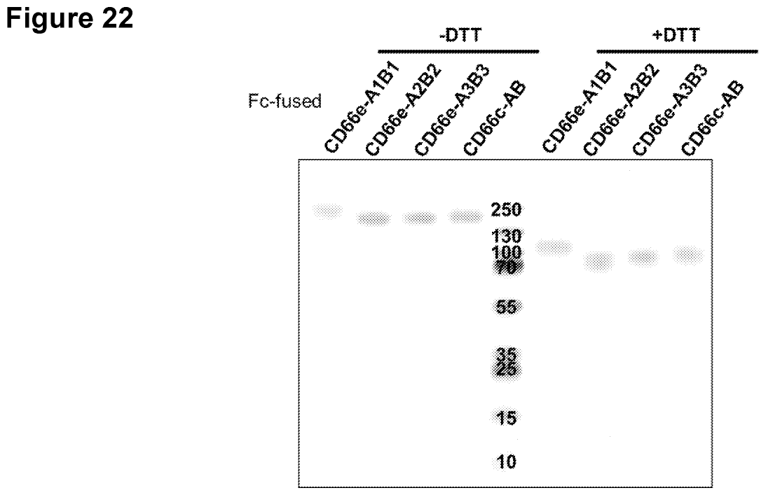

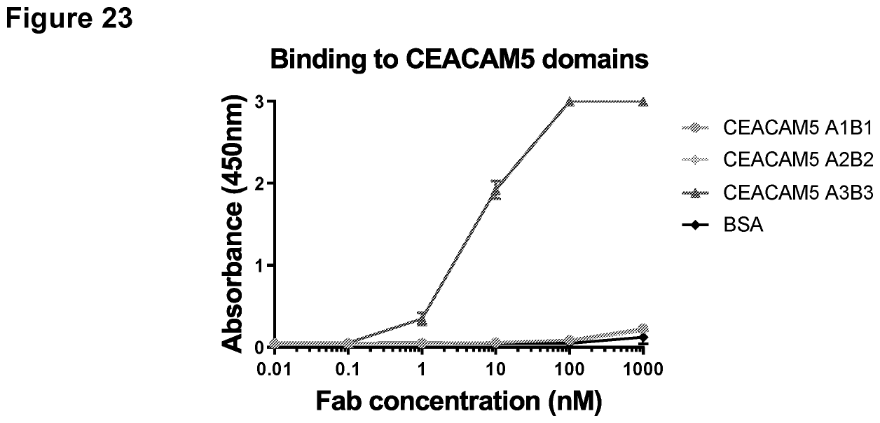

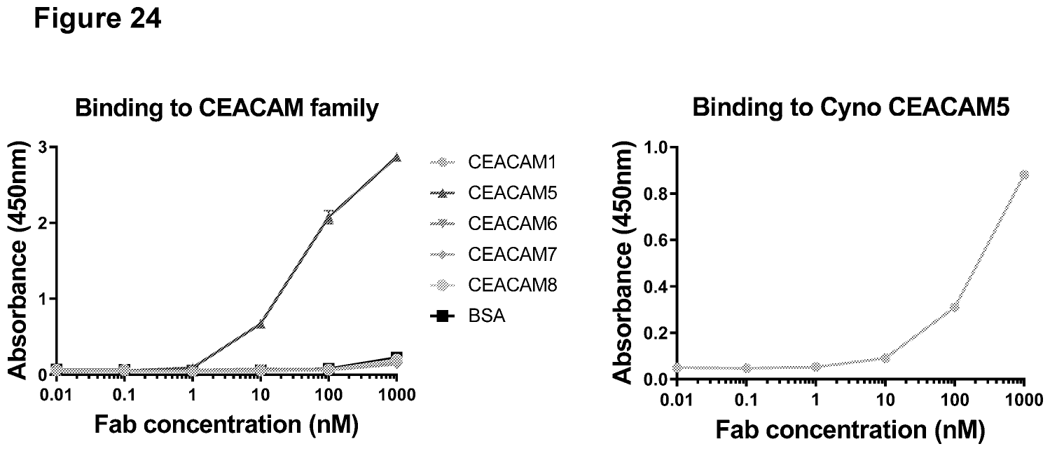

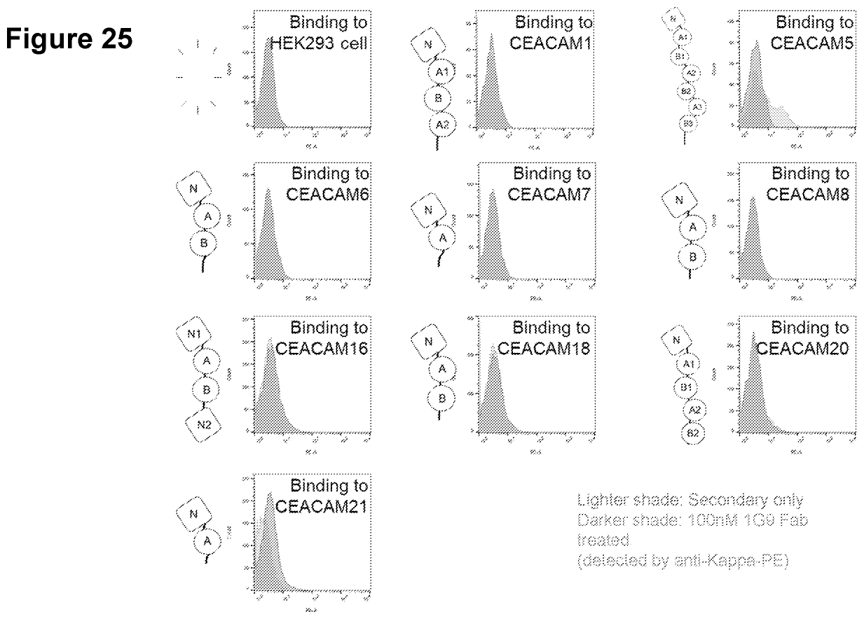

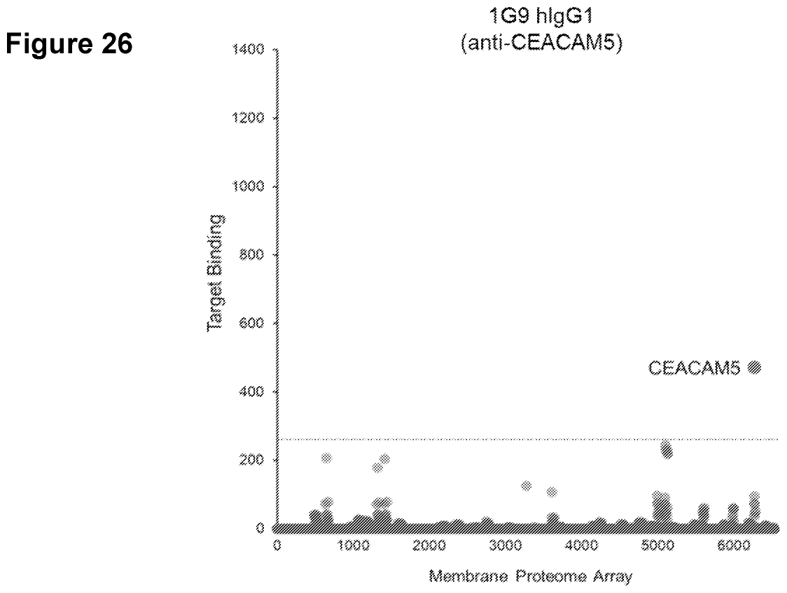

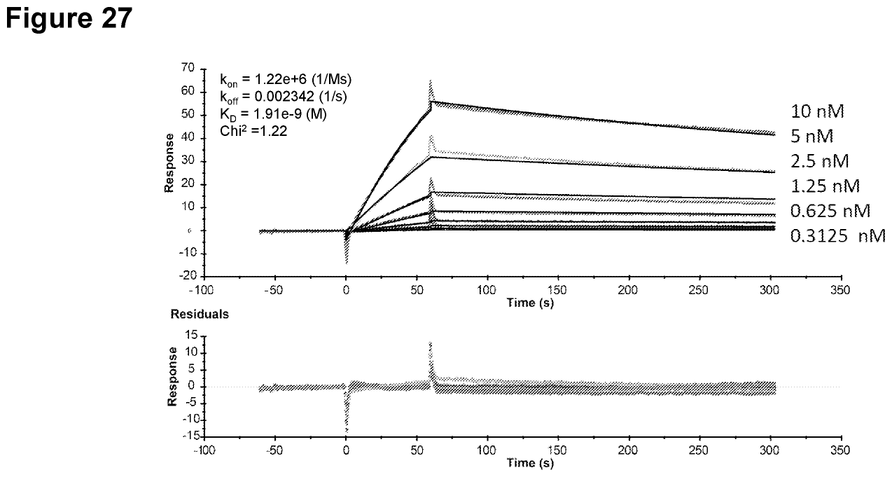

The following was performed to develop anti-CEACAM5 antibodies (e.g., fully human antibodies). Briefly, domains of CEACAM5 and CEACAM6 with fusion of human IgG1 Fc were designed and purified. A3B3 domain was regarded as an ideal epitope for targeting membrane proximal region of CEACAM5 (FIGS. 20A and 20B). In order to exclude potential nonspecific cross-reactive binders that can lead to toxicity in normal cells and to prevent binding to the membrane-distal region of the extracellular portion of CEACAM5, Fc fused-A1B2 and A2B2 domains of CEACAM5 and AB domains of CEACAM6 were used as competitors during phage library panning because those domains showed more than 70% primary sequence homology to A3B3 domains of CEACAM5 (FIG. 21). All recombinant proteins were purified in high-quality in SDS-PAGE analysis (FIG. 22). For a phage-displayed Fab library construction, a strategy was established to graft partial regions, occurring V(D)J and VJ recombination events, of VH and VL to human single germline VH3-11 and Vk1-39 framework, respectively. After 100 times electroporation, a large size of Fab library was generated showing 1011 specificities, and Clone #1 Fab (also referred to as 1G9 Fab) was successfully panned out through a competitive panning strategy using 10-fold molar excess of competitors with biotinylated Fc-fused A3B3 domains in every panning steps. Purified 1G9 Fab showed around 6 nM as half maximal effective concentration (EC₅₀) in an indirect ELISA (FIG. 23) and 3 nM as the equilibrium dissociation constant (KD) in BIAcore experiment against the A3B3 domains of CEACAM5 (FIG. 27) while no-significant signals were observed against A1B1, A2B2 domains of CEACAM5, or other CEACAM family members. Moreover, 1G9 IgG₁ bound to cynomolgus CEACAM5, suggesting that 1G9 based immunotherapeutics can be addressed for raised toxicity issues in monkey further since the cross-reactivity to both human and monkey CEACAM5 with comparable affinity is required for toxicity study in monkey (FIG. 24). In addition, 1G9 Fab exhibited superior specificity toward CEACAM5 in a flow-cytometry based analysis with HEK293F cells expressing different CEACAM family members (FIG. 25), and 1G9 hIgG1 showed a similar result in the Membrane Proteome Array (MPA), which is for testing antibody specificity using over 5,800 human cell lines that express different human membrane proteins (FIG. 26). Altogether, these results demonstrate that isolated 1G9 antibody binds to membrane-proximal domains of CEACAM5, A3B3 domains, in high affinity and high specificity without any off-target binding in three different experiments.

Epitope Mapping of IG9 Fab with N-Linked Glycan Removal Mutants of A3B3 Domains

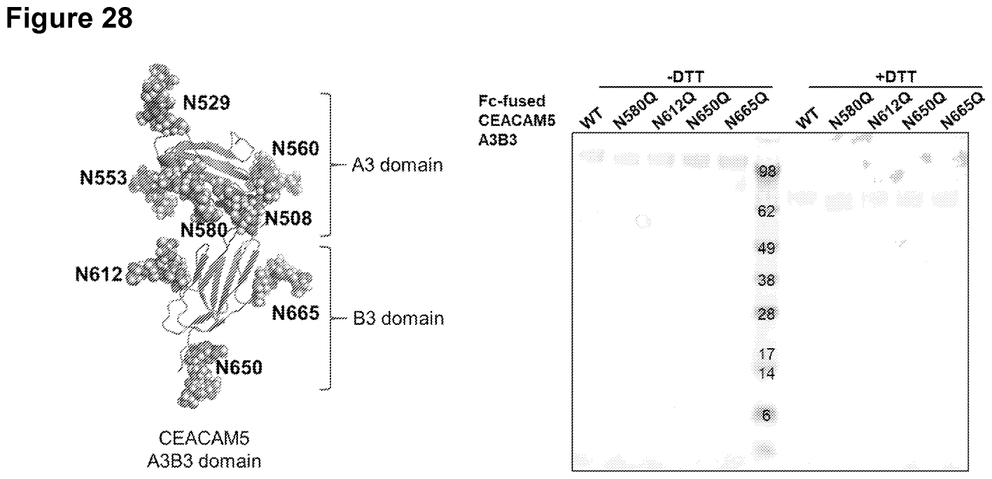

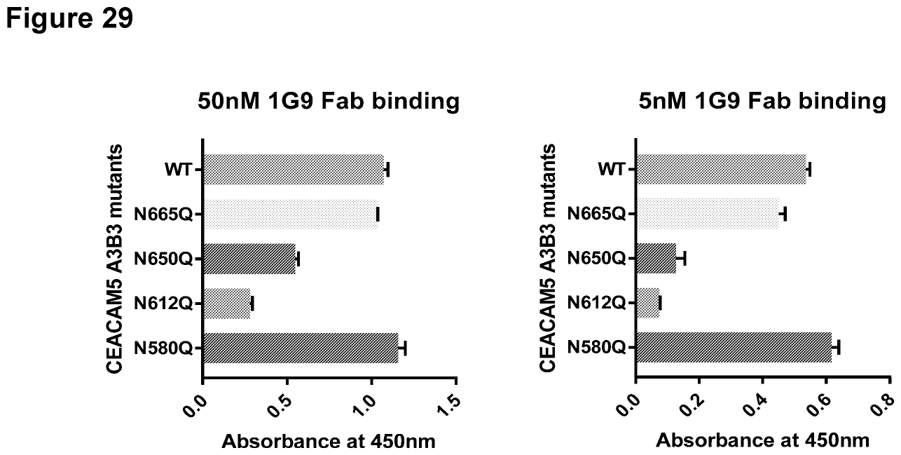

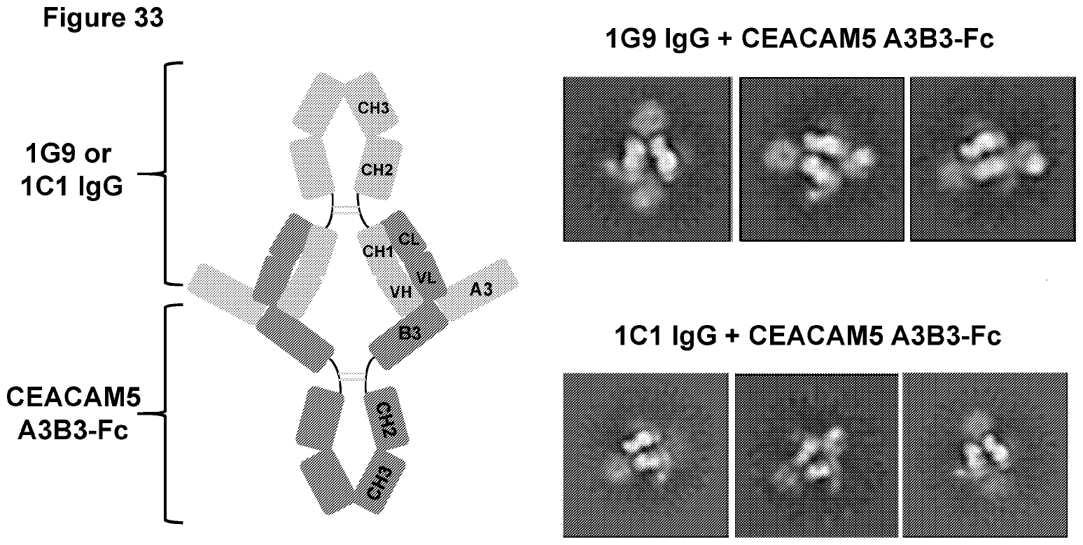

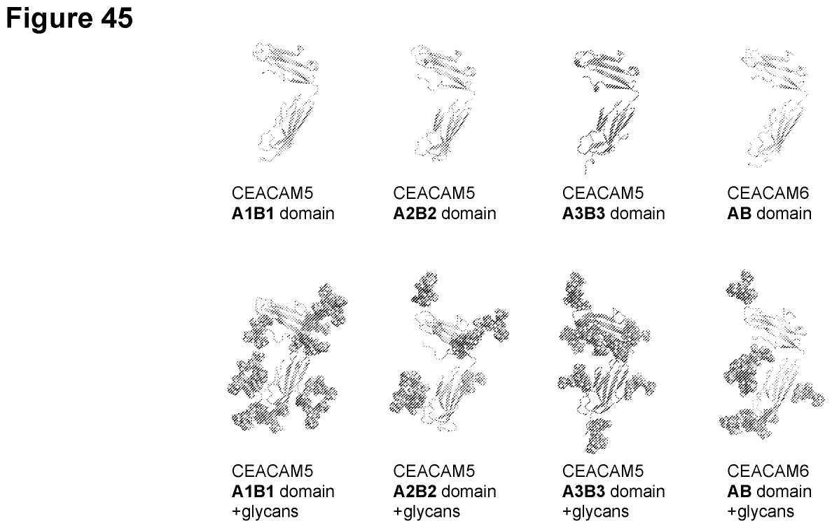

It was hypothesized that N-linked glycans in A3B3 domains of CEACAM5 may be involved in the binding of 1G9 Fab either directly or indirectly because the A3B3 domains have eight N-linked glycosylation motifs (N-X-S/T; X is natural 19 amino acids except for proline) at N508, N529, N553, N560, N580 in A3 domain and N612, N650, N665 in B3 domain. Those N-linked glycans may cover exposed surface of A3B3 domains. To explore the contribution of N-linked glycans to generating epitopes for 1G9 Fab, eight mutant proteins of A3B3 domains were constructed with substituted asparagine (N) to glutamine (Q) at each N-glycosylation motif. The N580Q, N612Q, N650Q, and N665Q mutant proteins were successfully purified with lowering yield, but other four mutants, N508Q, N529Q, N553Q, and N560Q, were not expressed in HEK293F (FIG. 28). With those mutant proteins, 1G9 Fab showed attenuated binding to N612Q and N650Q mutants of B3 domain of CEACAM5 in an indirect ELISA. On the other hand, N580Q mutation slightly improved the binding of 1G9 Fab (FIG. 29). These results were corresponded to an image obtained in the electron microscopy (EM) with the complex of 1G9 hIgG and A3B3-Fc (FIG. 33). To visualize location and orientation of N-linked glycans of CEACAM5 and CEACAM6, molecular structures were modeled with added N-linked glycans to the modeling structure. Those four domains showed very similar tertiary structure as expected because they shared over 70% primary sequences and were already known as having Ig constant region-type 2-like (IgC2-like) domains. However, addition of N-linked glycans into structure models generated largely different antigenic surfaces resulting in creation of unique conformational epitopes within each domain (FIG. 45). Taken together, these results demonstrate that 1G9 Fab binds to A3B3 domain of CEACAM5 and three N-linked glycans at M580, N612 and N650 are involved in major epitopes.

### Example 2—In Vitro Pre-Clinical Efficacy Assessment for hIgG1 and CAR-T Cell Lines

NCI-H660, Du145, and 293T cells were purchased from ATCC. Du145 was maintained EMEM supplemented with 10% v/v FBS (Gibco) and 1% penicillin-streptomycin (P/S, Gibco). 293T was maintained DMEM supplemented with 10% FBS and 1% P/S. NCI-H660 was cultured RPMI1640 (ATCC) supplemented with 5% FBS, 1× insulin-transferrin-selenium (ITS-G Giboc), 10 nM hydrocortisone, extra 2 mM L-glutamine, and 1% P/S. Du145-CEACAM5 cells, stably expressed CEACAM5, was generated by stable infection with lentiviruses derived from the pLenti lentiviral plasmid (Origene, RC206434L3) using established methods, and were cultured in EMEM supplemented with 10% FBS, 1% P/S, and 1 μg/mL puromycin (Gibco).

### Generation of CEACAM Family Expressing Cells

Freestyle 293F cells (Thermofisher) was used for generating of CEACAM family expressing cells. Plasmids cloned each gene for human CEACAM members were transfected to the cells with PEI Max (Polysciences). Each gene for human CEACAM members with FLAG-tag at C-terminal were purchased from Origene (RC224086 for CEACAM21; RC214882 for CEACAM20; RC215478 for CEACAM18; RC224965 for CEACAM16; RC230069 for CEACAM1; RC212214 for CEACAM7; RC204740 for CEACAM8; RC202454 for CEACAM6; and RC206434 for CEACAM5).

Generation of Third Generation of CAR-T Cells Using scFv Having the CDRs of Clone #1

The DNA for a third generation of CAR encoding an scFv having the CDRs of Clone #1, a human IgG₄ hinge, a CD28 transmembrane region, and costimulatory cytosolic domains of CD28, 4-1BB, and CD3 zeta was cloned into pLVX-EF1a-IRES-ZsGreen1 by EcoRI and BamHI restriction enzymes and T4 DNA ligase. For the production of lentiviral supernatant, the day before transduction, 293T cells were seeded at 4×10⁶ cells per T75 flask. After 24 hours, the lentiviruses were generated by co-transfecting 293T cells with plasmids encoding anti-CEACAM5 CAR (8 μg), pMD.2G (2 μg), and a packaging vector psPAX2 (4 μg) using PEI-based transfection system. Supernatants were collected after 48 hours and 72 hours and filtered through a 0.45 μm membrane. Then, pan T cells (Precision for medicine) were activated by Dynabeads human T-activator CD3/CD28 (Gibco), were transduced with lentiviral supernatants with 8 μg/mL polybrene (Sigma), followed by centrifugation for 45 minutes at 800×g (no acceleration and no deceleration), and then incubated at 37° C. 24 hours later, media with viruses was changed, and T cells were expanded in the T cell media (RPMI1640 supplemented with an extra 2 mM glutamax, 10% human serum, and 1% P/S) in the presence of hIL-2 (fed every 2 days, 50 IU/mL, Miltenyi Biotec).

### Flow Cytometry

To determine the cell surface expression levels of CEACAM5 or CEACAM family proteins, the cells were stained with PE-conjugated anti-CEACAM5 IgG1 (Miltenyi Biotec, 130-114-217) or PE-conjugated anti-FLAG mouse antibody (Miltenyi Biotec, 130-101-576). To confirm cell surface binding of selected antibodies, cells were treated with a hIgG1 having the CDRs of Clone #1, a hIgG1 having the CDRs of Clone #2, or the Clone #1 Fab for 1 hour at 4° C. and then stained with Alexa647-conjugated goat anti-human IgG (Invitrogen, A21445) or FITC conjugated-goat anti-human kappa light chain (Invitrogen, A18854) for 0.5 hours at 4° C. For the internalization assays, cells (1×10⁵ cells/well in 96-well plate) were treated with Alexa488-labeled antibodies for durations between 0 and 30 hours. After incubation of antibodies, cells were washed with cold PBS, and then the remained cell surface binding antibodies were analyzed using Alexa488 fluorescence. For CAR expression on anti-CEACAM5 CAR-T cells, CD4⁺ T or CD8⁺ T cells were gated using APC-conjugated anti-human CD4 antibody (ebioscience, 17-0049-42) and eFluor450-conjugated anti-human CD8 antibody (ebioscience, 48-0088-42), and CAR expression was examined using FITC-labeled recombinant Protein L (Acrobiosystems, RPL-PF141).

### In Vitro Cytotoxicity Assay

The LDH-Glo cytotoxicity assay kit (Promega, J2381) was used to measure ADCC or cell killing activity of anti-CEACAM5 CAR-T through release of cytosolic LDH from target cells. For ADCC, PBMCs or enriched NK cells from PBMCs as effector cells were incubated with the NCI-H660 or Du145 (1×10⁴ cells/well in 96-well plate) as target cells at effector-to-target (E:T) ratio of 20:1 (for PBMCs) or 5:1 (for NK cells) in the presence of a hIgG1 having the CDRs of Clone #1 or a hIgG1 having the CDRs of Clone #2 for 4 hours. For cell killing activity of anti-CEACAM5 CAR-T, control T or CAR-T cells as effector cells were incubated with NCI-H660, Du145-CEACAM5, or Du145 cells as target cells at the indicated E:T ratio for 24 hours in 96-well plate. Controls conducted for the calculation of percent cytotoxicity (% cytotoxicity) were performed according to the manufacturer's instructions. % cytotoxicity with the following formula: (experimental-effector minimum-target minimum)/(target maximum-target minimum)×100.

The CellTiter-Glo Luminescent cell viability assay kit (Promega, G7571) was used to measure cell viability in the presence of the hIgG1 having the CDRs of Clone #1. Cells (5×10³ cells/well in white 96-well plate) were plated and cultured for 12 hours prior to treatment with the indicated antibodies for 72 hours. Normalized % ATP values were calculated by normalizing luminescence values for vehicle-treated wells.

### Transwell Assay

Cell migration was measured using transwell (8-μm pore size, Corning 3422). Cells (1×10⁵ cells/well) were resuspended in serum-free medium and then added to the upper chamber in the presence of the hIgG1 having the CDRs of Clone #1. Lower chambers contained 10% FBS. No FBS condition was conducted as negative control. After incubation for 3 days at 37° C., cells remained at the upper surface of the membrane were removed using a swab, while the cells that migrated to the lower membrane surface were fixed and stained with 0.5% crystal violet in 20% methanol solution. Cells migrating through the filter were quantified by dissolving the stained cells using 10% acetic acid solution and then absorbance was measured at 590 nm. Normalized % of migration values were calculated by normalizing absorbance values for 10% FBS-treated wells in absence of antibody.

### Cytokine ELISA

Control T cells and CAR-T cells were plated in 24-well plate (2×10⁴ cells/well) and co-cultured with target cells at the indicated E:T ratio for 48 hours at 37° C. Subsequently, cytokine levels of supernatants were analyzed using human IFNγ, TNFα, IL-2, IL-4, IL-13, and GM-CSF ELISA kit (Thermofisher) respectively, following the manufacturer's instructions.

### Results

IG9 hIgG1 Induces CEACAM5-Dependent ADCC

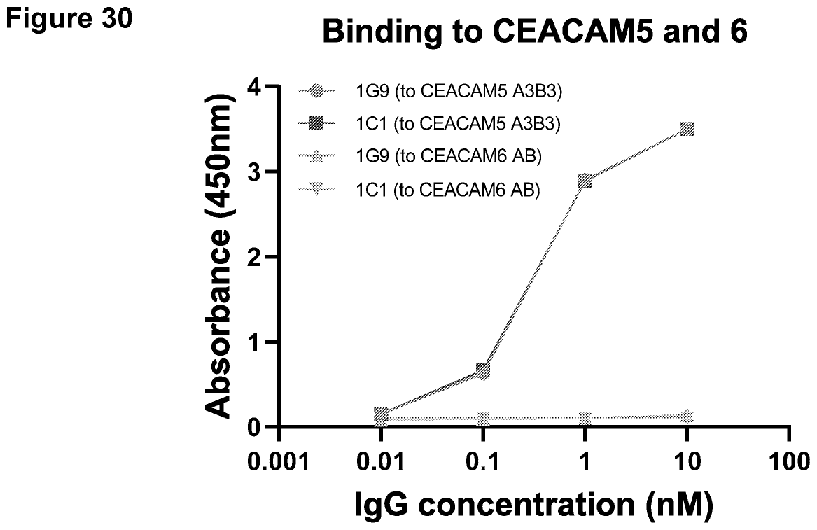

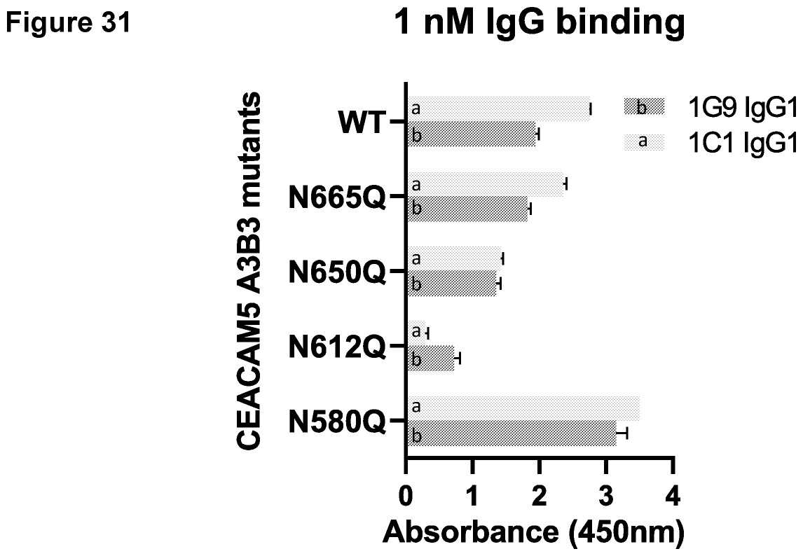

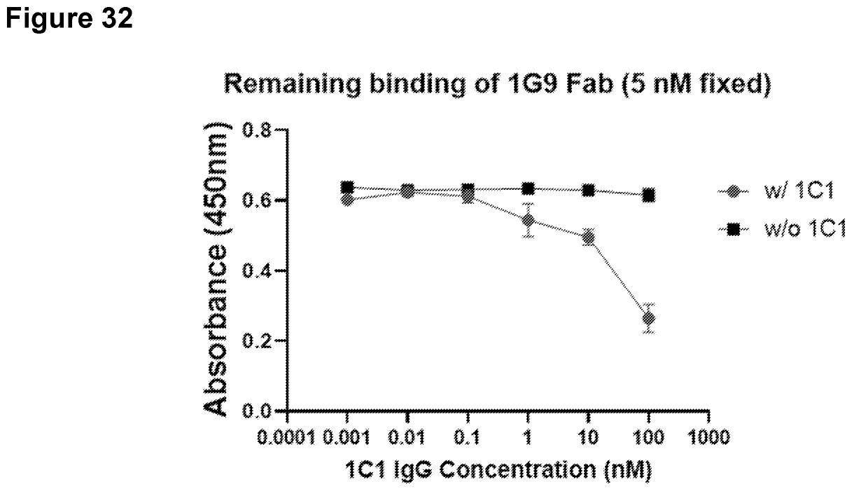

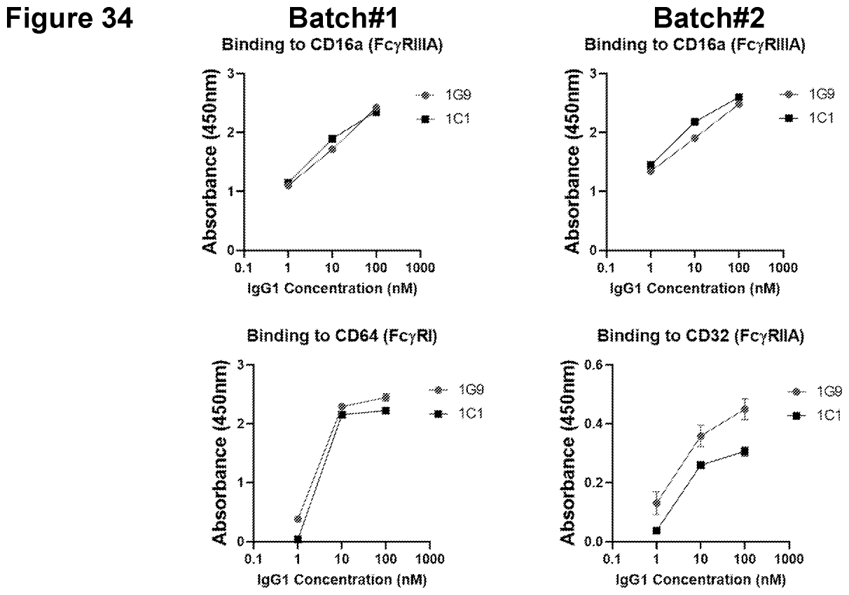

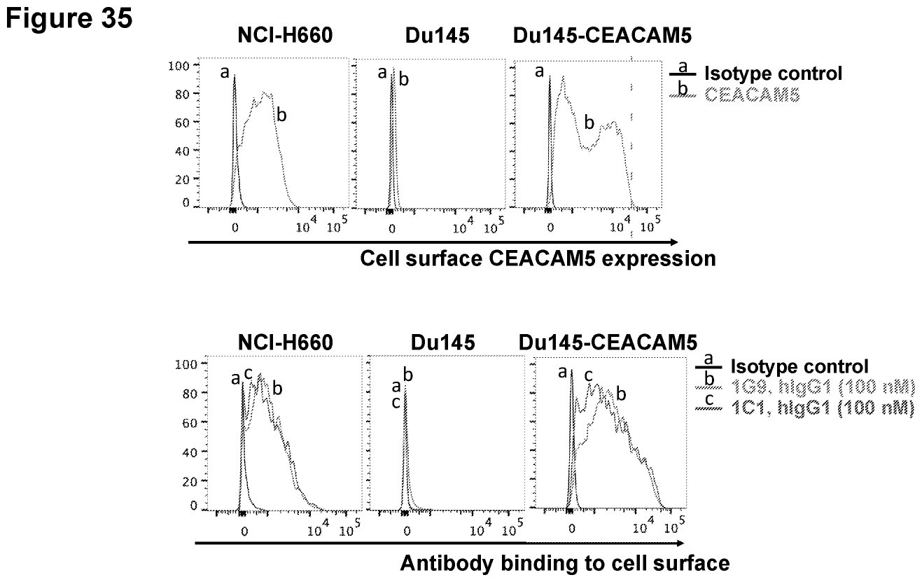

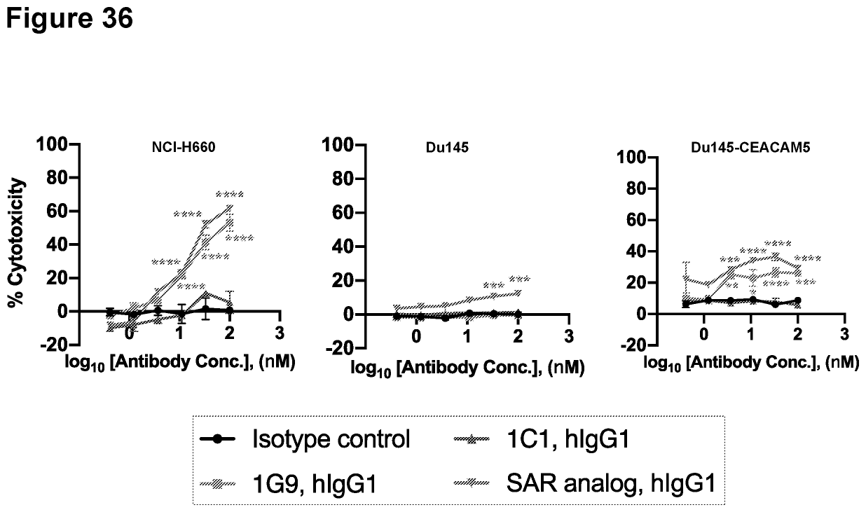

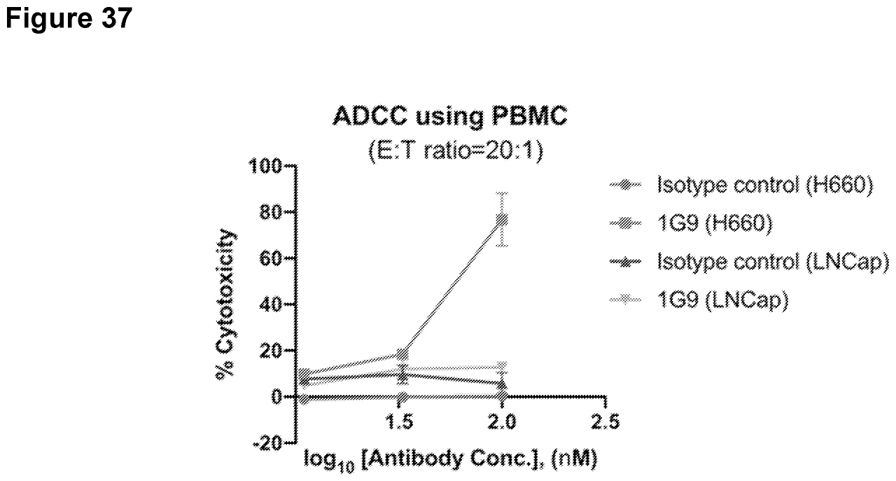

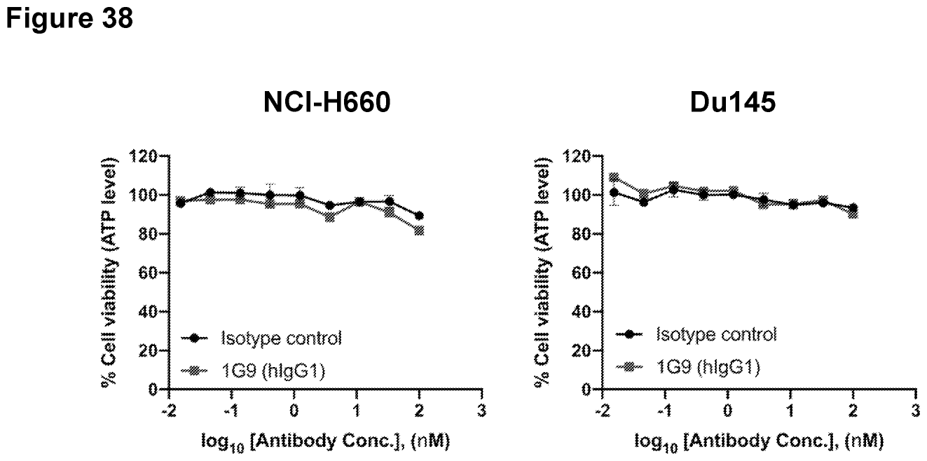

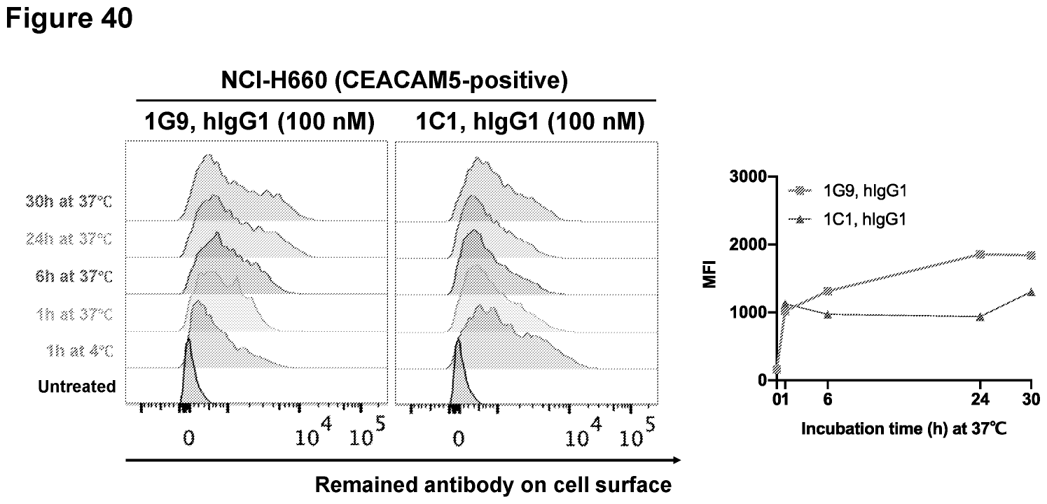

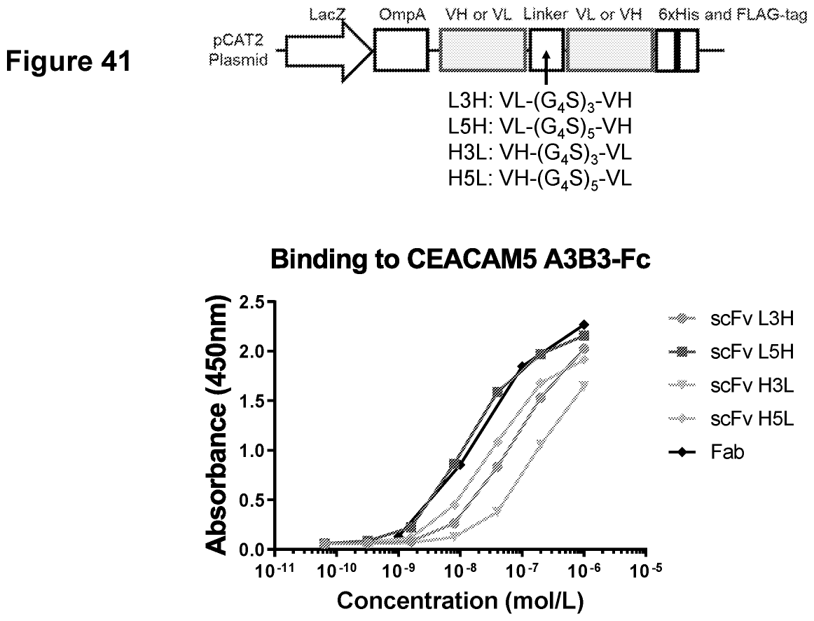

During the characterization of 1G9, Clone #2 Fab (also referred to as 1C1 Fab) also was isolated. The 1C1 hIgG1 specifically bound to A3B3 domains of CEACAM5 like 1G9 hIgG1 (FIG. 30) and also showed similar binding to mutants of A3B3 domains, suggesting that N-linked glycans at N580, N612, and N650 were also involved in epitope for 1C1 (FIG. 31). In competitive ELISA, those two antibodies competed for binding to CEACAM5, suggesting that two antibodies bind overlapping epitope in A3B3 domains (FIG. 32). The binding of 1G9 and 1C1 hIgG1 to CEACAM5-positive NCI-H660 and CEACAM5-negative Du145 cells was assessed then. Both 1G9 and 1C1 hIgG1 showed specific binding to NCI-H660 cells. Notably, Du145-CEACAM5 cells, engineered to express CEACAM5, displayed binding of 1G9 and 1C1 hIgG1 on the cell surface distinct from CEACAM5-negative Du145 cells (FIG. 35). Next, the ADCC activity of 1G9 and 1C1 hIgG1 were tested against CEACAM5-positive target cells using an LDH release cytotoxicity assay. Primary NK cells enriched from human peripheral blood mononuclear cells (PBMCs) as effector cells and NCI-H660 or Du145 cells as target cells were used. 1G9 hIgG1 caused a dose-dependent ADCC activity against CEACAM5-positive NCI-H660 cells and CEACAM5-positive Du145 cells, but not CEACAM5-negative Du145 cells (FIG. 36) and LNCap (FIG. 37). Interestingly, no ADCC activity was observed in treatment of 1C1 hIgG1, although 1C1 hIgG1 showed a similar binding affinity to A3B3 domains of CEACAM5 and cells as well as Fc gamma receptor (FcγR) (FIG. 34). To examine why 1C1 hIgG1 showed a different ADCC activity from 1G9, electro microscopic (EM) images were examined to elucidate minor differences such as binding orientation or binding site, but 1C1 hIgG1 bound to A3B3 domains similar to 1G9 hIgG1 and both antibodies dominantly bound to B3 domain of CEACAM5 in EM image. Second, cell killing activity of 1G9 or 1C1 hIgG1 was tested in absence of immune cells but no cytotoxic activity against NCI-H660 cells was found (FIG. 38) by both antibodies. This result indicated that cell killing activity of 1G9 hIgG1 was clearly dependent on recruiting of FcγR-expressing immune cells including NK cells but targeting CEACAM had no effects on cell viability in absence of immune cells. Third, the rate of internalization for 1G9 and 1C1 hIgG1 was evaluated by measuring the remained antibody on the NCI-H660 cell surface after antibody treatment at 37° C. in time-course by flow cytometry (FIG. 40). Both hIgG1s did not undergo internalization up to 30 hours, suggesting that the difference in ADCC activity for both hIgG1s was unlikely to be caused by different internalization kinetics.

1G9 hIgG1 Inhibits Cell Migration and Invasion

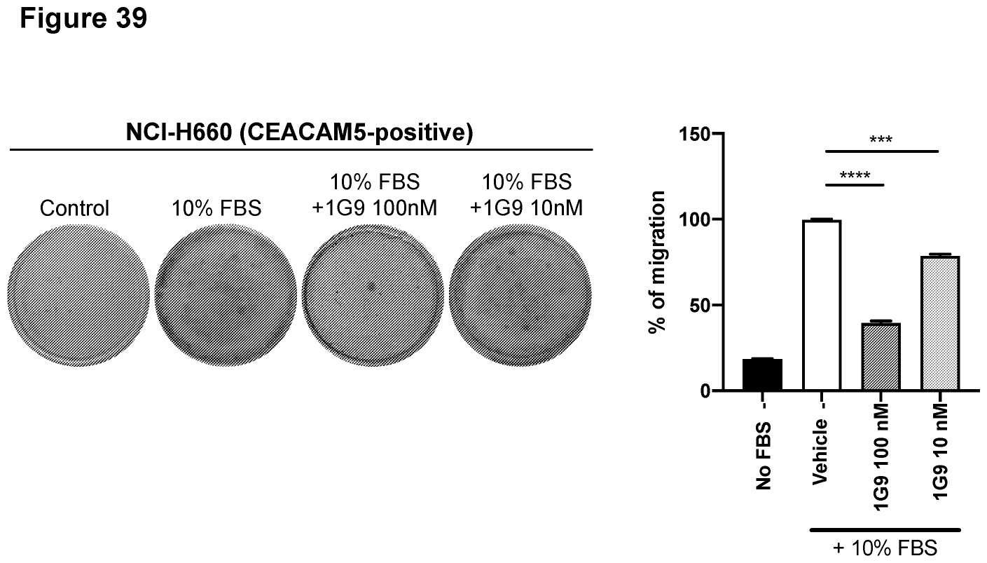

As CEACAM5 play a role in regulation of cell adhesion and migration as cell adhesion molecule, the role of 1G9 hIgG1 in prostate cancer cell migration was explored using transwell assay. The NCI-H660 and Du145-CEACAM5 cells exhibited impaired migration by treatment of 1G9 hIgG1, in a dose-dependent manner, compared with serum-treated cells while no migration effects of 1G9 hIgG1 were observed in CEACAM5-negative Du145 cells (FIG. 39). Similarly, analog of Sanofi's antibody targeting CEACAM5 (SAR408701) inhibited cell migration. These results suggested that targeting CEACAM5 by 1G9 hIgG1 can impede CEACAM5-mediated cell migration.

### Anti-CEACAM5 CAR-T Shows Strong Cytotoxicity Against CEACAM5-Positive Prostate Cancer Cells

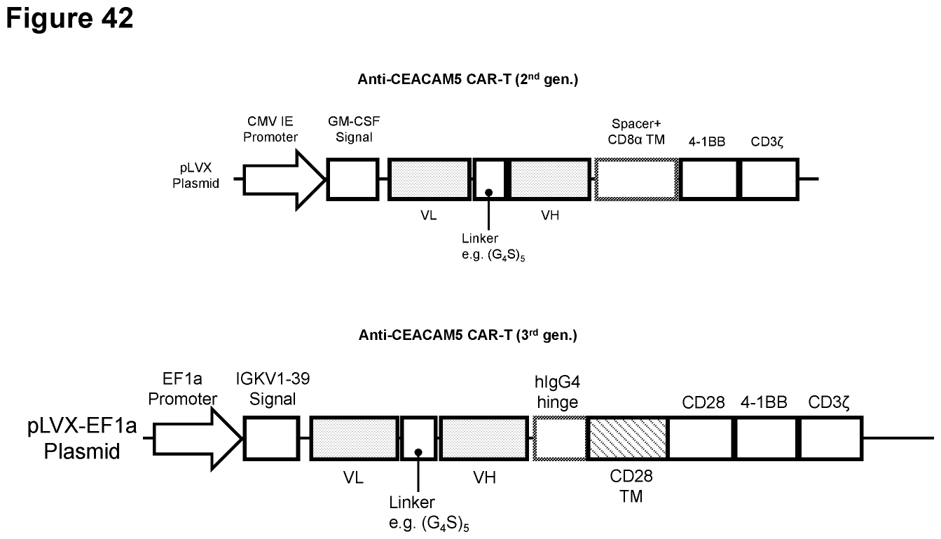

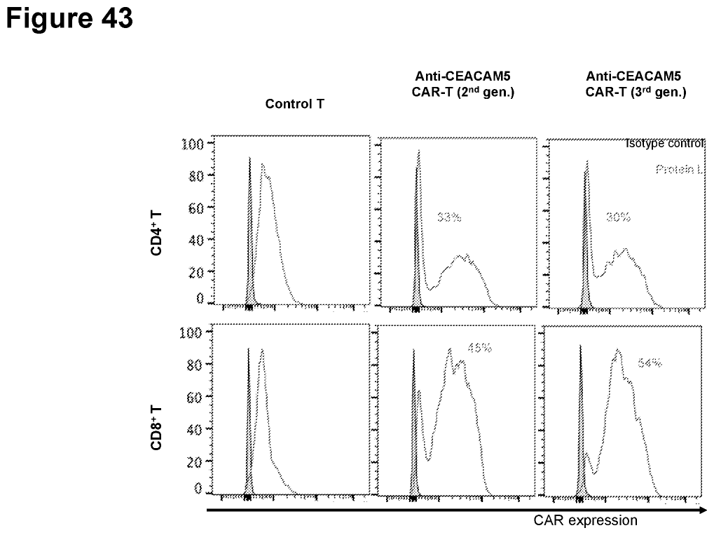

Based on the epitope mapping and limited internalization of hIgG1 1G9, it was concluded that the 1G9 antibody could be used for CAR-T cell therapy. Lentiviral 2^{nd} and 3^{rd} generation anti-CEACAM5 CAR constructs encoding a single chain variable fragment (scFv) derived from 1G9, a transmembrane domain, and a costimulatory intracellular domain as shown in FIG. 42 were generated. T cells were transduced with the 2^{nd} and 3^{rd} generation CAR constructs, and CAR expression was confirmed by flow cytometry. The expression level of 3^{rd} generation CAR in transduced T cells was 84%, including 30% in CD4⁺ T cells and 54% in CD8⁺ T cells, which was similar to the expression level of 2^{nd} generation CAR, which totaled at 78% (FIG. 43).

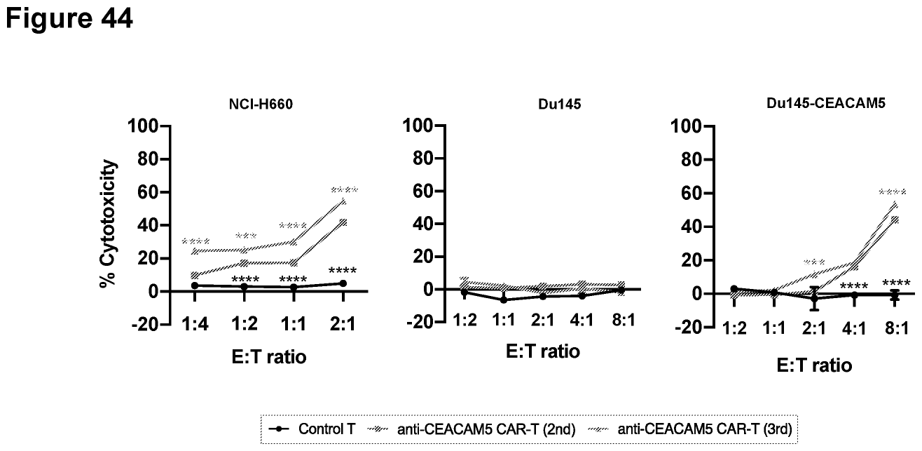

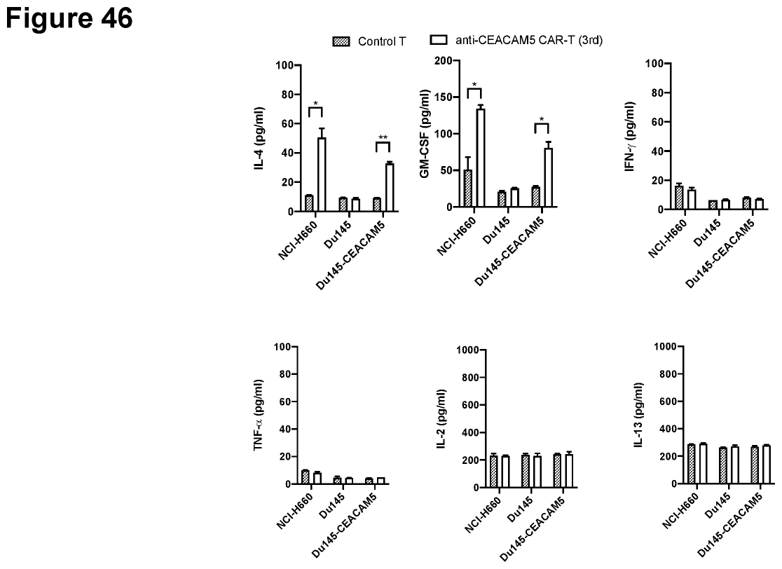

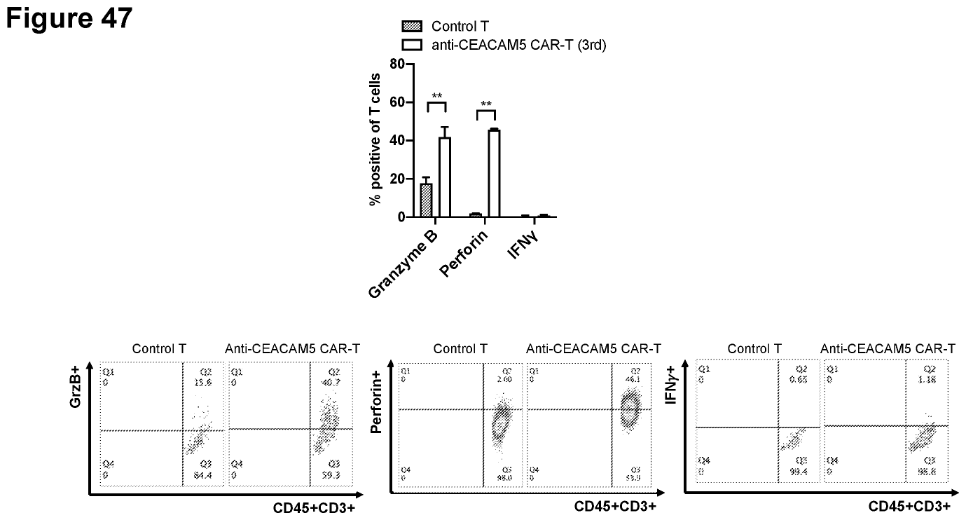

To determine the cytotoxicity of anti-CEACAM5 CAR-T cells for target cells in vitro, anti-CEACAM5 CAR-T cells were co-cultured with CEACAM5-positive NCI-H660 and Du145-CEACAM5, at the E:T ratios of 1:4, 1:2, 1:1, 2:1, 4:1 and 8:1 for 24 hours. The cytotoxicity (%) of 2^{nd} and 3^{rd} generation CAR-T cells against NCI-H660 cells was higher than that of the control T cell group at every E:T ratio. The 3^{rd} generation CAR-T cells exhibited more potent cytotoxicity compared to the 2^{nd} generation CAR-T cells (FIG. 44). Cytotoxicity of the 3^{rd} generation CAR-T cell group against Du145-CEACAM5 cells was observed at E:T ratios of 8:1, 4:1, and 2:1, but not against Du145 cells (FIG. 44). To further investigate the cytotoxic mechanism of anti-CEACAM5 CAR-T cells, the production of IFN-gamma (IFNγ), TNFα, IL-2, IL-4, IL-13, and GM-CSF from the supernatant of CAR-T after co-culture with target cells for 48 hours was analyzed. Secretion of IL-4 and GM-CSF was significantly enhanced by treatment of 3^{rd} generation anti-CEACAM5 CAR-T cells in CEACAM5-expressing NCI-H660 and Du145-CEACAM5 cells, but not CEACAM5-negative Du145 cells (FIG. 46). Additionally, analysis of the supernatant at 48 hours for granzyme B (GrzB), perforin, and IFNγ intracellular staining revealed enhanced GrzB and perforin release from 3^{rd} generation anti-CEACAM5 CAR-T cells in CEACAM5-positive NCI-H660 cell cultures (FIG. 47). The 3^{rd} generation anti-CEACAM5 CAR-T cells resulted in remarkable cytotoxicity in CEACAM5-positive NEPC cells via IL-4, GM-CSF cytokine secretion and GrzB/perforin release, likely due to the membrane proximity of 1G9 binding to the A3B3 domain, in combination with a well-designed CAR spacer.

### Example 3—In Vivo Pre-Clinical Efficacy Assessment for hIgG1 and CAR-T In Vivo Study

For evaluation of hIgG1 1G9, Du145 and Du145-CEACAM5 cells (1×10⁷ cell/mice) in 200 μL of a 1:1 mixture of PBS/Matrigel (BD Biosciences, 354234) were injected subcutaneously (s.c.) into the right flank of Balb/c scid mice (6-8 weeks old, male, The Jackson Laboratory). When the tumor volume reached approximately 200 mm³, mice were randomized, and hIgG1 1G9 (20 mg/kg or 5 mg/kg) or vehicle (buffer control) were intraperitoneally administered twice per week. For validation of CAR-T, NOD-scid IL2Rg^{null} mice (NSG mice, 6-8 weeks old, male, The Jackson Laboratory) were engrafted s.c. (right flank) with Du145 and Du145-CEACAM5 (1×10⁷ cells/mice) in 200 μL of a 1:1 mixture of PBS/Matrigel. When the tumors were 150 mm³, mice were treated with control T cells or CAR-T cells (5×10⁶ cells/mice) every 4 days, two times, via tail vein. Tumor dimensions were measured with caliper, and tumor volume was calculated by the formula V=1/2×length×(width)². Animals were euthanized when the tumor volume reached >1.0 cm³.

### Results

Anti-CEACAM5 hIgG1 IG9 and CAR-T Cells Inhibited Growth of CEACAM5-Positive Prostate Cancer Xenografts

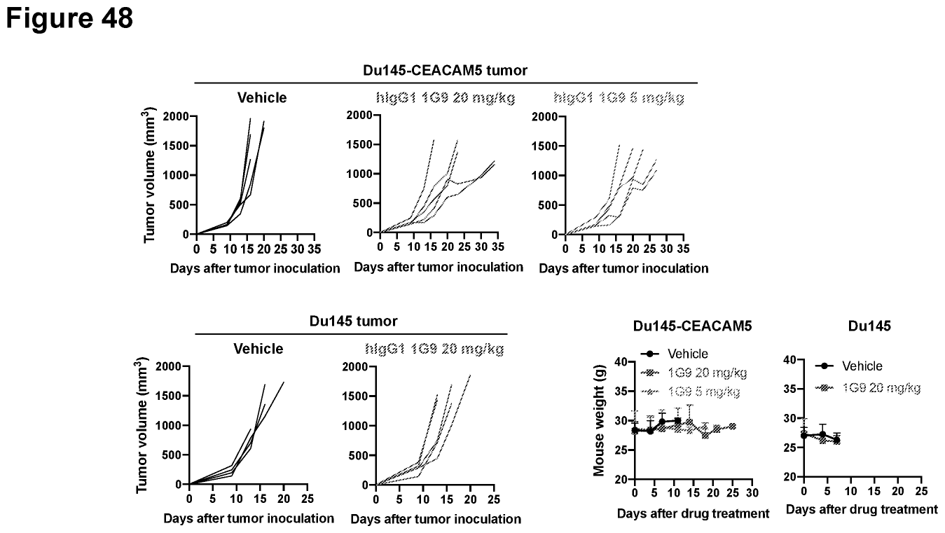

To evaluate the cytotoxic potential of hIgG1 1G9 in vivo, mouse xenograft models of Du145 and Du145-CEACAM5 were used. Mice bearing approximately 200 mm³ tumors were treated with hIgG1 1G9 at 20 or 5 mg/kg. hIgG1 1G9 significantly slowed the growth of Du145-CEACAM5 tumor (FIG. 48) and improved mouse survival compared to those of vehicle-treated mice. For CEACAM5-negative Du145 tumors, no significant difference was observed in tumor growth and mouse survival between vehicle and hIgG1 1G9. The body weight of mice, monitored as an indicator of drugs toxicity, was similar in the treatment and vehicle groups.

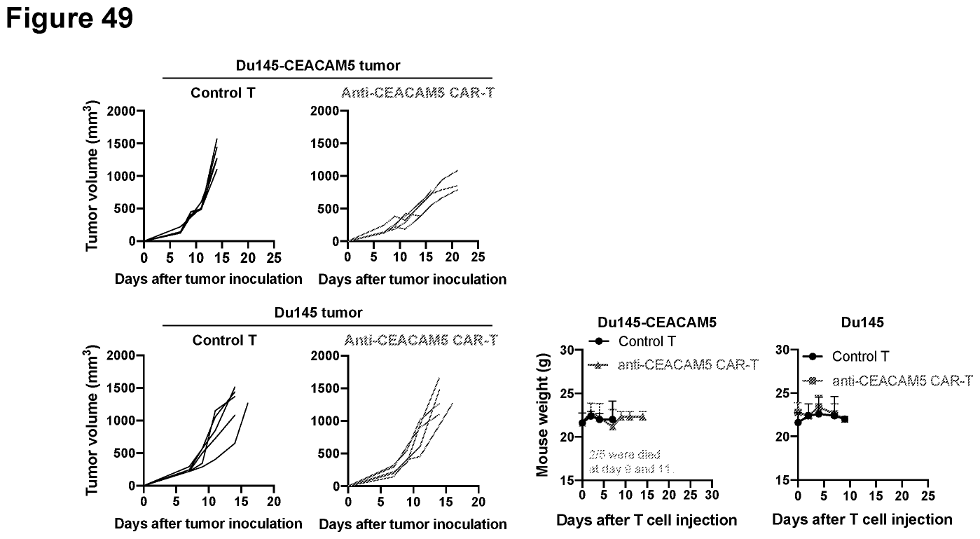

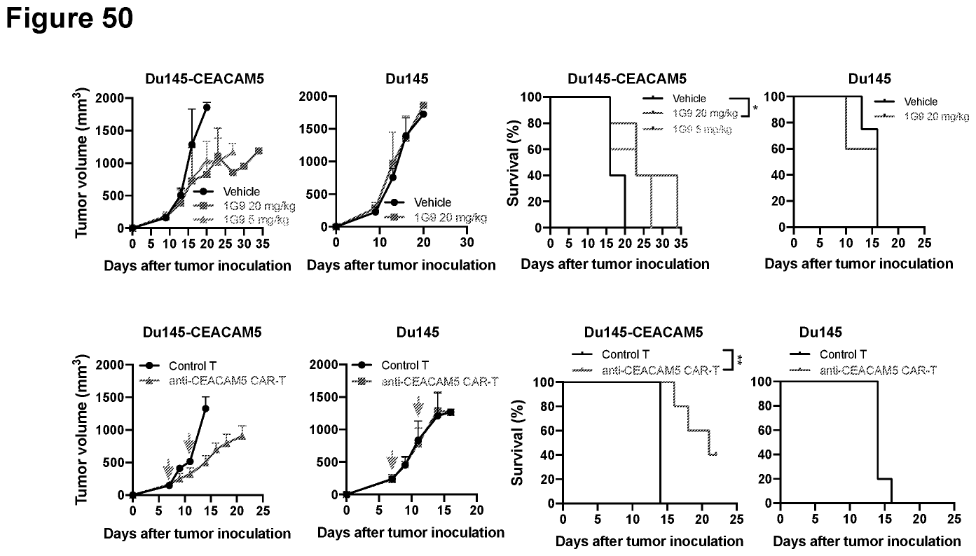

The cytotoxicity of anti-CEACAM5 CAR-T cells also was investigated in mice using xenograft models. NSG mice were s.c. implanted with Du145 and Du145-CEACAM5 cells, and control T cells or anti-CEACAM5 CAR-T cells were intravenously injected into mice via tail vein two times. Anti-CEACAM5 CAR-T cell-treated mice suppressed tumor growth and had longer survival compared to control T cell-treated mice in Du145-CEACAM5 tumor, but not Du145 tumor (FIG. 49). During CAR-T cell treatment, 2 of 5 mice showed 10% body weight loss for Du145-CEACAM5 tumors, but not Du145 tumors, and they died at day 9 and 11 after CAR-T injection. This result suggests that the anti-CEACAM5 CAR-T cells exhibited target-mediated systemic toxicity in association with potent anti-tumor effects in some mice. Taken together, these results demonstrate that anti-CEACAM5 hIgG1 1G9 and CAR-T cells exhibit in vivo anti-tumor efficacy depending on CEACAM5 expression, consistent with in vitro results (FIG. 50).

### Example 4—Designing BiKE's from Binders Having the Ability to Bind to a Human CD66e Polypeptide

The heavy and light variable domains of the Fab's of Clones #1 and #2 along with an anti-NKG2A are used to create vectors designed to express BiKEs (BiKE #1 and BiKE #2) having the ability to bind to CD66e polypeptides and NKG2A polypeptides.

### Preparation of Anti-CEA×NKG2A BiKE

A synthesized gene for digested gene for anti-CEA×NKG2A BiKE is digested by NotI and NheI restriction enzymes and then is ligated into pCAT2 vector by T4 DNA ligase. Re-circularized plasmid DNA is transformed into HB2151 E. coli competent cells, and then transformants are selected in ampicillin containing LB plate (100 μg/mL final concentration) for overnight in incubator at 37° C. Next day, a colony is inoculated in liquid LB+ampicillin media and is cultured in 37° C. shaking incubator until mid-log growth phase. At OD₆₀₀ of between 0.4˜0.6, 0.1 mM of isopropyl 3-D-1-thiogalactopyranoside (IPTG) is added to the culture, and it is continued to grow on shaking incubator at 30° C. and 200 rpm. Next day, induced E. coli cells are harvested by centrifugation at 6,000 g for 10 minutes. The cell pellet is resuspended in 1/10 volume of periplasm extraction buffer containing polymyxin B (0.5 mg/mL in PBS pH 7.4) and is then incubated on ice for an hour. The supernatant is collected by centrifugation at 12,000 g for 20 minutes. Filtered supernatant is loaded into a disposable column, which is pre-packed with Ni-NTA resin. After loading, the resin is washed with 5-bed volume of 30 mM imidazole in PBS pH 7.4, then Bound BiKE molecule is eluted by adding 300 mM imidazole in PBS pH 7.4. The remaining imidazole is removed through PD-10 desalting column, pre-packed with PBS pH 7.4.

### Example 5—Other Embodiments

Embodiment 1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).
   Embodiment 2. The antibody of embodiment 1, wherein said antibody comprises the ability to bind to SEQ ID NO:150 or SEQ ID NO:151.
   Embodiment 3. The antibody of any one of embodiments 1-2, wherein said antibody comprises said heavy chain variable domain or region of said (i).
   Embodiment 4. The antibody of embodiment 3, wherein said heavy chain variable domain or region comprises an amino acid sequence having at least 90 percent identity to the amino acid sequence set forth in SEQ ID NO:8.
   Embodiment 5. The antibody of any one of embodiments 1-2, wherein said antibody comprises said light chain variable domain or region of said (i).
   Embodiment 6. The antibody of embodiment 5, wherein said light chain variable domain or region comprises an amino acid sequence having at least 90 percent identity to the amino acid sequence set forth in SEQ ID NO:16.
   Embodiment 7. The antibody of any one of embodiments 1-2, wherein said antibody comprises said heavy chain variable domain or region of said (ii).
   Embodiment 8. The antibody of embodiment 7, wherein said heavy chain variable domain or region comprises an amino acid sequence having at least 90 percent identity to the amino acid sequence set forth in SEQ ID NO:24.
   Embodiment 9. The antibody of any one of embodiments 1-2, wherein said antibody comprises said light chain variable domain or region of said (ii).
   Embodiment 10. The antibody of embodiment 9, wherein said light chain variable domain or region comprises an amino acid sequence having at least 90 percent identity to the amino acid sequence set forth in SEQ ID NO:32.
   Embodiment 11. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).
   Embodiment 12. The antigen binding fragment of embodiment 11, wherein said antigen binding fragment comprises the ability to bind to SEQ ID NO:150 or SEQ ID NO:151.
   Embodiment 13. The antigen binding fragment of any one of embodiments 11-12, wherein said antigen binding fragment comprises said heavy chain variable domain or region of said (i).
   Embodiment 14. The antigen binding fragment of embodiment 13, wherein said heavy chain variable domain or region comprises an amino acid sequence having at least 90 percent identity to the amino acid sequence set forth in SEQ ID NO:8.
   Embodiment 15. The antigen binding fragment of any one of embodiments 11-12, wherein said antigen binding fragment comprises said light chain variable domain or region of said (i).
   Embodiment 16. The antigen binding fragment of embodiment 15, wherein said light chain variable domain or region comprises an amino acid sequence having at least 90 percent identity to the amino acid sequence set forth in SEQ ID NO:16.
   Embodiment 17. The antigen binding fragment of any one of embodiments 11-12, wherein said antigen binding fragment comprises said heavy chain variable domain or region of said (ii).
   Embodiment 18. The antigen binding fragment of embodiment 17, wherein said heavy chain variable domain or region comprises an amino acid sequence having at least 90 percent identity to the amino acid sequence set forth in SEQ ID NO:24.
   Embodiment 19. The antigen binding fragment of any one of embodiments 11-12, wherein said antigen binding fragment comprises said light chain variable domain or region of said (ii).
   Embodiment 20. The antigen binding fragment of embodiment 19, wherein said light chain variable domain or region comprises an amino acid sequence having at least 90 percent identity to the amino acid sequence set forth in SEQ ID NO:32.
   Embodiment 21. The antibody of any one of embodiments 1-10, wherein said antibody is a monoclonal antibody.
   Embodiment 22. The antibody of any one of embodiments 1-10 and 21, wherein said antibody is an scFv antibody.
   Embodiment 23. The antigen binding fragment of any one of embodiments 11-20, wherein said antigen binding fragment is monoclonal.
   Embodiment 24. The antigen binding fragment of any one of embodiments 11-20 and 23, wherein said antigen binding fragment is an Fab.
   Embodiment 25. A chimeric antigen receptor comprising an antigen binding domain, a hinge, a transmembrane domain, and one or more signaling domains, wherein said antigen binding domain comprises an antibody or an antigen-binding fragment of any one of embodiments 1-24.
   Embodiment 26. The chimeric antigen receptor of embodiment 25, wherein said antigen binding domain comprises a scFv having the ability to bind to a CD66e polypeptide.
   Embodiment 27. The chimeric antigen receptor of any one of embodiments 25-26, wherein said hinge comprises a hinge set forth in FIG. 13.
   Embodiment 28. The chimeric antigen receptor of any one of embodiments 25-27, wherein said transmembrane domain comprises a transmembrane domain set forth in FIG. 14.
   Embodiment 29. The chimeric antigen receptor of any one of embodiments 25-28, wherein said chimeric antigen receptor comprises one or more signaling domains set forth in FIG. 15.
   Embodiment 30. A cell comprising a chimeric antigen receptor of any one of embodiments 25-29.
   Embodiment 31. The cell of embodiment 30, wherein said cell is a T cell, a stem cell, or an NK cell.
   Embodiment 32. A cell engager comprising a first antigen binding domain, a linker, and a second antigen binding domain, wherein said first antigen binding domain comprises an antibody or an antigen-binding fragment of any one of embodiments 1-24.
   Embodiment 33. The cell engager of embodiment 32, wherein said first antigen binding domain comprises a scFv having the ability to bind to a CD66e polypeptide.
   Embodiment 34. The cell engager of embodiment 32, wherein said first antigen binding domain is an IgG having the ability to bind to a CD66e polypeptide.
   Embodiment 35. The cell engager of any one of embodiments 32-34, wherein said linker comprises a linker set forth in FIG. 10 or FIG. 13.
   Embodiment 36. The cell engager of any one of embodiments 32-35, wherein said second antigen binding domain binds to a polypeptide expressed on the surface of T cells.
   Embodiment 37. The cell engager of embodiment 36, wherein said polypeptide expressed on the surface of T cells is a CD3 polypeptide.
   Embodiment 38. The cell engager of embodiment 36, wherein said second antigen binding domain is an antigen binding domain set forth in FIG. 18.
   Embodiment 39. The cell engager of any one of embodiments 32-35, wherein said second antigen binding domain binds to a polypeptide expressed on the surface of NK cells.
   Embodiment 40. The cell engager of embodiment 39, wherein said polypeptide expressed on the surface of NK cells is a CD16a, NKG2A, NKG2D, NKp30, NKp44, or NKp46 polypeptide.
   Embodiment 41. The cell engager of embodiment 39, wherein said second antigen binding domain is an antigen binding domain set forth in FIG. 19.
   Embodiment 42. The cell engager of any one of embodiments 32-41, wherein said cell engager comprises a third antigen binding domain.
   Embodiment 43. The cell engager of embodiment 42, wherein said third antigen binding domain binds to a polypeptide expressed on the surface of NK cells.
   Embodiment 44. The cell engager of embodiment 43, wherein said polypeptide expressed on the surface of NK cells is a CD16a, NKG2A, NKG2D, NKp30, NKp44, or NKp46 polypeptide.
   Embodiment 45. The cell engager of embodiment 43, wherein said third antigen binding domain is an antigen binding domain set forth in FIG. 19.
   Embodiment 46. A nucleic acid comprising a nucleic acid sequence encoding at least part of an antibody or an antigen-binding fragment of any one of embodiments 1-24.
   Embodiment 47. The nucleic acid of embodiment 46, wherein said nucleic acid sequence encodes said heavy chain variable domain or region of any one of said (i)-(ii) of embodiment 1.
   Embodiment 48. The nucleic acid of any one of embodiments 46-47, wherein said nucleic acid sequence encodes said light chain variable domain or region of any one of said (i)-(ii) of embodiment 1.
   Embodiment 49. The nucleic acid of any one of embodiments 46-48, wherein said nucleic acid is a viral vector.
   Embodiment 50. The nucleic acid of any one of embodiments 46-48, wherein said nucleic acid is a phagemid.
   Embodiment 51. A nucleic acid comprising a nucleic acid sequence encoding a chimeric antigen receptor of any one of embodiments 25-29 or a cell engager of any one of embodiments 32-45.
   Embodiment 52. The nucleic acid of embodiment 51, wherein said nucleic acid is a viral vector.
   Embodiment 53. The nucleic acid of embodiment 51, wherein said nucleic acid is a phagemid.
   Embodiment 54. A host cell comprising a nucleic acid of any one of embodiments 46-52.
   Embodiment 55. A host cell that expresses a chimeric antigen receptor of any one of embodiments 25-29 or a cell engager of any one of embodiments 32-45.
   Embodiment 56. The host cell of any one of embodiments 54-55, wherein said host cell is a T cell, stem cell, or NK cell.
   Embodiment 57. An antibody-drug conjugate (ADC) comprising an antigen binding domain covalently linked to a drug, wherein said antigen binding domain comprises an antibody or an antigen binding fragment of any one of embodiments 1-24.
   Embodiment 58. The ADC of embodiment 57, wherein said antigen binding domain comprises a scFv having the ability to bind to a CD66e polypeptide.
   Embodiment 59. The ADC of embodiment 57, wherein said antigen binding domain is an IgG having the ability to bind to a CD66e polypeptide.
   Embodiment 60. The ADC of any one of embodiments 57-59, wherein said drug is selected from the group consisting of auristatins, mertansine, or pyrrolobenzodiazepine (PBD) dimers.
   Embodiment 61. A composition comprising an antibody or an antigen binding fragment of any one of embodiments 1-24.
   Embodiment 62. The composition of embodiment 61, wherein said composition comprises said antibody of any one of embodiments 1-10, 21, and 22.
   Embodiment 63. The composition of embodiment 61, wherein said composition comprises said antigen binding fragment of any one of embodiments 11-20, 23, and 24.
   Embodiment 64. A composition comprising a cell engager of any one of embodiments 32-45.
   Embodiment 65. A composition comprising a cell of any one of embodiments 30, 31, and 53-56.
   Embodiment 66. A composition comprising an ADC of any one of embodiments 57-60.
   Embodiment 67. The composition of any one of embodiments 61-66, wherein said composition comprises a checkpoint inhibitor.
   Embodiment 68. The composition of embodiment 67, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.
   Embodiment 69. A method of treating a mammal having cancer, wherein said method comprises administering, to said mammal, a composition of any one of embodiments 61-68.
   Embodiment 70. The method of embodiment 69, wherein said mammal is a human.
   Embodiment 71. The method of any one of embodiments 69-70, wherein said cancer is a CD66e⁺ cancer.
   Embodiment 72. The method of embodiment 71, wherein said CD66e⁺ cancer is selected from the group consisting of CD66e⁺ lung cancer, CD66e⁺ prostate cancer, CD66e⁺ esophageal cancer, CD66e⁺ stomach cancer, CD66e⁺ colorectal cancer, CD66e⁺ liver cancer, CD66e⁺ vaginal cancer, or CD66e⁺ cervical cancer.
   Embodiment 73. The method of any one of embodiments 69-72, wherein the number of cancer cells within said mammal is reduced following said administering step.
   Embodiment 74. A method of treating a mammal having cancer, wherein said method comprises:
(a) administering, to said mammal, said composition of any one of embodiments 98-104, and
(b) administering, to said mammal, a composition comprising a checkpoint inhibitor.
   Embodiment 75. The method of embodiment 74, wherein said mammal is a human.
   Embodiment 76. The method of any one of embodiments 74-75, wherein said cancer is a CD66e⁺ cancer.
   Embodiment 77. The method of embodiment 76, wherein said CD66e⁺ cancer is selected from the group consisting of CD66e⁺ lung cancer, CD66e⁺ prostate cancer, CD66e⁺ esophageal cancer, CD66e⁺ stomach cancer, CD66e⁺ colorectal cancer, CD66e⁺ liver cancer, CD66e⁺ vaginal cancer, or CD66e⁺ cervical cancer.
   Embodiment 78. The method of any one of embodiments 74-77, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.
   Embodiment 79. The method of any one of embodiments 74-78, wherein the number of cancer cells within said mammal is reduced following said administering steps (a) and (b).
   Embodiment 80. A method for binding a binding molecule to a CD66e polypeptide, wherein said method comprises contacting said CD66e polypeptide with an antibody or an antigen binding fragment of any one of embodiments 1-24.
   Embodiment 81. The method of embodiment 80, wherein said contacting is performed in vitro.
   Embodiment 82. The method of embodiment 80, wherein said contacting is performed in vivo.
   Embodiment 83. The method of embodiment 82, wherein said contacting is performed within a mammal by administering said antibody or said antigen binding fragment to said mammal.
   Embodiment 84. The method of embodiment 84, wherein said mammal is a human.
   Embodiment 85. A method for binding a binding molecule to a CD66e polypeptide, wherein said method comprises contacting said CD66e polypeptide with a chimeric antigen receptor of any one of embodiments 25-29, a cell engager of any one of embodiments 32-45, or an ADC of any one of embodiments 57-60.
   Embodiment 86. The method of embodiment 85, wherein said contacting is performed in vitro.
   Embodiment 87. The method of embodiment 85, wherein said contacting is performed in vivo.
   Embodiment 88. The method of embodiment 87, wherein said contacting is performed within a mammal by administering said chimeric antigen receptor, said cell engager, or said ADC to said mammal.
   Embodiment 89. The method of embodiment 88, wherein said mammal is a human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

2. The antibody of claim 1, wherein said antibody comprises the ability to bind to SEQ ID NO:150 or SEQ ID NO:151.

3. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); or
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO: 19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions).

4. The antigen binding fragment of claim 3, wherein said antigen binding fragment comprises the ability to bind to SEQ ID NO:150 or SEQ ID NO:151.

5. A chimeric antigen receptor comprising an antigen binding domain, a hinge, a transmembrane domain, and one or more signaling domains, wherein said antigen binding domain comprises an antibody or an antigen-binding fragment of any one of claims 1-4.

6. The chimeric antigen receptor of claim 5, wherein said antigen binding domain comprises a scFv having the ability to bind to a CD66e polypeptide.

7. The chimeric antigen receptor of any one of claims 5-6, wherein said hinge comprises a hinge set forth in FIG. 13.

8. The chimeric antigen receptor of any one of claims 5-7, wherein said transmembrane domain comprises a transmembrane domain set forth in FIG. 14.

9. The chimeric antigen receptor of any one of claims 5-8, wherein said chimeric antigen receptor comprises one or more signaling domains set forth in FIG. 15.

10. A cell comprising a chimeric antigen receptor of any one of claims 5-9.

11. The cell of claim 10, wherein said cell is a T cell, a stem cell, or an NK cell.

12. A cell engager comprising a first antigen binding domain, a linker, and a second antigen binding domain, wherein said first antigen binding domain comprises an antibody or an antigen-binding fragment of any one of claims 1-4.

13. The cell engager of claim 12, wherein said first antigen binding domain comprises a scFv having the ability to bind to a CD66e polypeptide.

14. The cell engager of claim 12, wherein said first antigen binding domain is an IgG having the ability to bind to a CD66e polypeptide.

15. The cell engager of any one of claims 12-14, wherein said linker comprises a linker set forth in FIG. 10 or FIG. 13.

16. The cell engager of any one of claims 12-15, wherein said second antigen binding domain binds to a polypeptide expressed on the surface of T cells.

17. The cell engager of claim 16, wherein said polypeptide expressed on the surface of T cells is a CD3 polypeptide.

18. The cell engager of claim 16, wherein said second antigen binding domain is an antigen binding domain set forth in FIG. 18.

19. The cell engager of any one of claims 12-15, wherein said second antigen binding domain binds to a polypeptide expressed on the surface of NK cells.

20. The cell engager of claim 19, wherein said polypeptide expressed on the surface of NK cells is a CD16a, NKG2A, NKG2D, NKp30, NKp44, or NKp46 polypeptide.

21. The cell engager of claim 19, wherein said second antigen binding domain is an antigen binding domain set forth in FIG. 19.

22. The cell engager of any one of claims 12-21, wherein said cell engager comprises a third antigen binding domain.

23. The cell engager of claim 22, wherein said third antigen binding domain binds to a polypeptide expressed on the surface of NK cells.

24. The cell engager of claim 23, wherein said polypeptide expressed on the surface of NK cells is a CD16a, NKG2A, NKG2D, NKp30, NKp44, or NKp46 polypeptide.

25. The cell engager of claim 23, wherein said third antigen binding domain is an antigen binding domain set forth in FIG. 19.

26. A nucleic acid comprising a nucleic acid sequence encoding at least part of an antibody or an antigen-binding fragment of any one of claims 1-4.

27. A nucleic acid comprising a nucleic acid sequence encoding a chimeric antigen receptor of any one of claims 5-9 or a cell engager of any one of claims 12-25.

28. A host cell comprising a nucleic acid of any one of claims 26-27.

29. A host cell that expresses a chimeric antigen receptor of any one of claims 5-9 or a cell engager of any one of claims 12-25.

30. The host cell of any one of claims 28-29, wherein said host cell is a T cell, stem cell, or NK cell.

31. An antibody-drug conjugate (ADC) comprising an antigen binding domain covalently linked to a drug, wherein said antigen binding domain comprises an antibody or an antigen binding fragment of any one of claims 1-4.

32. The ADC of claim 31, wherein said antigen binding domain comprises a scFv having the ability to bind to a CD66e polypeptide.

33. The ADC of claim 31, wherein said antigen binding domain is an IgG having the ability to bind to a CD66e polypeptide.

34. The ADC of any one of claims 31-33, wherein said drug is selected from the group consisting of auristatins, mertansine, or pyrrolobenzodiazepine (PBD) dimers.

35. A composition comprising an antibody or an antigen binding fragment of any one of claims 1-4.

36. A composition comprising a cell engager of any one of claims 12-25.

37. A composition comprising a cell of any one of claims 28-30.

38. A composition comprising an ADC of any one of claims 31-34.

39. The composition of any one of claims 35-38, wherein said composition comprises a checkpoint inhibitor.

40. The composition of claim 39, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

41. A method of treating a mammal having cancer, wherein said method comprises administering, to said mammal, a composition of any one of claims 35-40.

42. The method of claim 41, wherein said mammal is a human.

43. The method of any one of claims 41-42, wherein said cancer is a CD66e⁺ cancer.

44. The method of claim 43, wherein said CD66e⁺ cancer is selected from the group consisting of CD66e⁺ lung cancer, CD66e⁺ prostate cancer, CD66e⁺ esophageal cancer, CD66e⁺ stomach cancer, CD66e⁺ colorectal cancer, CD66e⁺ liver cancer, CD66e⁺ vaginal cancer, or CD66e⁺ cervical cancer.

45. The method of any one of claims 41-44, wherein the number of cancer cells within said mammal is reduced following said administering step.

46. A method of treating a mammal having cancer, wherein said method comprises:
(a) administering, to said mammal, said composition of any one of claims 35-38, and
(b) administering, to said mammal, a composition comprising a checkpoint inhibitor.

47. The method of claim 46, wherein said mammal is a human.

48. The method of any one of claims 46-47, wherein the number of cancer cells within said mammal is reduced following said administering steps (a) and (b).

49. A method for binding a binding molecule to a CD66e polypeptide, wherein said method comprises contacting said CD66e polypeptide with an antibody or an antigen binding fragment of any one of claims 1-4.

50. A method for binding a binding molecule to a CD66e polypeptide, wherein said method comprises contacting said CD66e polypeptide with a chimeric antigen receptor of any one of claims 5-9, a cell engager of any one of claims 12-25, or an ADC of any one of claims 31-34.
